(19) United States
(12) Patent Application Publication
(10) Pub. No.: US 2024/0343729 A1
(43) Pub. Date: Oct. 17, 2024

(54) IMIDAZOLE ORGANIC COMPOUNDS AND THEIR USE AGAINST INFLAMMATORY BOWEL DISEASE

(71) Applicant: BenevolentAI Cambridge Limited, London (GB)

(72) Inventors: Joshua ALMOND-THYNNE, London (GB); Lee PATIENT, London (GB); Edward I. BALMOND, London (GB); Nicola ROBAS, London (GB); Monica Polidoro BELICH, London (GB)

(21) Appl. No.: 18/293,645

(22) PCT Filed: Jul. 29, 2022
(86) PCT No.: PCT/GB2022/052010

§ 371 Date: Jan. 30, 2024

(30) Foreign Application Priority Data
Jul. 30, 2021 (GB) 2111001.0

(51) Int. Cl. C07D 487/04; A61K 31/52; A61K 31/5377; A61K 31/541; A61P 1/00; A61P 1/04; C07D 471/04; C07D 473/34; C07D 519/00

(52) U.S. Cl. CPC C07D 487/04; A61K 31/52; A61K 31/5377; A61K 31/541; A61P 1/00; A61P 1/04; C07D 471/04; C07D 473/34; C07D 519/00

(57) ABSTRACT

The present invention relates to compounds of Formula (I) or a pharmaceutically acceptable salt, solvate, hydrate, tautomer, optical isomer, and/or N-oxide thereof. The invention also relates to the processes for the preparation of those compounds, pharmaceutical compositions comprising those compounds, and the uses of those compounds in treating diseases or conditions associated with inflammatory bowel disease (IBD), in particular ulcerative colitis (UC) and Crohn's disease (CD).

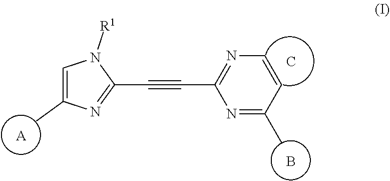

### TECHNICAL FIELD OF THE INVENTION

The present invention relates to compounds of Formula (I) or a pharmaceutically acceptable salt, solvate, hydrate, tautomer, optical isomer, N-oxide, and/or prodrug thereof. The invention also relates to the processes for the preparation of those compounds, pharmaceutical compositions comprising those compounds, and the uses of those compounds in treating diseases or conditions associated with inflammatory bowel disease (IBD), in particular ulcerative colitis (UC) and Crohn's disease (CD).

### BACKGROUND TO THE INVENTION

Inflammatory bowel diseases are characterised by chronic uncontrolled inflammation affecting the gastro-intestinal tract and leading to multiple symptoms such as weight loss, abdominal pain, recurrent diarrhoea and bleeding. The prevalence of IBD is around 1 in 1000 people in Europe, with higher prevalence and incidence rates observed in westernized and industrialized countries (Loftus E V, Clinical epidemiology of inflammatory bowel disease: Incidence, prevalence, and environmental influences, Gastroenterology. 126(6):1504-17200 (2004)). Peak incidence occurs in the second to fourth decade of life.

Ulcerative colitis (UC) and Crohn's disease (CD) are chronic, immune-mediated disorders that are collectively referred to as inflammatory bowel diseases (IBD). Both CD and UC are characterised by dysregulated, aberrant immune responses of the intestinal mucosa. The goals of treatment for both CD and UC are to achieve symptom control, clinical remission, and to prevent disease progression by eliminating or controlling the inflammatory burden (Rubin, D. T., Ananthakrishnan, et al., Clinical Guideline: Ulcerative Colitis in Adults. Am. J. Gastroenterol. 114, 384-413 (2019)).

UC and CD share many pathologic mechanisms. Antigen-presenting cells, Th1, Th2, T regulatory cells and Th17 T-cells are activated in both UC and CD which results in upregulated expression of multiple proinflammatory cytokines and chemokines (Sartor, R. B. Mechanisms of Disease: pathogenesis of Crohn's disease and ulcerative colitis. Nat Clin Pract Gastr. 3, 390-407 (2006)). There are many common pathways and cytokines that are upregulated in both diseases that play important roles in disease pathology (Ramos, G. P. & Papadakis, K. A. Mechanisms of Disease: Inflammatory Bowel Diseases. Mayo Clin Proc 94, 155-165 (2019)), including the cytokines that the applicant has measured in patient ex-vivo colon biopsies—IL-6 (Mudter, J. & Neurath, M. F. II-6 signaling in inflammatory bowel disease: Pathophysiological role and clinical relevance. Inflamm Bowel Dis 13, 1016-1023 (2007)), IL-8 (Daig, R. et al. Increased interleukin 8 expression in the colon mucosa of patients with inflammatory bowel disease. Gut 38, 216 (1996)), and TNF-α (Friedrich, M., Pohin, M. & Powrie, F. Cytokine Networks in the Pathophysiology of Inflammatory Bowel Disease. Immunity 50, 992-1006 (2019)). Given these shared disease mechanisms between both UC and CD, it is well understood, and supported by a strong rationale, that a therapy effective in UC would be effective in CD, e.g. it has been demonstrated clinically that blockade of TNF alpha by neutralizing monoclonal antibodies treats both active CD and UC (Jsrnerot, G. et al. Infliximab as Rescue Therapy in Severe to Moderately Severe Ulcerative Colitis: A Randomized, Placebo-Controlled Study. Gastroenterology 128, 1805-1811 (2005); Targan, S. R. et al. A Short-Term Study of Chimeric Monoclonal Antibody cA2 to Tumor Necrosis Factor α for Crohn's Disease. New Engl J Medicine 337, 1029-1036 (1997)).

Genetic studies have also shown that UC and CD share a large number of genes involved in disease pathology, with only a small number of genes specific to each disease (Waterman, M. et al. Distinct and overlapping genetic loci in crohn's disease and ulcerative colitis: Correlations with pathogenesis. Inflamm Bowel Dis 17, 1936-1942 (2011)). A combined genome-wide analysis of CD and UC showed that 110 out of the 163 loci that meet genome-wide significance thresholds are associated with both diseases with 50 of these having indistinguishable effect size in CD and UC, and a large proportion of the remainder showing similar directionality in the 2 diseases (Jostins, L. et al. Host-microbe interactions have shaped the genetic architecture of inflammatory bowel disease. Nature 491, 119-124 (2012)). This degree of sharing of genetic risk suggests that nearly all of the biological mechanisms involved in one disease have some role in the other and therefore that an efficacious treatment for UC would have therapeutic benefit for CD.

There is currently no cure for UC or CD. Therapeutic strategies include interventions on lifestyle habits and medical and surgical treatments. Pharmacological management includes corticosteroids, immunosuppressant agents and anti-tumor necrosis factor (TNF)-α biologics (Baumgart et al., Inflammatory bowel disease: clinical aspects and established and evolving therapies, Lancet. 369(9573), 1641-57, (2007)).

There therefore remains an urgent need for treatments for inflammatory bowel disease, and in particular ulcerative colitis and Crohn's disease.

It is an object of the present invention to provide for treatments which could be used in a range of inflammatory bowel disease conditions. It would be advantageous, if such treatments could be used to treat inflammatory bowel diseases such as ulcerative colitis and Crohn's disease.

### SUMMARY OF THE INVENTION

In a first aspect of the invention there is provided a compound of Formula (I)

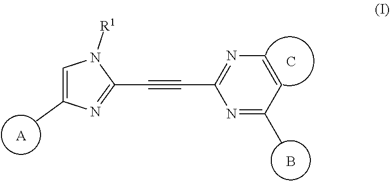

or a pharmaceutically acceptable salt, solvate, hydrate, tautomer, optical isomer, N-oxide, and/or prodrug thereof, wherein
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
Ring C is a 5- or 6-membered carbocycle, 5- or 6-membered heterocycle or 5- or 6-membered heteroaryl group, optionally substituted with one or more R²
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle;
each R² is independently selected from the group consisting of halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
R³ is a selected from H and (C₁-C₄)alkyl, wherein the (C₁-C₄)alkyl is optionally substituted with one or more substituents selected from halo, —OH, and —NR¹⁵R¹⁶; and
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ may be taken together with the atom to which they are attached to form a 5- or 6-membered heterocycle that is optionally substituted with one or more substituents selected from halo and (C₁-C₄)alkyl.

Compounds of Formula (I), and any sub-formula thereof, are “compounds of the invention”, or “the compounds”.

The second aspect of the invention provides pharmaceutical compositions comprising a compound of the invention.

The compounds of the invention may inhibit PDE10A at a level suitable to prevent or treat IBD, and in particular ulcerative colitis and/or Crohn's disease, for the reasons set out below.

It has been surprisingly and advantageously found that the selective inhibition of PDE10A with a small molecule inhibitor reduces inflammatory cytokine levels in colon samples from IBD patients and therefore this represents an unexpected and promising treatment for inflammatory bowel diseases, and in particular, ulcerative colitis and Crohn's disease.

Cyclic nucleotide phosphodiesterases (PDEs) are a family of enzymes that catalyse the degradation of the cyclic nucleotide second messengers cyclic adenosine monophosphate (cAMP) and cyclic guanosine monophosphate (cGMP). Intracellular levels of the cAMP and cGMP are regulated by both their rates of synthesis (by adenylate cyclases and guanylate cyclases respectively) and their hydrolysis by phosphodiesterases. By regulating the duration and amplitude of the cAMP and cGMP second messenger signals, PDEs play critical regulatory roles in signal transduction.

There are 11 different PDE subtypes (PDE1 to PDE11), each encoding PDEs with unique substrate specificities, kinetics, allosteric regulators, tissue-expression profiles, and pharmacological sensitivities. PDE10A is able to hydrolyse both cAMP and cGMP. PDE10A hydrolyzes cAMP with a Kₘ of 0.05 μM and cGMP with a Kₘ of 3 μM. Although PDE10A has a lower Kₘ for cAMP, the Vₘₐₓ ratio cGMP/cAMP is 4.7 indicating a higher specific activity for cGMP. Taken together this suggests that PDE10A is a cAMP-inhibited cGMP phosphodiesterase.

In normal tissue, PDE10A has a restricted expression pattern. High PDE10A RNA levels are detected only in the striatum (caudate nucleus and putamen) of the brain, and the testes (Fujishige K, Kotera J, Michibata H, Yuasa K, Takebayashi S, Okumura K, Omori K. Cloning and characterization of a novel human phosphodiesterase that hydrolyzes both cAMP and cGMP (PDE10A). J Biol Chem. 274, 18438-18445 (1999)). To date inhibitors of PDE10A have mainly been investigated for neurological conditions including schizophrenia and Parkinson's disease (Geerts H, Spiros A, Roberts P. Phosphodiesterase 10 inhibitors in clinical development for CNS disorders. Expert Rev Neurother. 17(6), 553-560 (2017)). PDE10A has not been investigated extensively for inflammation. A search of the literature identified one paper (Garcia A M et al. Targeting PDE10A GAF Domain with Small Molecules: A Way for Allosteric Modulation with AntiInflammatory Effects. Molecules., 1472, 22(9), 2017), that described inhibition of LPS-induced nitrite release from the Raw 264.7 macrophage cell line by a PDE10A inhibitor i.e. in a transformed mouse cell line rather than human primary cells. The authors ascribed the effect seen to the cAMP hydrolytic activity of PDE10A rather than its cGMP activity.

The present inventors have surprisingly found that PDE10A inhibitors described herein can reduce the levels of inflammatory cytokines, that are a hallmark of IBD, in colon biopsies taken from IBD patients, and therefore represent a new therapeutic opportunity for the treatment of these diseases.

The inflammatory bowel diseases may comprise ulcerative colitis and/or Crohn's disease. It is well understood that any treatment for ulcerative colitis is likely to be suitable to treat Crohn's disease, and vice-versa. This is demonstrated for the present compounds in the Examples below.

The present invention provides compounds, that may be PDE10A inhibitors, for use in the prevention and/or treatment of inflammatory bowel disease. Suitably the inflammatory bowel disease is selected from ulcerative colitis and/or Crohn's disease. This is the third aspect of the invention.

### DESCRIPTION OF FIGURES

FIG. 1 contains plots showing RNA expression of PDE10A in normal tissue. The plots represent baseline gene expression of PDE10A and GUCY2C (guanylate cyclase 2C) in healthy samples based on GTEx data, where the X-axis represents tissue, y-axis represents log₂ transformed expression.

FIG. 2 contains volcano plots showing differential RNA expression of PDE10A and GUCY2C. The volcano plots show differential gene expression for a selected comparison, where the x-axis represents log fold change (FC) and y-axis represents log₁₀ transformed adjusted p-value (FDR). Horizontal dotted line is FDR=0.05 threshold and values above the dotted line are considered significant. Values to the right of the central axis indicate upregulation, values to the left of the central axis indicate down regulation. The OmicSoft differential expression datasets used in the analysis were as follows: colonic mucosa—OmicSoft Project names: GSE14580, GSE16879, GSE36807, GSE59071, GSE65114, GSE73661; colon—OmicSoft Project names: GSE10191, GSE10616, GSE6731, GSE9686.

FIG. 3 is a graph showing the effect of a PDE10A inhibitor, PF-02545920, on isolated human neutrophil activation in response to IL-8.

FIG. 4 contains graphs showing PF-02545920 and TAK-063 inhibiting the release of inflammatory cytokines IL-6 and IL-8 in ex-vivo cultures of colon biopsy samples from a UC patient (US donor 1). (A) Effect of PDE10A inhibitors on IL-6 levels, (B) Effect of PDE10A inhibitors on IL-8 levels, (n=2; Mean±SD), wherein Pred=prednisolone, Tofa=tofacitinib.

FIG. 5 contains graphs showing PF-02545920 and TAK-063 inhibiting the release of inflammatory cytokines IL-6 and IL-8 in ex-vivo cultures of colon biopsy samples from a UC patient (UC donor 2). (A) Effect of PDE10A inhibitors on IL-6 levels, (B) Effect of PDE10A inhibitors on IL-8 levels, (n=2; Mean±SD), wherein Pred=prednisolone, Tofa=tofacitinib.

FIGS. 6 to 9 contain graphs showing the effect of the compound of Reference Example A (FIG. 6A), Reference Example B (FIG. 6B), Reference Example C (FIG. 7A), Reference Example D (FIG. 7B), Reference Example E (FIG. 8A), Reference Example F (FIG. 8B), and Reference Example G (FIG. 9) on inflammatory cytokine release from ex-vivo ulcerative colitis colon tissue (UC donor 3).

FIG. 10 contains graphs showing PF-02545920 (1 μM) inhibiting release of the inflammatory cytokine TNFα in ex-vivo cultures of inflamed colon tissue obtained from surgical resection from treatment-refractory UC patients. (A) UC donor 4, (B) UC donor 5 (n=5; Mean±SD; *p<0.05).

FIG. 11 shows that PF-2545920 inhibits the spontaneous release of inflammatory cytokines IL-6 and IL-8 in ex-vivo cultures of inflamed CD colon tissue. Graph (A) CD donor 1, graph (B) CD donor 2 (n=2; Mean±SD).

### DETAILED DESCRIPTION OF THE INVENTION

The applicant has found that certain compounds may be used to prevent and/or treat diseases or conditions susceptible to PDA10A inhibition. In a first aspect of the invention there is provided a compound of Formula (I)

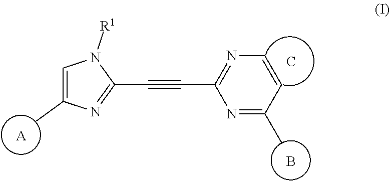

or a pharmaceutically acceptable salt, solvate, hydrate, tautomer, optical isomer, N-oxide, and/or prodrug thereof, wherein
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
Ring C is a 5- or 6-membered carbocycle, 5- or 6-membered heterocycle or 5- or 6-membered heteroaryl group, optionally substituted with one or more R²
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle;
each R² is independently selected from the group consisting of halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
R³ is a selected from H and (C₁-C₄)alkyl, wherein the (C₁-C₄)alkyl is optionally substituted with one or more substituents selected from halo, —OH, and —NR¹⁵R¹⁶; and
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl.

As used herein, “optionally substituted” means the group referred to can be unsubstituted, or substituted at one or more positions, i.e. one, two, three, four, five, six or more positions, by any one or any combination of the substituents, such as those listed thereafter.

The term “halo” means a fluoro (F), chloro (Cl), bromo (Br), or iodo (I) group. Unless otherwise stated, it is preferrable that the halo is fluoro at each instance.

The term “(C₁-C₄)alkyl” refers to a fully saturated branched, unbranched or cyclic hydrocarbon moiety having 1, 2, 3, or 4 carbon atoms. Representative examples of (C₁-C₄)alkyl include, but are not limited to, methyl, ethyl, n-propyl, iso-propyl, and cyclopropyl.

As used herein, the term “aryl” means the mono-radical of benzene (CsHs). It will be understood that this may also be referred to as phenyl.

The term “oxo” means ═O. It will be understood that an oxo group is divalent and therefore replaces two hydrogen atoms on a single carbon atom when used as a substituent.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

As used herein, a “5- or 6-membered heteroaryl” is an aromatic ring system that comprises 5 or 6 ring atom, at least one of which is a heteroatom. Unless otherwise stated herein, examples of a 5- or 6-membered heteroaryl include pyrole, pyrazole, imidazole, triazole, furan, thiophene, oxazole, isoxazole. isothiazole, thiazole, pyridine, pyridazine, pyrimidine, pyrazine, and triazine.

The term “4- to 7-membered monocyclic carbocycle” is a saturated single ring system comprising 4 to 7 ring atoms, each of which is a carbon atom. Examples include cyclobutane, cyclopentane, cyclohexane, and cycloheptane. Similarly, the term “5- or 6-membered carbocycle” is a saturated single ring system comprising 5 or 6 ring atoms each of which is a carbon atom. Examples include cyclopentane and cyclohexane.

The term “4- or 6-membered heterocycle” is a saturated single ring system comprising 4 to 6 ring atom, at least one of which is a heteroatom. Examples include azetidine, oxetane, thietane, pyrrolidine, tetrahydrofuran, tetrahydrothiophene, piperidine, piperazine, tetrahydropyran, dioxane, thiane, dithiane, morpholine, and thiomorpholine. The term “5- or 6-membered heterocycle” is a subset of this containing 5 to 6 ring atom, at least one of which is a heteroatom.

The term “5- or 6-membered heteroaryl” is an aromatic single ring comprising 5 or 6 ring atoms, at least one of which is a heteroatom. Examples include pyrrole, pyrazole, imidazole, triazole, furan, thiophene, oxazole, isoxazole, isothiazole, thiazole, pyridine, pyridazine, pyrimidine, and pyrazine.

The term “6- to 10-membered bicyclic carbocycle or heterocycle” is a saturated ring system comprising 6 to 10 atoms, each of which is a carbon atom in the case of the carbocycle, and at least one of which is a heteroatom in the case of the heterocycle. The ring system contains two rings that are fused (i.e. two atoms are shared common to the two rings), or the ring system is a spiro group in which a single atom is common to the two rings.

Unless specifically mentioned, examples of 6- to 10-membered bicyclic carbocycles that are spiro groups include

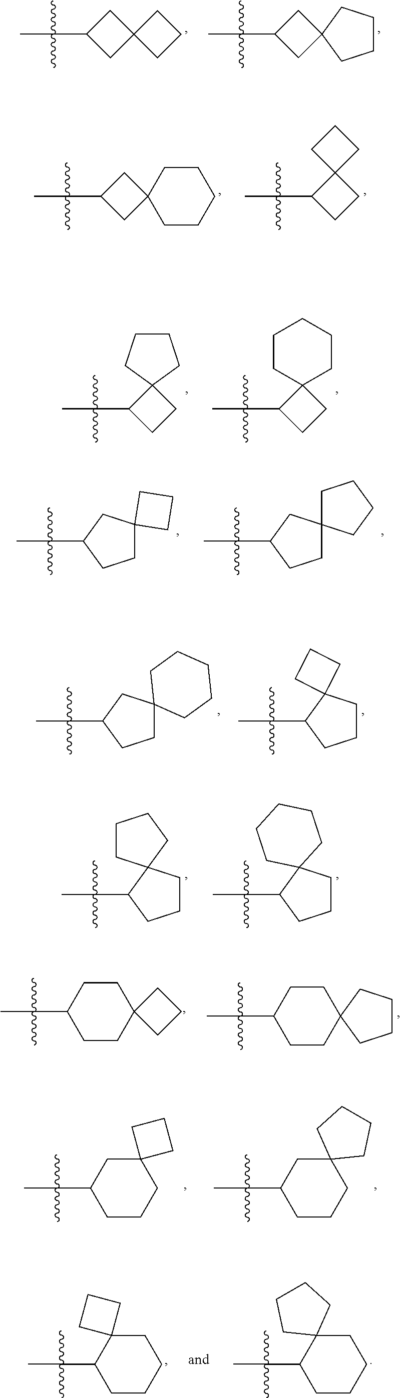

Unless specifically mentioned, examples of 6- to 10-membered bicyclic carbocycles that are fused groups include

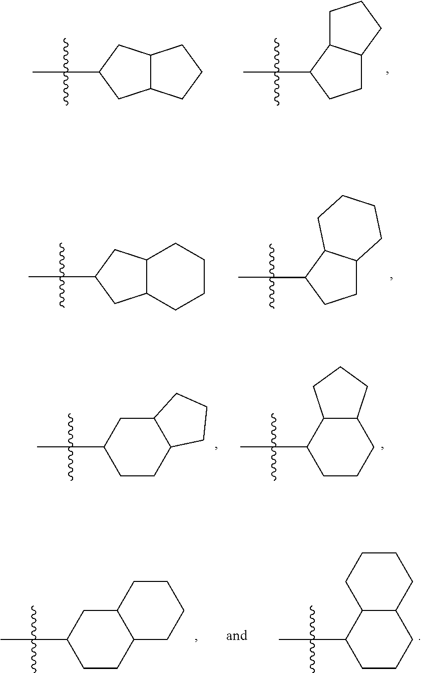

Unless specifically mentioned, examples of 6- to 10-membered bicyclic heterocycles that are spiro groups include

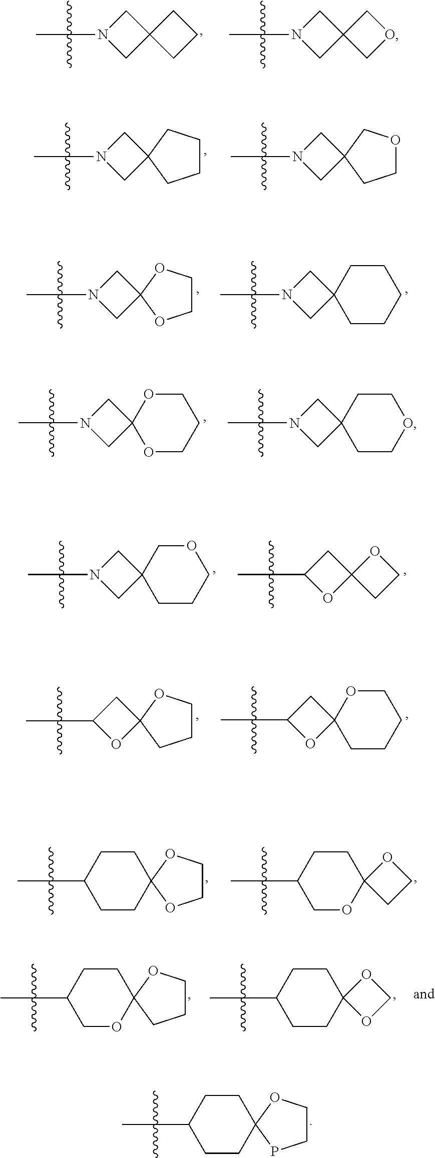

Unless specifically mentioned, examples of 6- to 10-membered bicyclic heterocycles that are fused groups include

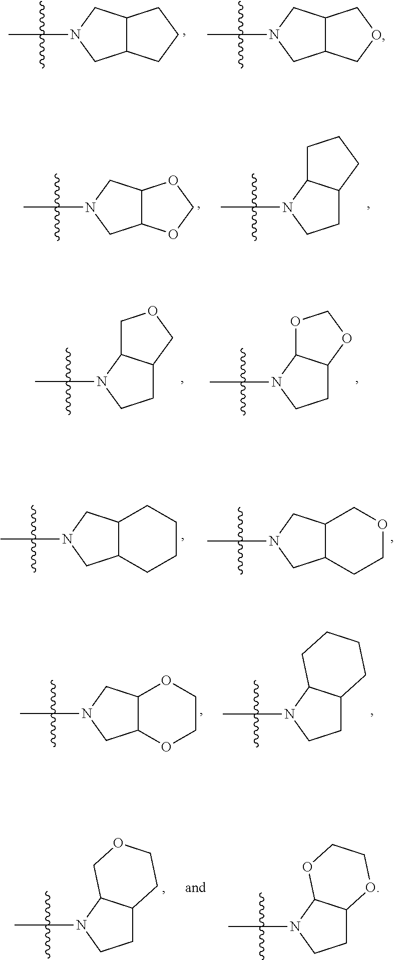

### Ring C

As mentioned above, Ring C may be a 5- or 6-membered carbocycle, 5- or 6-membered heterocycle or 5- or 6-membered heteroaryl group. Each of those groups may be optionally substituted with one or more R². This means that the Ring C and the adjoining pyrimidine may form a fused fully-aromatic bicyclic system, or form a fused bicyclic system in which one ring (the pyrimidine) is aromatic and the other is saturated (albeit except for the two common bridging carbons that are unsaturated).

Options for the pyrimidine/Ring C bicycle of Formula (I) include pyrimidine fused to a 5- or 6-membered heteroaryl group selected from optionally substituted pyrole, pyrazole, imidazole, triazole, furan, thiophene, oxazole, isoxazole. isothiazole, thiazole, pyridine, pyridazine, pyrimidine, pyrazine, and triazine.

Options for the pyrimidine/Ring C bicycle of Formula (I) include pyrimidine fused to a 5- or 6-membered heterocycle group selected from optionally substituted pyrrolidine, pyrazolidine, tetrahydrofuran, dioxolane, tetrahydrothiophene, oxathiolane, piperidine, piperazine, tetrahydropyran, dioxane, thiane, dithiane, morpholine, and thiomorpholine, wherein the two bridging carbons in the 5- or 6-membered heterocycle are unsaturated.

Options for the pyrimidine/Ring C bicycle of Formula (I) include pyrimidine fused to a 5- or 6-membered carbocycle group selected from optionally substituted cyclopentane and cyclohexane, wherein the two bridging carbons in the 5- or 6-membered carbocycle are unsaturated.

Non-limiting examples of Ring C and the fused pyrimidine are shown below, and tautomers thereof, each of which may be substituted as set out above.

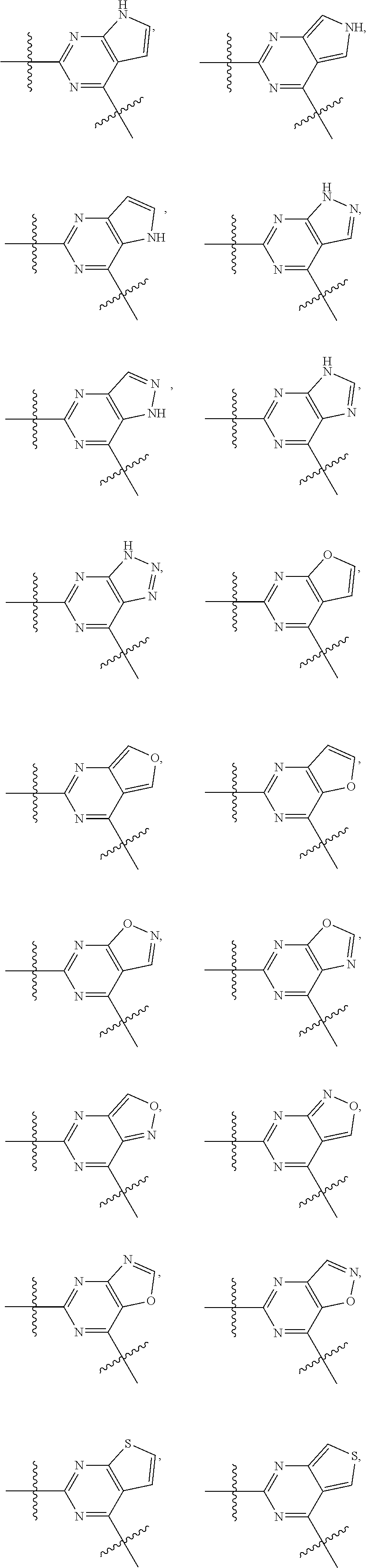

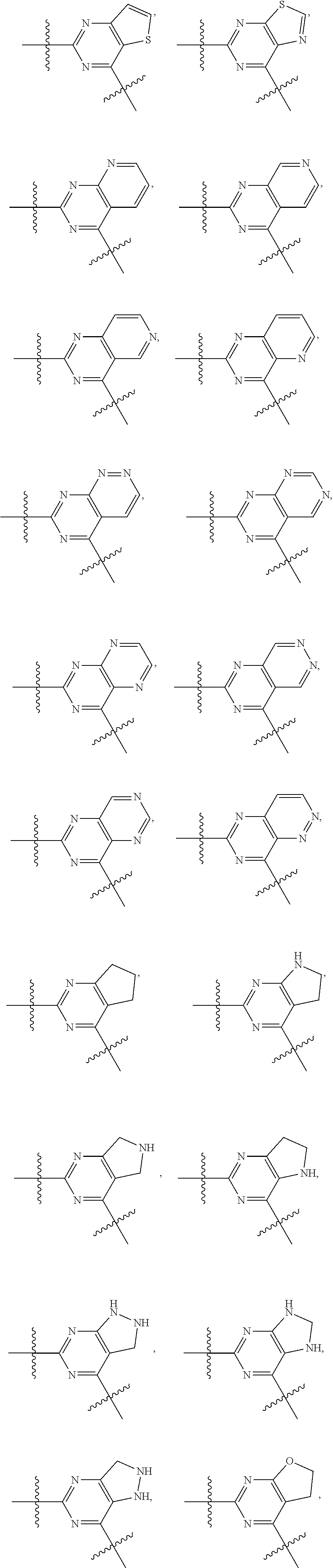

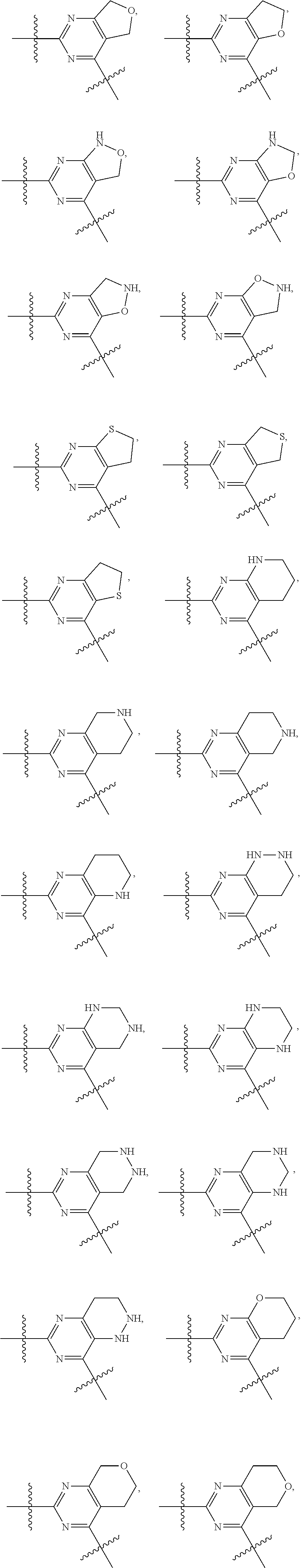

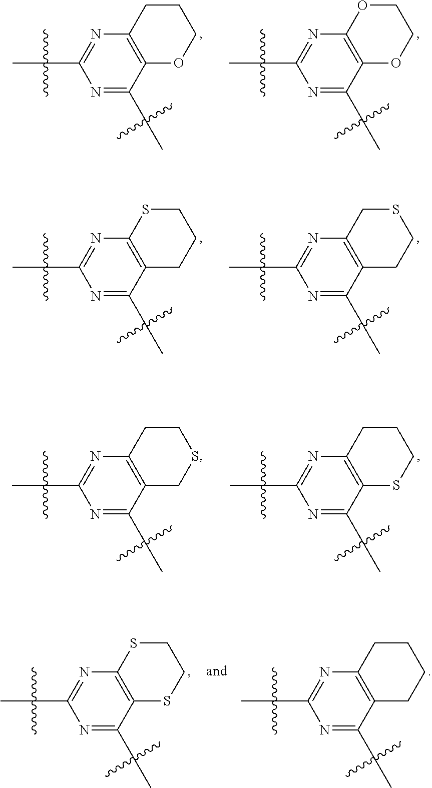

The above list of bicyclic ring systems include regioisomers of the heteroatoms, and tautomers, in Ring C, where applicable and not explicitly mentioned.

It is preferred that Ring C is a 5- or 6-membered heteroaryl or a 5- or 6-membered heterocycle. This is, it is preferrable that Ring C contains at least one heteroatom.

The definition of the groups in the compounds of the invention may differ dependent upon the structure of Ring C as discussed below. If any group below is undefined, then its definition is taken from that mentioned above.

1. Ring C being an Optionally Substituted 5- or 6-Membered Heteroaryl

A particular feature of the first aspect of the invention is that Ring C is a 5- or 6-membered heteroaryl, such as those mentioned above. In this case, it is preferred that Ring C is 5-membered heteroaryl. Particularly preferred a 5-membered heteroaryl Ring C groups are pyrole, pyrazole, imidazole, and triazole, i.e. it is preferred that the heteroatom or heteroatoms in Ring C are nitrogen. Examples of those particularly preferred a 5-membered heteroaryl Ring C groups, in combination with the fused pyrimidine, include but are not limited to

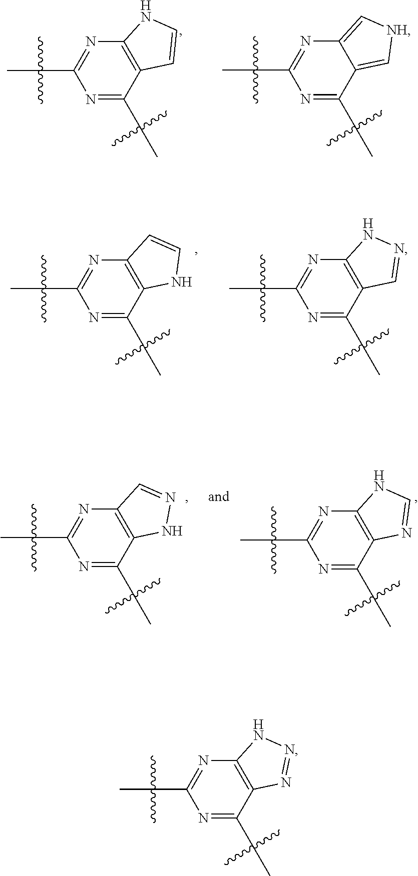

each of which may be optionally substituted with one or more R². This includes substitution of the H of the NH group at each instance.

It is preferred that the 5-membered heteroaryl of Ring C comprises at least two heteroatoms, i.e. at least two nitrogen atoms. This means that Ring C is a pyrazole, imidazole, or triazole.

In view of the above, it is preferable that the compounds of the invention that comprise a 5- or 6-membered heteroaryl as Ring C are represented by a compound of Formula (IA)

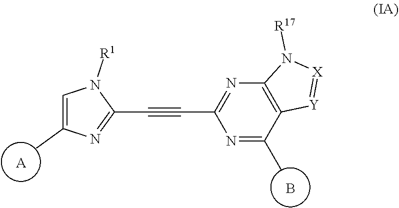

or a pharmaceutically acceptable salt, solvate, hydrate, tautomer, optical isomer, N-oxide, and/or prodrug thereof, wherein
X is N or CR¹⁸;
Y is N or CR¹⁸;
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle;
R³ is a selected from H and (C₁-C₄)alkyl, wherein the (C₁-C₄)alkyl is optionally substituted with one or more substituents selected from halo, —OH, and —NR¹⁵R¹⁶; R¹⁷ is selected from the group consisting of H, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
each R¹⁸ is independently selected from the group consisting of H, halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle; and each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl.

A particular feature of this aspect of the invention is a compound of Formula (IA) in which one of X and Y is N and the other is CR¹⁸. This means that Ring C is a pyrazole or imidazole. Those two groups are particularly preferred for Ring C and are part of the compound of Formula (IA) and compound of Formula (IA″).

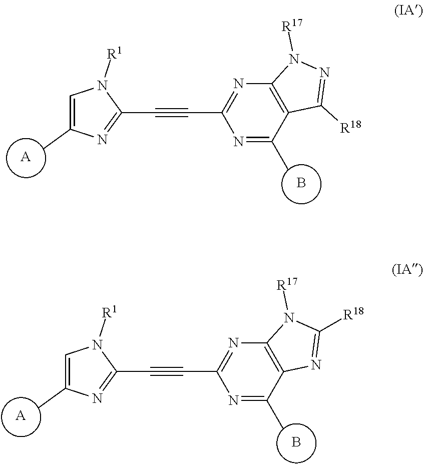

Each R¹⁸ in Formulae (IA), (IA′), and (IA″) may be independently selected from the group consisting of H, halo, and (C₁-C₄)alkyl, wherein the (C₁-C₄)alkyl is optionally substituted with one or more substituents selected from halo. It is preferable that each R¹⁸ is H or halo, and more preferably that each R¹⁸ is H.

As mentioned, when the compound of the invention is of Formula (IA), Ring A may be selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo. It is preferable that Ring A is selected from the group consisting of aryl and 5- or 6-membered heteroaryl, each of which is optionally substituted with one or more substituents selected from halo, (C₁-C₄)alkyl, and —OR³, wherein the (C₁-C₄)alkyl is optionally substituted with one or more halo. In this case, R³ is a selected from H and (C₁-C₄)alkyl, wherein the (C₁-C₄)alkyl is optionally substituted with one or more substituents selected from halo, and —NR¹⁵R¹⁶. Particularly preferred Ring A groups are aryl and pyridyl, optionally substituted with one or more halo and —OR³, wherein R³ is (C₁-C₄)alkyl-NMe₂. The most preferred Ring A groups are

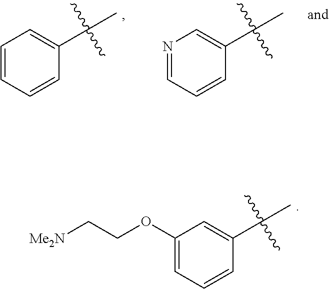 [SMILES: *c1ccccc1 ; *c1cccnc1 ; *c1cccc(OCCN(C)C)c1]

The above definition of Ring A applies equally to Formula (IA′) and (IA″).

When the compound of the invention is a compound of Formula (IA), Ring B may be a monocyclic or bicyclic group.

When it is a bicycle, it may be a 6- to 10-membered bicyclic heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo. It is preferable in this case that Ring B is a preferably a 7- to 10-membered bicyclic heterocycle that is optionally substituted.

Alternatively, when it is a monocycle, Ring B may be a 4- to 7-membered monocyclic heterocycle substituted with at least one of the substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo.

When the compound of the invention is a compound of Formula (IA), the preferred Ring B groups are

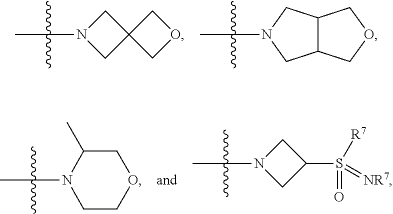

each of which is optionally substituted with one or more halo.

The above definition of Ring B applies equally to Formula (IA′) and (IA″).

When the compound of the invention is a compound of Formula (IA), or Formula (IA′) or (IA″), it is preferable that each R⁷, R¹⁵, and R¹⁶ is independently selected from H and (C₁-C₄)alkyl, wherein the (C₁-C₄)alkyl is optionally substituted with one or more substituents selected from halo.

When the compound of the invention is a compound of Formula (IA), it is preferable that R¹⁷ is selected from the group consisting of H, (C₁-C₄)alkyl, and 4- to 6-membered heterocycle, wherein the (C₁-C₄)alkyl is optionally substituted with one or more substituents selected from halo and 4- to 6-membered heterocycle. This definition of R¹⁷ applies equally to Formula (IA′) and (IA″).

When Ring C is an optionally substituted 5- or 6-membered heteroaryl, any undefined groups above inherit their definition from the compound of Formula (I).

2. Ring C being an Optionally Substituted 5- or 6-Membered Carbocycle, or an Optionally Substituted 5- or 6-Membered Heterocycle

As an alternative to Ring C being an optionally substituted heteroaryl, it may be an optionally substituted 5- or 6-membered carbocycle, or an optionally substituted 5- or 6-membered heterocycle. Whilst a carbocycle and heterocycle is usually saturated, in the present case, as mentioned above, the two carbon atoms that are common to the pyrimidine and Ring C are unsaturated.

It is preferable that Ring C is a 5- or 6-membered heterocycle. The heterocycle may be optionally substituted with one or more R² as defined for Formula (I). More preferably, Ring C is a 6-membered heterocycle, optionally substituted with one or more R². Out of the envisaged options for Ring C, it is preferred that it is a piperidine (with the two carbon atoms that are common to the pyrimidine and Ring C being unsaturated). A particularly important regioisomer is shown in the compound of Formula (IB).

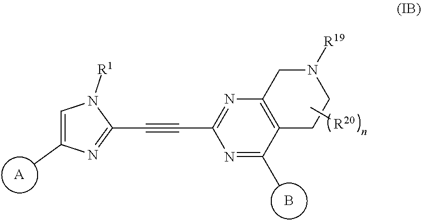

Therefore, a preferred feature is that the compound is of Formula (IB) or a pharmaceutically acceptable salt, solvate, hydrate, tautomer, optical isomer, N-oxide, and/or prodrug thereof, wherein
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle
R³ is a selected from H and (C₁-C₄)alkyl, wherein the (C₁-C₄)alkyl is optionally substituted with one or more substituents selected from halo, —OH, and —NR¹⁵R¹⁶; and
R¹⁹ is selected from the group consisting of H, (C₁-C₄)alkyl, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4-to 6-membered heterocycle;
each R²⁰ is independently selected from the group consisting of halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl; and
n is 0, 1, 2, 3, 4, 5, or 6.

In the compound of Formula (IB), R¹⁹ may be selected from the group consisting of H, (C₁-C₄)alkyl, and 4- to 6-membered heterocycle, wherein the (C₁-C₄)alkyl is optionally substituted with one or more substituents selected from halo. It is preferred that R¹⁹ is H, (C₁-C₄)alkyl, or

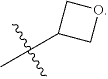 [SMILES: *C1COC1]

In the compound of Formula (IB), each R²⁰ may be independently selected from the group consisting of halo, and (C₁-C₄)alkyl, wherein the (C₁-C₄)alkyl is optionally substituted with one or more substituents selected from halo.

In the compound of Formula (IB), it is preferable that n is 0, 1, 2, 3, or 4. More preferably, n is 0, 1, or 2. Most preferably, n is 0.

When Ring C is a 5- or 6-membered carbocycle or heterocycle (although the heterocycle is preferred), Ring A may be selected from the group consisting of aryl and 6-membered heteroaryl, each of which is optionally substituted with one or more substituents selected from halo. It is preferable that Ring A is aryl or pyridyl. When Ring C is pyridyl, it is preferably 2-pyridyl. However, the most preferred group for Ring A is aryl.

When Ring C is a 5- or 6-membered carbocycle or heterocycle (although the heterocycle is preferred), Ring B may be a 4- to 7-membered monocyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo. It is preferable that Ring B is an optionally substituted 4-to 7-membered monocyclic heterocycle. In particular, Ring B may be a 6-membered heterocycle, optionally substituted with one of more substituents selected from halo, and (C₁-C₄)alkyl, wherein the (C₁-C₄)alkyl is optionally substituted with one or more halo. In this case, Ring B may be a morpholine, preferably with the structure

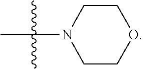 [SMILES: *N1CCOCC1]

The morpholine may be optionally substituted with one or more substituents selected from halo, and (C₁-C₄)alkyl, wherein the (C₁-C₄)alkyl is optionally substituted with one or more halo. Particular examples of Ring B are

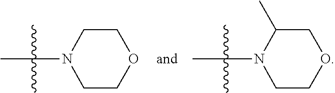

When Ring C is a 5- or 6-membered carbocycle or heterocycle (although the heterocycle is preferred), R¹ may be selected from the group consisting of H and (C₁-C₄)alkyl, wherein the (C₁-C₄)alkyl is optionally substituted with one or more substituents selected from halo. It is preferable that R¹ is H, Me or —CF₃.

When Ring C is an optionally substituted 5- or 6-membered carbocycle, or an optionally substituted 5- or 6-membered heterocycle, such as a compound of Formula (IB), any undefined groups above inherit their definition from the compound of Formula (I).

In view of all of the above, specific compounds of Formula (I) that are particularly useful in the invention are
6-[9-Methyl-2-[2-(1-methyl-4-phenyl-imidazol-2-yl)ethynyl]purin-6-yl]-2-oxa-6-azaspiro[3.3]heptane
6-[9-Methyl-2-[2-[1-methyl-4-(3-pyridyl)imidazol-2-yl]ethynyl]purin-6-yl]-2-oxa-6-azaspiro[3.3]heptane
6-[1-Methyl-6-[2-(1-methyl-4-phenyl-imidazol-2-yl)ethynyl]pyrazolo[3,4-d]pyrimidin-4-yl]-2-oxa-6-azaspiro[3.3]heptane
6-[9-Methyl-2-[2-[4-phenyl-1-(tetrahydropyran-4-ylmethyl)imidazol-2-yl]ethynyl]purin-6-yl]-2-oxa-6-azaspiro[3.3]heptane
6-[9-Methyl-2-[2-[1-(2-morpholinoethyl)-4-phenyl-imidazol-2-yl]ethynyl]purin-6-yl]-2-oxa-6-azaspiro[3.3]heptane
6-[6-[2-(1-Methyl-4-phenyl-imidazol-2-yl)ethynyl]-1-tetrahydropyran-4-yl-pyrazolo[3,4-d]pyrimidin-4-yl]-2-oxa-6-azaspiro[3.3]heptane
6-[9-Methyl-2-[2-(4-phenyl-1H-imidazol-2-yl)ethynyl]purin-6-yl]-2-oxa-6-azaspiro[3.3]heptane
6-[1-(Azetidin-3-ylmethyl)-6-[2-(1-methyl-4-phenyl-imidazol-2-yl)ethynyl]pyrazolo[3,4-d]pyrimidin-4-yl]-2-oxa-6-azaspiro[3.3]heptane
(3aS,6aR)-5-[1-methyl-6-[2-(4-phenyl-1H-imidazol-2-yl)ethynyl]pyrazolo[3,4-d]pyrimidin-4-yl]-1,3,3a,4,6,6a-hexahydrofuro[3,4-c]pyrrole
2-[3-[2-[2-[4-[(3aS,6aR)-1,3,3a,4,6,6α-Hexahydrofuro[3,4-c]pyrrol-5-yl]-1-methyl-pyrazolo[3,4-d]pyrimidin-6-yl]ethynyl]-1H-imidazol-4-yl]phenoxy]-N,N-dimethyl-ethanamine
4-[2-[2-(1-Methyl-4-phenyl-imidazol-2-yl)ethynyl]-5,6,7,8-tetrahydropyrido[3,4-d]pyrimidin-4-yl]morpholine
(3R)-3-Methyl-4-[2-[2-(1-methyl-4-phenyl-imidazol-2-yl)ethynyl]-5,6,7,8-tetrahydropyrido[3,4-d]pyrimidin-4-yl]morpholine
(3S)-3-Methyl-4-[2-[2-(1-methyl-4-phenyl-imidazol-2-yl)ethynyl]-5,6,7,8-tetrahydropyrido[3,4-d]pyrimidin-4-yl]morpholine
4-[2-[2-(1-Methyl-4-phenyl-imidazol-2-yl)ethynyl]-7-(oxetan-3-yl)-6,8-dihydro-5H-pyrido[3,4-d]pyrimidin-4-yl]morpholine
4-[7-(Cyclopropylmethyl)-2-[2-(1-methyl-4-phenyl-imidazol-2-yl)ethynyl]-6,8-dihydro-5H-pyrido[3,4-d]pyrimidin-4-yl]morpholine
(3aR,6aS)-5-(1-(Cyclopropylmethyl)-6-((1-methyl-4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(1-Ethyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(1-isoPropyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(6-((1-Methyl-4-phenyl-1H-imidazol-2-yl)ethynyl)-1-(oxetan-3-yl)-1H-pyrazolo[3,4-d]pyrimidin-4-yl)hexahydro-1H-furo[3,4-c]pyrrole
(3aR,6aS)-5-(9-Methyl-2-((1-methyl-4-phenyl-1H-imidazol-2-yl)ethynyl)-9H-purin-6-yl)hexahydro-1H-furo[3,4-c]pyrrole
(R)-3-Methyl-4-(9-methyl-2-((1-methyl-4-phenyl-1H-imidazol-2-yl)ethynyl)-9H-purin-6-yl)morpholine
(S)-3-Methyl-4-(9-methyl-2-((1-methyl-4-phenyl-1H-imidazol-2-yl)ethynyl)-9H-purin-6-yl)morpholine
(3aR,6aS)-5-(1-Methyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole
1-Imino-4-(1-methyl-6-((1-methyl-4-phenyl-1H-imidazol-2-yl)ethynyl)-1H-pyrazolo[3,4-d]pyrimidin-4-yl)-116-thiomorpholine-1-oxide
(3aR,6aS)-5-(1-isoPropyl-6-((4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(1-(Cyclopropylmethyl)-6-((4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(1-(Oxetan-3-yl)-6-((4-phenyl-1H-imidazol-2-yl)ethynyl)-1H-pyrazolo[3,4-d]pyrimidin-4-yl)hexahydro-1H-furo[3,4-c]pyrrole
(3aR,6aS)-5-(9-Methyl-2-((4-phenyl-1H-imidazol-2-yl)ethynyl)-9H-purin-6-yl)hexahydro-1H-furo[3,4-c]pyrrole
(R)-3-Methyl-4-(9-methyl-2-((4-phenyl-1H-imidazol-2-yl)ethynyl)-9H-purin-6-yl)morpholine
(S)-3-Methyl-4-(9-methyl-2-((4-phenyl-1H-imidazol-2-yl)ethynyl)-9H-purin-6-yl)morpholine
(3aR,6aS)-5-(9-Methyl-2-((4-(pyridin-3-yl)-1H-imidazol-2-yl)ethynyl)-9H-purin-6-yl)hexahydro-1H-furo[3,4-c]pyrrole
(R)-3-Methyl-4-(9-methyl-2-((4-(pyridin-3-yl)-1H-imidazol-2-yl)ethynyl)-9H-purin-6-yl)morpholine
(S)-3-Methyl-4-(9-methyl-2-((4-(pyridin-3-yl)-1H-imidazol-2-yl)ethynyl)-9H-purin-6-yl)morpholine
   or a pharmaceutically acceptable salt, solvate, hydrate, tautomer, optical isomer, N-oxide, and/or prodrug thereof.

### Additional Definitions

The compounds of the invention may be present as their pharmaceutically acceptable salts. A “pharmaceutically acceptable salt” is intended to mean a salt of a free acid or base of a compound represented by one of the aforementioned Formulae that is non-toxic, biologically tolerable, or otherwise biologically suitable for administration to a subject. Such pharmaceutically acceptable salts are known to those skilled in the art.

Examples of suitable pharmaceutically acceptable salts are those that are pharmacologically effective and suitable for contact with the tissues of subjects without undue toxicity, irritation, or allergic response. Acompound of the invention, may possess a sufficiently acidic group, a sufficiently basic group, or both types of functional groups, and accordingly react with a number of inorganic or organic bases, and inorganic and organic acids, to form a pharmaceutically acceptable salt.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate and trifluoromethylsulfonate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoromethylsulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper, particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Examples of pharmaceutically acceptable salts particularly include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Additionally, any formula given herein is intended to refer also to hydrates and solvates of compounds of the invention, and mixtures thereof, even if such forms are not listed explicitly. A compound of the invention, or pharmaceutically acceptable salt of a compound of the invention, may be obtained as a solvate.

Solvates include those formed from the interaction or complexation of compounds of the invention with one or more solvents, either in solution or as a solid or crystalline form. The solvent may be water, which case the solvates are hydrates. In addition, certain crystalline forms of a compound of the invention, or a pharmaceutically acceptable salt of a compound of the invention, may be obtained as co-crystals. A compound of the invention, or a pharmaceutically acceptable salt of a compound of the invention, may be obtained in a crystalline form.

A compound of the invention, may be obtained in one of several polymorphic forms, as a mixture of crystalline forms, as a polymorphic form, or as an amorphous form. A compound of the invention may convert in solution between one or more crystalline forms and/or polymorphic forms.

Compounds of the invention that contain groups capable of acting as donors and/or acceptors for hydrogen bonds may be capable of forming co-crystals with suitable co-crystal formers. These co-crystals may be prepared from compounds of the invention by known co-crystal forming procedures. Such procedures include grinding, heating, co-subliming, co-melting, or contacting in solution compounds of the invention with the co-crystal former under crystallization conditions and isolating co-crystals thereby formed. Hence the invention further provides co-crystals comprising a compound of the invention.

Any formula given herein is intended to represent compounds having structures depicted by the structural formula as well as certain variations or forms. In particular, compounds of any formula given herein may have asymmetric centres and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof.

Furthermore, certain structures may exist as geometric isomers (i.e., cis and trans isomers), as tautomers, or as atropisomers.

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base addition salts wherein the counter ion is optically active, for example, D-lactate or L-lysine, or racemic, for example, DL-tartrate or DL-arginine.

Where a compound of the invention contains for example, a keto or guanidine group or an aromatic moiety, tautomeric isomerism (‘tautomerism’) can occur. It follows that a single compound may exhibit more than one type of isomerism. Examples of types of potential tautomerisms shown by the compounds of the invention include; amide⇔hydroxyl-imine and keto⇔enol tautomersims.

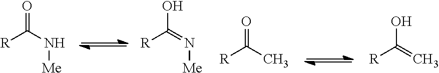

Cis/trans isomers may be separated by conventional techniques well known to those skilled in the art, for example, by chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% ethanol, typically from 2 to 20%. Concentration of the eluate affords the enriched mixture.

Mixtures of stereoisomers may be separated by conventional techniques known to those skilled in the art.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral centre of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

All tautomeric forms are also intended to be included. Tautomers are one of two or more structural isomers that exist in equilibrium and are readily converted from one isomeric form to another. Examples of tautomers include but are not limited to those compounds defined in the claims. Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g. by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-0,0′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions, these less pure preparations of the compounds should contain at least 1%, more suitably at least 5% and preferably from 10 to 59% of a compound of the invention.

When both a basic group and an acid group are present in the same molecule, the compounds of the present invention may also form internal salts, e.g., zwitterionic molecules.

The invention also relates to pharmaceutically acceptable prodrugs of a compound of the invention and treatment methods employing such pharmaceutically acceptable prodrugs.

The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formulae (IA), (IB), (IIA), or (IIB)).

A “pharmaceutically acceptable prodrug” is a prodrug that is non-toxic, biologically tolerable, and otherwise biologically suitable for administration to the subject.

A prodrug is an active or inactive compound that is modified chemically through in vivo physiological action, such as hydrolysis, metabolism and the like, into a compound of the invention following administration of the prodrug to a subject. The compounds of the present invention may themselves be active and/or act as prodrugs which convert in vivo to active compounds. The suitability and techniques involved in making and using pro-drugs are well known by those skilled in the art. Prodrugs can be conceptually divided into two non-exclusive categories, bioprecursor prodrugs and carrier prodrugs. Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity. Carrier prodrugs are drug compounds that contain a transport moiety, e.g., that improve uptake and/or localized delivery to a site(s) of action.

Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde. Moreover, drugs containing an acidic NH group, such as imidazole, imide, indole and the like, have been masked with N-acyloxymethyl groups. Hydroxy groups have been masked as esters and ethers.

The compounds of the invention may also be N-oxides. It will be understood that an N-oxide, or “amine oxide”, is a compound that contains an N—O coordinate covalent bond. Examples of an N-oxide group include the following functional groups.

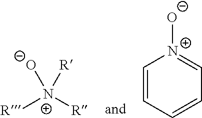

Any formula given herein is also intended to represent unlabelled forms as well as isotopically labelled forms of the compounds. Isotopically labelled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, and fluorine, such as ²H, ³H, ¹¹C, ¹³C, ¹⁴C, ¹³N, ¹⁵N, ¹⁵O, ¹⁷0, ¹⁸O, ¹⁸F, respectively. Such isotopically labelled compounds are useful in metabolic studies (preferably with ¹⁴C), reaction kinetic studies (with, for example ²H or ³H), detection or imaging techniques (such as positron emission tomography (PET) or single-photon emission computed tomography (SPECT)) including drug or substrate tissue distribution assays, or in radioactive treatment of subjects. Substitution with positron emitting isotopes, such as ¹¹C, ¹⁸F, ¹⁵O and ¹³N, can be useful in PET studies for examining substrate receptor occupancy. In particular, an ¹⁸F or ¹¹C labelled compound may be particularly preferred for PET studies. Further, substitution with heavier isotopes such as deuterium (i.e., ²H) may afford certain therapeutic advantages resulting from greater metabolic stability, for example increased in vivo half-life or reduced dosage requirements. Certain isotopically-labelled compounds of the invention for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopically labelled compounds of this invention and prodrugs thereof can generally be prepared by carrying out the procedures disclosed in the schemes or in the examples and preparations described below by substituting a readily available isotopically labelled reagent for a non-isotopically labelled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

If a chemical structure and the associated chemical name do not agree, then the chemical structure takes precedence, unless it is readily understood that the converse is true.

As the compounds of the invention may be used in the prevention and/or treatment of a disease or condition susceptible to PDE10A inhibition, in a third aspect of the invention there is provided a pharmaceutical composition comprising a compound of the invention. As is known, pharmaceutical compositions may comprise one or more excipients in addition to other optional ingredients. It is preferred that the excipients are pharmaceutically acceptable excipients.

The compounds of the invention may be used alone or in combination with one or more additional active ingredients, to formulate pharmaceutical compositions of the invention. A pharmaceutical composition of the invention may comprise (a) an effective amount of at least one compound of the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavouring agents, dyes, and the like and combinations thereof, as would be known to those skilled in the art. Except insofar as any conventional carrier is incompatible with the active ingredient, its use in the therapeutic or pharmaceutical compositions is contemplated.

Pharmaceutical compositions according to the invention may be formulated in conventional manner using readily available ingredients. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

The pharmaceutical compositions can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

When pharmaceutical compositions are tablets or gelatin capsules, they may comprise the active ingredient together (compound of the invention) with
a) diluents, e.g., lactose, polylactone, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

The compounds of the invention may be administered topically. Suitable compositions for topical application to the skin or mucosa (e.g., to the skin and eyes), that is dermally or transdermally, include aqueous solutions, suspensions, ointments, creams, gels, hydrogels, microemulsions, dusting powders, dressings, foams, films, skin patches, wafers, implants, fibres, bandages or sprayable formulations, e.g., for delivery by aerosol or the like. Such topical delivery systems will in particular be appropriate for dermal application, e.g., for the treatment of atopic dermatitis. They are thus particularly suited for use in topical, including cosmetic, formulations well-known in the art. Such may contain solubilizers, stabilizers, tonicity enhancing agents, buffers and preservatives. Typical carriers include alcohol, water, mineral oil, liquid petrolatum, white petrolatum, glycerin, polyethylene glycol and propylene glycol. Penetration enhancers may be incorporated.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant. Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of the invention may be administered either simultaneously with, or before or after, one or more other therapeutic agents. The compound of the invention may be administered separately, by the same or different route of administration, or together in the same pharmaceutical composition as the other agents.

The invention includes a product comprising a compound of the invention and at least one other therapeutic agent as a combined preparation for simultaneous, separate or sequential use in therapy. The therapy may be the treatment of a condition or disorder which is mediated by PDE10A. Products provided as a combined preparation include a composition comprising a compound of the invention and the other therapeutic agent(s) together in the same pharmaceutical composition, or the agent of the invention and the other therapeutic agent(s) in separate form, e.g. in the form of a kit.

### Treatments and Methods

The compounds of the invention may prevent and/or treat inflammatory bowel disease, such as ulcerative colitis and/or Crohn's disease. Without wishing to be bound by theory, the treatment may be achieved due to the ability of the compounds of the invention to inhibit PDE10A.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). The terms “treat”, “treating” or “treatment” also refer to alleviating or ameliorating at least one physical parameter including those which may not be discernible by the patient. The treatment may be a physiological treatment (e.g., stabilization of a discernible symptom), a physical treatment (e.g., stabilization of a physical parameter), or both. The terms “treat”, “treating” or “treatment” also refer to preventing or delaying the onset or development or progression of the disease or disorder.

“Prevention” of a condition or disorder refers to delaying or preventing the onset of a condition or disorder or reducing its severity, as assessed by the appearance or extent of one or more symptoms of said condition or disorder.

The fourth aspect of the invention relates to uses of a compound of the invention, or a pharmaceutical composition comprising a compound of the invention.

A compound of the invention, or a pharmaceutical composition comprising a compound of the invention, may be for use as a medicament.

One feature of the fourth aspect of the invention is therefore the use of a compound of the invention for the manufacture of a medicament. The medicament may be for the prevention and/or treatment (preferably the treatment) of inflammatory bowel disease, such as ulcerative colitis and/or Crohn's disease.

The compound of the invention, or a pharmaceutical composition comprising a compound of the invention, may be for use in the prevention and/or treatment (preferably the treatment) of an inflammatory bowel disease, such as ulcerative colitis and/or Crohn's disease.

There is also described herein a method for the prevention and/or treatment of a disease or condition comprising administering to a subject a compound of the invention, or a pharmaceutical composition comprising a compound of the invention, wherein the disease or condition is susceptible to PDE10A inhibition. The disease or condition susceptible to PDE10A inhibition may be inflammatory bowel disease, such as ulcerative colitis and/or Crohn's disease.

Another method is for the prevention and/or treatment of inflammatory bowel disease comprising administering to a subject a compound of the invention, or a pharmaceutical composition comprising a compound of the invention.

The aforementioned methods are preferably those wherein the inflammatory bowel disease is ulcerative colitis and/or Crohn's disease.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. It is preferable that the subject is a primate, and most preferable that the subject is a human.

Whilst the compound of the invention, and related pharmaceutical compositions, may be used in prevention and/or treatment, it is preferable at every instance that they are for treatment. The methods may therefore be in relation to subject in need of treatment. As used herein, a subject is “in need of” a treatment if such subject would benefit biologically, medically or in quality of life from such treatment.

The compounds of the invention and related pharmaceutical compositions should be provided to subjects in a therapeutically effective amount. The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to at least partially alleviating, inhibiting, preventing and/or ameliorating a condition or disorder which is mediated by PDE10A. In another non-limiting embodiment, the term “a therapeutically effective amount” refers to the amount of the compound of the invention that, when administered to a cell, or a tissue, or a non-cellular biological material, or a medium, is effective to at least partially inhibiting PDE10A activity.

### Preparation of Compounds of the Invention

The compounds of Formula (I) above may be prepared by, or in analogy with, conventional methods. The preparation of intermediates and compounds according to the examples of the present invention may in particular be illuminated by the following Schemes. Definitions of variables in the structures in schemes herein are commensurate with those of corresponding positions in the formulas delineated herein.

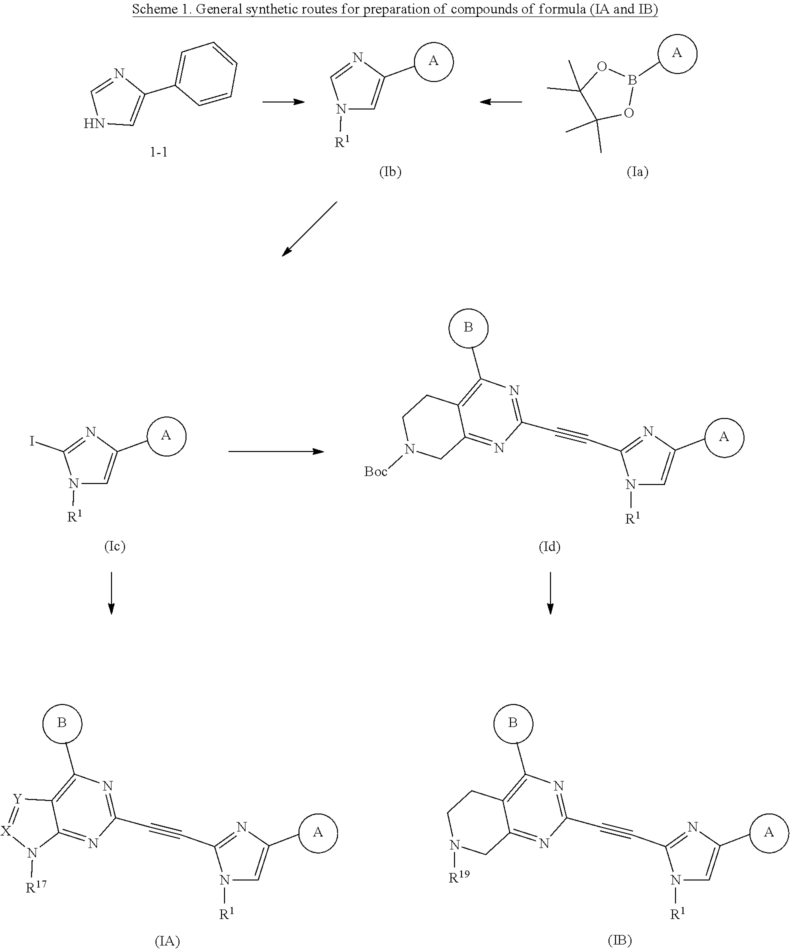

In scheme 1, X and Y are selected from CH or N wherein at least one CR¹⁸ and one N are present. Ring A, Ring B, R¹, R¹⁷ and R¹⁸ are as defined above.

Compounds of general Formulae (IA) and (IB) can easily be prepared by standard means. For example, 4-phenyl-3H-imidazole (1-1) can be converted to compounds of general Formula (Ib) via alkylation with an appropriate alkyl halide. Additionally, compounds of the general Formula (Ia) can be converted to (Ib) via Suzuki-Miyaura coupling with a suitable halogenated imidazole. Compounds of general Formula (Ib) can be reacted with iodine to afford compounds of general Formula (Ic). Compounds of general Formula (Ic) can be reacted with intermediates (Ig) or (Ii) under Sonogashira coupling conditions to afford compounds of general Formula (Id) or (IA) after optional Boc or SEM deprotection. Compounds of general formula (Id) can be converted to compounds of general Formula (IB) via TFA mediated Boc deprotection, and optional alkylation with suitable alkyl halide or reductive amination with suitable ketone.

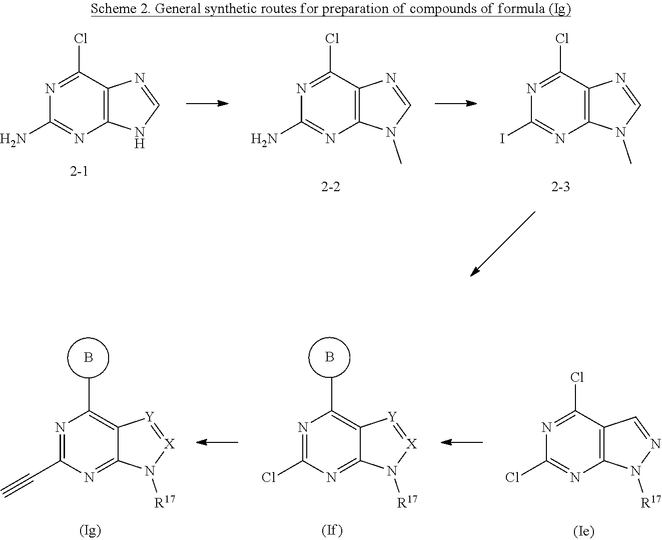

In scheme 2, X and Y are selected from CR¹⁸ or N where at least one CH and one N are present. Ring B, R¹⁷, and R¹⁸ are as defined above.

Compounds of general Formula (Ig) can easily be prepared by standard means. Intermediate (2-1) can be methylated to afford intermediate (2-2), which can subsequently be converted to intermediate (2-3) via a Sandmeyer reaction. Intermediate (2-3) or a compound of general Formula (Ie) can be converted to a compound of general Formula (If) via nucleophilic aromatic substitution and optional alkylation. Compounds of general formula (If) can be converted to compounds of general formula (Ig) via Sonogashira coupling with trimethylsilylacetylene and subsequent deprotection with potassium carbonate.

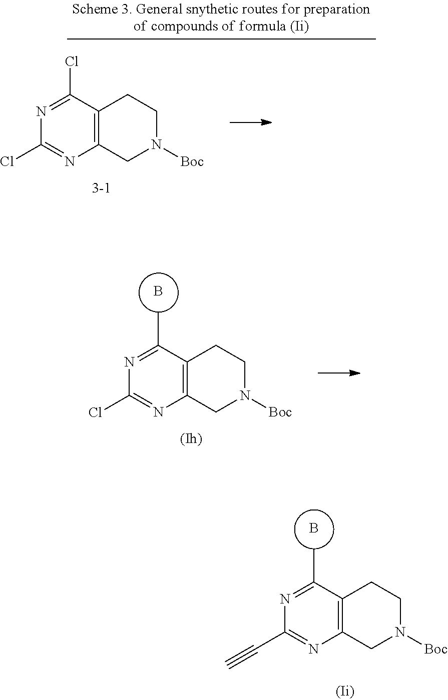

In scheme 2, Ring B is as defined above.

Compounds of general Formula (Ii) can easily be prepared by standard means. Intermediate (3-1) can be converted to compounds of general Formula (Ih) via nucleophilic aromatic. Compounds of general Formula (Ih) can be converted to compounds of general Formula (Ii) via Sonogashira coupling with trimethylsilylacetylene and subsequent deprotection with potassium carbonate.

### EXAMPLES

The compounds of Formula (I), including Formulae (IA) and (IB), above may be prepared by, or in analogy with, conventional methods. The preparation of intermediates according to the examples of the present invention may in particular be illuminated by the following Schemes. Definitions of variables in the structures in schemes herein are commensurate with those of corresponding positions in the formulas delineated herein.

The following abbreviations have been used:
aq Aqueous
Boc tert-Butyloxycarbonyl
DCM Dichloromethane
DIPEA Diisopropylethylamine
DMF Dimethylformamide
dppf 1,1′-Bis(diphenylphosphino)ferrocene
ES+ Electrospray ionization
h hour(s)
HPLC High performance liquid chromatography
LCMS Liquid chromatography-mass spectrometry
min minute(s)
PE Petroleum ether
Rt Retention time
sat Saturated
SEM Trimethylsilylethoxymethyl
TBAF Tetrabutylammonium fluoride
TEA Triethylamine
TFA Trifluoroacetic acid
THF Tetrahydrofuran
TMS Trimethylsilane
UPLC Ultra performance liquid chromatography
XantPhos-Pd-G2 Chloro[(4,5-bis(diphenylphosphino)-9,9-dimethylxanthene)-2-(2′-amino-1,1′-biphenyl)]palladium(II)

### EXAMPLES AND INTERMEDIATE COMPOUNDS

### Experimental Methods

Reactions were conducted at room temperature unless otherwise specified. Preparative chromatography was performed using CombiFlash systems equipped with Isolute Flash II silica columns. Reverse phase column chromatography was performed using CombiFlash systems equipped with RediSep Rf C₁₈ columns. Reverse Phase HPLC was performed on either a Gilson system with a UV detector or an ACCQPrep system with UV and mass detection, equipped with ACE-5AQ, 100×21.2 mm, 5 μm columns. Compound analysis was performed by UPLC using an Agilent 1290 Infinity system (Methods listed below). LCMS analysis was performed using a Shimadzu LCMS-2020 system with PDA: SPD-M40 and MS: LCMS-2020 detectors using Kinetex EVO C₁₈ columns. The compounds prepared were named using IUPAC nomenclature.

### UPLC Methods

### Method A (5 Min, 5-100)

Phenomenex Kinetex XB-C18, 1.7 μm, 2.1×50 mm, 40° C., 0.8 mL/min, 5% MeCN (+0.1% formic acid) in water (+0.1% formic acid) for 0.7 min, 5-100% over 3.0 min, hold for 0.3 min, reequilibrate 1.0 min. 254 nm.

### Method B (5 Min, 5-100)

Phenomenex Kinetex XB-C18, 1.7 μm, 2.1×50 mm, 40° C., 0.8 mL/min, 5% MeCN (+0.1% TFA) in water (+0.1% TFA) for 1.0 min, 5-100% over 3.0 min, hold for 0.2 min, re-equilibrate 0.8 min. 200-300 nm.

### Method C (3 Min, 5-50)

HALO C18, 2.0 μm, 3.0×50 mm, 40° C., 1.5 mL/min, 5% MeCN (+0.1% formic acid) in water (+0.1% formic acid), 5-50% over 3.0 min. 254 nm.

### Method D (3 Min, 10-70)

Titan C18, 1.9 μm, 3.0×50 mm, 40° C., 1.5 mL/min, 10% MeCN in water (+0.04% NH₃·H₂O), 10-70% over 3.0 min. 254 nm.

### Method E (3 Min, 10-95)

Kinetex EVO C18, 2.6 μm, 3.0×50 mm, 40° C., 1.2 mL/min, 10% MeCN in water (5 mM NH₄HCO₃), 10-70% over 3.0 min. 254 nm.

### Method F (10 Min, 5-100)

Phenomenex Kinetex XB-C18, 1.7 μm, 2.1×100 mm, 40° C., 0.5 mL/min, 5% MeCN (+0.085% TFA) in water (+0.1% TFA) for 1.0 min, 5-100% over 8.0 min, hold for 0.2 min, re-equilibrate 0.8 min. 200-300 nm.

### Method G (3 Min, 5-50)

HALO C18, 2.0 μm, 3.0×50 mm, 40° C., 1.5 mL/min, 5% MeCN (+0.05% TFA) in water (+0.05% TFA), 5-95% over 3.0 min. 254 nm.

### EXPERIMENTAL PROCEDURES

### Intermediate 1

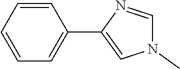

### 1-Methyl-4-phenyl-imidazole

Under an atmosphere of N₂, NaH (60% in mineral oil, 3.33 g, 83.2 mmol) was added to a solution of 4-phenyl-3H-imidazole (10 g, 69.4 mmol) in DMF (70 mL) at 0° C. The solution was stirred for 30 min before the dropwise addition of CH₃I (5.2 mL, 83.5 mmol) over 10 min while maintaining a temperature of 0° C. The resultant mixture was stirred for a further 1 h without cooling. The reaction was then quenched by the addition of water (100 mL) and extracted with EtOAc (5×100 mL). The combined organic layers were washed with brine (30 mL), dried over anhydrous Na₂SO₄, and concentrated in vacuo. The residue was purified by silica gel column chromatography to afford the title compound (7.80 g, 71.1%) as a yellow solid. LCMS (ES+): 159.0 [MH]⁺.

Intermediates 2-5 were prepared similarly to intermediate 1, by alkylation of an imidazole; see Table 1 below.

TABLE 1
Alkylation of imidazoles using sodium hydride

| | | |
|---|---|---|
| 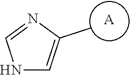 | 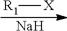 | 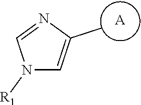 |

OR

| | | |
|---|---|---|
| 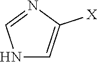 | 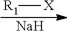 | 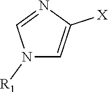 |

| | | | Intermediate, Form, |
|---|---|---|---|
| Int | Structure | Name | Yield, LCMS |
| 2 | 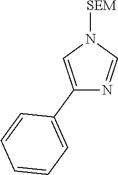 [SMILES: C[Si](C)(C)CCOCn1cnc(-c2ccccc2)c1] | Trimethyl-[2-[(4- phenylimidazol-1- yl)methoxy]ethyl] silane | From 4-phenyl-3H- imidazole and SEM-Cl Yellow Oil Yield 900 mg, 47.28% LCMS (ES+): 275.2 [MH]+. |
| 3 | 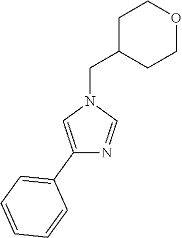 [SMILES: c1ccc(-c2cn(CC3CCOCC3)cn2)cc1] | 4-Phenyl-1- (tetrahydropyran-4- ylmethyl) imidazole | From 4-phenyl-3H- imidazole and 4-(2- bromoethyl)morpholine Orange oil Yield 500 mg, 44.5% LCMS (ES+): 243.2 [MH]+. |
| 4 | 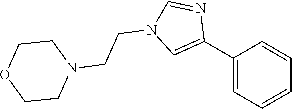 [SMILES: c1ccc(-c2cn(CCN3CCOCC3)cn2)cc1] | 4-[2-(4- Phenylimidazol-1- yl)ethyl] morpholine | From 4-phenyl-3H- imidazole and 4- (bromomethyl)oxane Yellow solid Yield 400 mg, 47.6% LCMS (ES+): 258.0 [MH]+. |
| 5 | 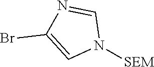 [SMILES: C[Si](C)(C)CCOCn1cnc(Br)c1] | 2-[(4- Bromoimidazol-1- yl)methoxy]ethyl- trimethyl-silane | From 4-bromo-1H- imidazole and SEM-Cl Brown solid Yield 3.12 g, 33.1% LCMS (ES+): 279.0 [MH]+. |

### Intermediate 6

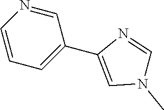

### 3-(1-Methylimidazol-4-yl)pyridine

Under an atmosphere of N₂, a mixture of 3-(4,4,5,5-tetramethyl-1,3,2-dioxaborolan-2-yl)pyridine (2.00 g, 9.80 mmol), 4-iodo-1-methylimidazole (2.28 g, 11.0 mmol), Pd(dppf)Cl₂—CH₂Cl₂ (0.81 g, 1.00 mmol) and K₂CO₃ (4.14 g, 30.0 mmol) in 1,4-dioxane (40 mL) and water (4.0 mL) was heated to 100° C. for 4 h. The reaction was concentrated in vacuo, diluted with water (10 mL) and extracted with EtOAc (3×10 mL). The combined organic layers were washed with brine (10 mL), dried over anhydrous Na₂SO₄, and concentrated in vacuo. The residue was purified by silica gel column chromatography to afford the title compound (590 mg, 38.0%) as a brown oil. LCMS (ES+): 160.2 [MH]⁺.

Intermediates 7 and 48 were prepared similarly to intermediate 6 via a Suzuki-Miyaura coupling; see Table 2 below.

TABLE 2
Suzuki-Miyaura couplings affording imidazoles

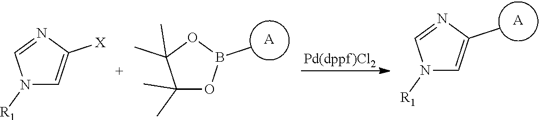

| | | | Intermediate, Form, |
|---|---|---|---|
| Int | Structure | Name | Yield, LCMS |
| 7 | 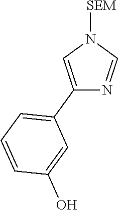 [SMILES: C[Si](C)(C)CCOCn1cnc(-c2cccc(O)c2)c1] | 3-[1-(2- Trimethylsilylethoxymethyl) imidazol-4- yl]phenol | From intermediate 5 and 3-(4,4,5,5- tetramethyl-1,3,2- dioxaborolan-2- yl)phenol Yellow solid Yield 3.1 g, 94.2% LCMS (ES+): 291.1 [MH]+. |
| 48 | 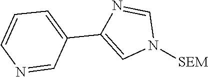 [SMILES: C[Si](C)(C)CCOCn1cnc(-c2cccnc2)c1] | 3-(1-((2- (Trimethylsilyl)ethoxy) methyl)-1H-imidazol-4- yl)pyridine | From intermediate 5 and 33-(4,4,5,5-tetramethyl- 1,3,2-dioxaborolan-2- yl)pyridine Yellow oil Yield 1.6 g, 80.5% LCMS (ES+): 276.2 [MH]+. |

### Intermediate 8

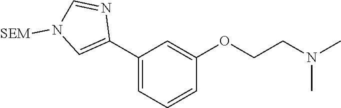 [SMILES: CN(C)CCOc1cccc(-c2cn(COCC[Si](C)(C)C)cn2)c1]

### N,N-dimethyl-2-[3-[1-(2-trimethylsilylethoxymethyl)imidazol-4-yl]phenoxy]ethanamine

Under an atmosphere of N₂, (2-bromoethyl)dimethylamine hydrobromide (2.69 g, 11.6 mmol) was added to a suspension of intermediate 7 (2.8 g, 9.64 mmol) and Cs₂CO₃ (6.28 g, 19.3 mmol) in DMF (30 mL). The resultant mixture was stirred at 100° C. for 16 h before quenching with water (50 mL) and extracting with EtOAc (5×50 mL). The combined organic layers were washed with brine (30 mL), dried over anhydrous Na₂SO₄, and concentrated in vacuo. The residue was purified by silica gel column chromatography to afford the title compound (570 mg, 16.0%) as a brown oil. LCMS (ES+): 362.2 [MH]⁺.

### Intermediate 9

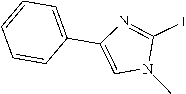

### 2-iodo-1-methyl-4-phenyl-imidazole

Under an atmosphere of N₂, n-BuLi (2.5 M in hexanes, 29.6 mL, 74.0 mmol) was added dropwise to a solution of intermediate 1 (7.8 g, 49.3 mmol) in THF (80 mL) over 10 min at −78° C. The solution was stirred for 1 h at −78° C. before the dropwise addition of I₂ (13.8 g, 54.2 mmol) in THF (20 mL) over 10 min. The resultant mixture was warmed to 0° C. and stirred for an additional 1 h before quenching with water (100 mL) and extracted with EtOAc (5×100 mL). The combined organic layers were washed with brine (30 mL), dried over anhydrous Na₂SO₄, and concentrated in vacuo. The residue was purified by silica gel column chromatography to afford the title compound (7.70 g, 55.0%) as a yellow solid. LCMS (ES+): 285.0 [MH]⁺.

Intermediates 10-14 and 49 were prepared similarly to intermediate 9, by iodination of an imidazole; see Table 3 below.

TABLE 3
Iodination of imidazoles using n-butyllithium and iodine

| | | |
|---|---|---|
| 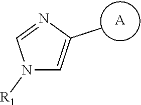 | 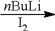 | 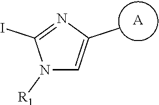 |

| | | | Intermediate, Form, |
|---|---|---|---|
| Int | Structure | Name | Yield, LCMS |
| 10 | 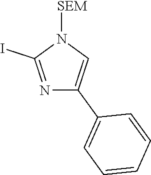 [SMILES: C[Si](C)(C)CCOCn1cc(-c2ccccc2)nc1I] | 2-[(2-lodo-4-phenyl- imidazol-1- yl)methoxy]ethyl- trimethyl-silane | From intermediate 2 Yellow Oil Yield 900 mg, 47.28% LCMS (ES+): 401.1 [MH]+. |
| 11 | 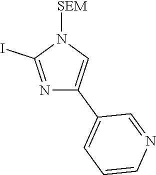 [SMILES: C[Si](C)(C)CCOCn1cc(-c2cccnc2)nc1I] | 3-(2-lodo-1-methyl- imidazol-4-yl)pyridine | From intermediate 6 Yellow solid Yield 150 mg, 27.9% LCMS (ES+): 286.0 [MH]+. |
| 49 | 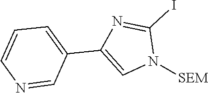 [SMILES: C[Si](C)(C)CCOCn1cc(-c2cccnc2)nc1I] | 3-(2-lodo-1-((2- (trimethylsilyl)ethoxy) methyl)-1H-imidazol- 4-yl)pyridine | From intermediate 48 Yellow solid Yield 1.0 g, 42.9% LCMS (ES+): 402.1 [MH]+. |
| 12 | 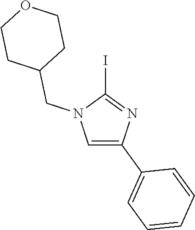 [SMILES: Ic1nc(-c2ccccc2)cn1CC1CCOCC1] | 2-lodo-4-phenyl-1- (tetrahydropyran-4- ylmethyl)imidazole | From intermediate 3 Yellow oil Yield 300 mg, 49.4% LCMS (ES+): 369.0 [MH]+. |
| 13 | 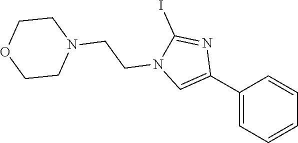 [SMILES: Ic1nc(-c2ccccc2)cn1CCN1CCOCC1] | 4-[2-(2-lodo-4-phenyl- imidazol-1- yl)ethyl]morpholine | From intermediate 4 Orange solid Yield 250 mg, 35.0% LCMS (ES+): 384.1 [MH]+. |
| 14 | 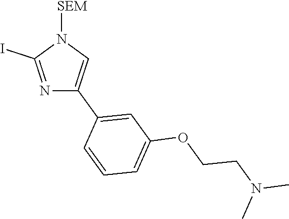 [SMILES: CN(C)CCOc1cccc(-c2cn(COCC[Si](C)(C)C)c(I)n2)c1] | 2-[3-[2-lodo-1-(2- trimethylsilylethoxymethyl) imidazol-4-yl]phenoxy]-N,N- dimethyl-ethanamine | From intermediate 8 Yellow oil Yield 491 mg, 63.7% LCMS (ES+): 488.2 [MH]+. |

### Intermediate 15

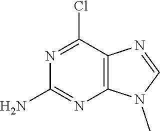

### 6-Chloro-9-methyl-purin-2-amine

Under an atmosphere of N₂, a suspension of 6-chloro-9H-purin-2-amine (15.0 g, 59.0 mmol), CH₃I (3.67 mL, 59.0 mmol) and K₂CO₃ (16.3 g, 118 mmol) in DMF (100 mL) was stirred for 16 h before concentrating in vacuo. The residue was purified by silica gel column chromatography to afford the title compound (3.4 g, 24.9%) as a yellow solid. LCMS (ES+): 184.0 [MH]⁺.

### Intermediate 50

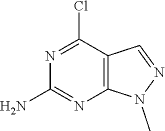

### 4-Chloro-1-methyl-1H-pyrazolo[3,4-d]pyrimidin-6-amine

Under and atmosphere of N₂, methylhydrazine sulfate (3.75 g, 26.0 mmol) was added to a solution of 2-amino-4,6-dichloropyrimidine-5-carbaldehyde (5.00 g, 26.0 mmol) and TEA (10.9 mL, 78.2 mmol) in DMF (50 mL). The resultant mixture was stirrer at 50° C. for 1 h before concentration in vacuo. The resultant residue purified by silica gel column chromatography to afford the title compound (3.82 g, 79.9%) as a yellow solid. LCMS (ES+): 184.1 [MH]⁺.

Intermediate 51 were prepared similarly to intermediate 50, by pyrazole formation via cyclisation; see Table 12 below.

TABLE 12
Pyrazole formation via cyclisation

| | |
|---|---|
| 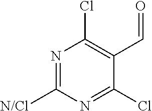 | 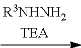 |

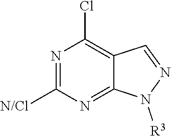

| | | | Intermediate, | |
|---|---|---|---|---|
| Int | Structure | Name | Form, LCMS | Yield, |
| 51 | 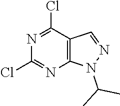 | 4,6- Dichloro-1- isopropyl- 1H- pyrazolo [3,4-d] pyrimidine | From trichloropyrimidine- 5-carbaldehyde isopropylhydrazine hydrochloride Yellow Solid Yield 2.40 g, 231.2% | 2,4,6- and [MH]+. |
| | | | LCMS (ES+): 231.2 | |

### Intermediate 16

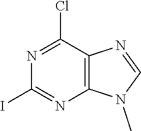

### 6-Chloro-2-iodo-9-methyl-purine

Under an atmosphere of N₂, isoamyl nitrite (7.47 mL, 55.6 mmol) was added to a suspension of intermediate 15 (3.4 g, 18.5 mmol, 1.0 equiv), CuI (3.53 g, 18.5 mmol), and CH₂I₂ (7.47 mL, 92.6 mmol) in THF (70 mL) and the reaction was heated to 80° C. for 2 h. The reaction was then concentrated in vacuo and the resultant residue purified by silica gel column chromatography to afford the title compound (3.2 g, 58.7%) as a yellow solid. LCMS (ES+): 295.1 [MH]⁺.

Intermediate 52 were prepared similarly to intermediate 16, by iodination of pyrimidines via Sandmeyer reaction; see Table 13 below.

TABLE 13
lodination of pyrimidines via Sandmeyer reaction

| | |
|---|---|
| 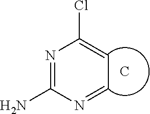 | 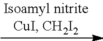 |
| | 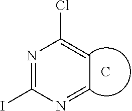 |

| | | | Intermediate, Form, |
|---|---|---|---|
| Int | Structure | Name | Yield, LCMS |
| 52 | 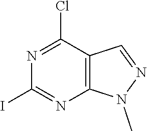 | 4-Chloro- 6- iodo-1- methyl- 1H- pyrazolo [3,4-d] pyrimidine | From intermediate 50 Yellow Solid Yield 3.10 g, 50.1% LCMS (ES+): 295.0 [MH]+. |

### Intermediate 17

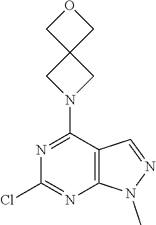 [SMILES: Cn1ncc2c(N3CC4(COC4)C3)nc(Cl)nc21]

### 6-(6-Chloro-1-methyl-pyrazolo[3,4-d]pyrimidin-4-yl)-2-oxa-6-azaspiro[3.3]heptane

A solution of 4,6-dichloro-1-methylpyrazolo[3,4-d]pyrimidine (3.00 g, 14.9 mmol), 2-oxa-6-azaspiro[3.3]heptane (1.47 g, 14.9 mmol), and DIPEA (7.76 mL, 44.6 mmol) in EtOH (30 mL) was stirred for 16 h before concentration in vacuo. The resultant residue was washed with PE (3×5 mL) and dried under vacuum affording the title compound (1.92 g, 48.8%) as a yellow solid. The material was taken into the next reaction without any further purification. LCMS (ES+): 266.1 [MH]⁺.

Intermediates 18-23 and 53-55 were prepared similarly to intermediate 17, by SnAr of a halogenated pyrimidines; see Table 4 below.

TABLE 4
SnAr of halogenated pyrimidines

| | | |
|---|---|---|
| 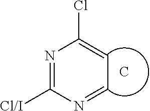 | 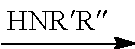 | 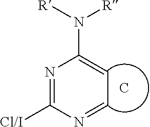 |

| | | | Intermediate, Form, Yield, |
|---|---|---|---|
| Int | Structure | Name | LCMS |
| 18 | 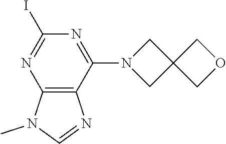 [SMILES: Cn1cnc2c(N3CC4(COC4)C3)nc(I)nc21] | 2-iodo-9-methyl-6- (2- oxaspiro[3.3]heptan- 6-yl)purine | From intermediate 16 and 2- oxa-6-azaspiro[3.3]heptane Yellow Solid Yield 2.20 g, 56.7% LCMS (ES+): 357.8 [MH]+. |
| 19 | 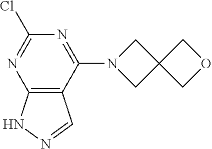 [SMILES: Clc1nc(N2CC3(COC3)C2)c2cn[nH]c2n1] | 6-(6-Chloro-1H- pyrazolo[3,4- d]pyrimidin-4-yl)-2- oxa-6- azaspiro[3.3]heptane | From 4,6-dichloro-1H- pyrazolo[3,4-d]pyrimidine and 2-oxa-6-azaspiro[3.3]heptane Yellow solid Yield 6.96 g, 52.1% LCMS (ES+): 252.1 [MH]+. |
| 53 | 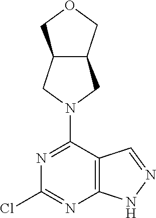 [SMILES: Clc1nc(N2C[C@H]3COC[C@H]3C2)c2cn[nH]c2n1] | (3aR,6aS)-5-(6- Chloro-1H- pyrazolo[3,4- d]pyrimidin-4- yl)hexahydro-1H- furo[3,4-c]pyrrole | From ,6-dichloro-1H- pyrazolo[3,4-d]pyrimidine and (3aR,6aS)-hexahydro-1H- furo[3,4-c]pyrrole White solid Yield 4.2 g, 59.8% LCMS (ES+): 266.1 [MH]+. |
| 54 | 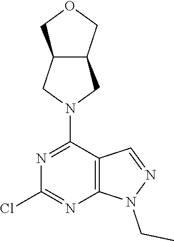 [SMILES: CCn1ncc2c(N3C[C@H]4COC[C@H]4C3)nc(Cl)nc21] | (3aR,6aS)-5-(6- Chloro-1-ethyl-1H- pyrazolo[3,4- d]pyrimidin-4- yl)hexahydro-1H- furo[3,4-c]pyrrole | From 4,6-dichloro-1- ethylpyrazolo[3,4- d]pyrimidine and (3aR,6aS)- hexahydro-1H-furo[3,4- c]pyrrole White solid Yield 1.10 g, 81.3% LCMS (ES+): 294.2 [MH]+. |
| 20 | 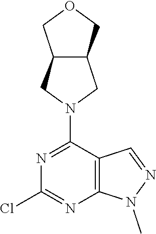 [SMILES: Cn1ncc2c(N3C[C@H]4COC[C@H]4C3)nc(Cl)nc21] | (3aS,6aR)-5-(6- chloro-1-methyl- pyrazolo[3,4- d]pyrimidin-4-yl)- 1,3,3a,4,6,6a- hexahydrofuro[3,4- c]pyrrole | From 4,6-dichloro-1- methylpyrazolo[3,4- d]pyrimidine and (3aR,6aS)- hexahydro-1H-furo[3,4- c]pyrrole Yellow solid Yield 1.50 g, 72.6% LCMS (ES+): 280.1 [MH]+. |
| 55 | 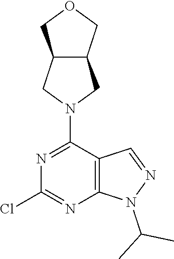 [SMILES: CC(C)n1ncc2c(N3C[C@H]4COC[C@H]4C3)nc(Cl)nc21] | (3aR,6aS)-5-(6- Chloro-1-isopropyl- 1H-pyrazolo[3,4- d]pyrimidin-4- yl)hexahydro-1H- furo[3,4-c]pyrrole | From intermediate 51 and (3aR,6aS)-hexahydro-1H- furo[3,4-c]pyrrole Yellow solid Yield 1.10 g, 82.5% LCMS (ES+): 308.2 [MH]+. |
| 21 | 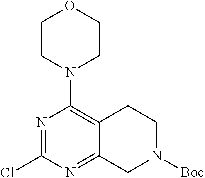 [SMILES: CC(C)(C)OC(=O)N1CCc2c(nc(Cl)nc2N2CCOCC2)C1] | tert-Butyl 2-chloro- 4-morpholino-6,8- dihydro-5H- pyrido[3,4- d]pyrimidine-7- carboxylate | From tert-butyl 2,4-dichloro- 5H,6H,8H-pyrido[3,4- d]pyrimidine-7-carboxylate and morpholine White solid Yield 4.90 g, 93.2% LCMS (ES+): 355.2 [MH]+. |
| 22 | 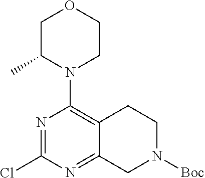 [SMILES: C[C@@H]1COCCN1c1nc(Cl)nc2c1CCN(C(=O)OC(C)(C)C)C2] | tert-Butyl 2-chloro- 4-[(3R)-3- methylmorpholin- 4-yl]-6,8-dihydro- 5H-pyrido[3,4- d]pyrimidine-7- carboxylate | From tert-butyl 2,4-dichloro- 5H,6H,8H-pyrido[3,4- d]pyrimidine-7-carboxylate and (3R)-3-methylmorpholine Yellow Solid Yield 1.00 g, 41.2% LCMS (ES+): 369.2 [MH]+. |
| 23 | 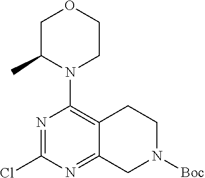 [SMILES: C[C@H]1COCCN1c1nc(Cl)nc2c1CCN(C(=O)OC(C)(C)C)C2] | tert-Butyl 2-chloro- 4-[(3S)-3- methylmorpholin- 4-yl]-6,8-dihydro- 5H-pyrido[3,4- d]pyrimidine-7- carboxylate | From tert-butyl 2,4-dichloro- 5H,6H,8H-pyrido[3,4- d]pyrimidine-7-carboxylate and (3S)-3-methylmorpholine Yellow Solid Yield 650 mg, 35.7% LCMS (ES+): 369.2 [MH]+. |

### Intermediate 24

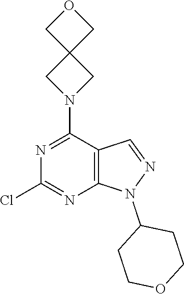 [SMILES: Clc1nc(N2CC3(COC3)C2)c2cnn(C3CCOCC3)c2n1]

### 6-(6-Chloro-1-tetrahydropyran-4-yl-pyrazolo[3,4-d]pyrimidin-4-yl)-2-oxa-6-azaspiro[3.3]heptane

Under an atmosphere of N₂, NaH (60% in mineral oil, 239 mg, 5.98 mmol) and 4-bromooxane (784 mg, 4.78 mmol) were sequentially added to solution of intermediate 19 (1.00 g, 3.98 mmol) in DMF (10 mL). The reaction was then heated to 100° C. for 5 h before quenching with water (10 mL) and then extracted with EtOAc (3×20 mL). The combined organic layers were washed with brine (20 mL), dried over anhydrous Na₂SO₄, and concentrated in vacuo. The residue was purified by silica gel column chromatography to afford the title compound (400 mg, 30.0%) as a yellow solid. LCMS (ES+): 336.2 [MH]⁺.

### Intermediate 25

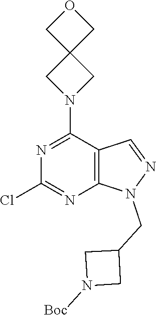 [SMILES: CC(C)(C)OC(=O)N1CC(Cn2ncc3c(N4CC5(COC5)C4)nc(Cl)nc32)C1]

### 6 tert-Butyl 3-[[6-chloro-4-(2-oxa-6-azaspiro[3.3]heptan-6-yl)pyrazolo[3,4-d]pyrimidin-1-yl]methyl]azetidine-1-carboxylate

3-(Iodomethyl)azetidine (1.17 g, 5.96 mmol) was added to a suspension of intermediate 19 (1.00 g, 3.97 mmol), Cs₂CO₃ (2.59 g, 7.95 mmol) in DMF (15 mL). The reaction was then heated to 80° C. for 2 h before quenching with water (20 mL) and then extracted with DCM (3×20 mL). The combined organic layers were washed with brine (10 mL), dried over anhydrous Na₂SO₄, and concentrated in vacuo. The residue was purified by silica gel column chromatography to afford the title compound (660 mg, 51.8%) as a yellow solid. LCMS (ES+): 421.2 [MH]⁺.

Intermediates 56 and 57 were prepared similarly to intermediate 25, by the alkylation of a pyrazolopyrimidine with Cs₂CO₃; see Table 14 below.

TABLE 14
Alkylation of pyrazolopyrimidines using Cs₂CO₃

| | | |
|---|---|---|
| 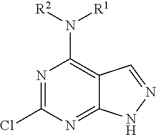 | 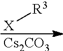 | 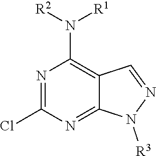 |

| | | | Intermediate, Form, Yield, |
|---|---|---|---|
| Int | Structure | Name | LCMS |
| 56 | 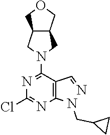 [SMILES: Clc1nc(N2C[C@H]3COC[C@H]3C2)c2cnn(CC3CC3)c2n1] | (3aR,6aS)-5-(6- Chloro-1- (cyclopropylmethyl)- 1H-pyrazolo[3,4- d]pyrimidin-4- yl)hexahydro-1H- furo[3,4-c]pyrrole | From intermediate 53 and (iodomethyl)cyclopropane Yellow Solid Yield 320 mg, 44.3% LCMS (ES+): 322.0 [MH]+. |
| 57 | 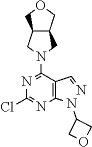 [SMILES: Clc1nc(N2C[C@H]3COC[C@H]3C2)c2cnn(C3COC3)c2n1] | (3aR,6aS)-5-(6- Chloro-1-(oxetan-3- yl)-1H-pyrazolo[3,4- d]pyrimidin-4- yl)hexahydro-1H- furo[3,4-c]pyrrole | From intermediate 53 and 3- iodooxetane Yellow Solid Yield 3.50 g, 68.8% LCMS (ES+): 322.1 [MH]+. |

### Intermediate 26

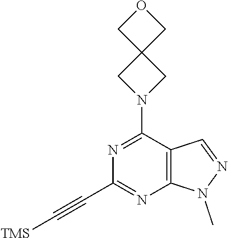 [SMILES: Cn1ncc2c(N3CC4(COC4)C3)nc(C#C[Si](C)(C)C)nc21]

### Trimethyl-[2-[1-methyl-4-(2-oxa-6-azaspiro[3.3]heptan-6-yl)pyrazolo[3,4-d]pyrimidin-6-yl]ethynyl]silane

Under an atmosphere of N₂, TEA (996 μL, 7.15 mmol) and trimethylsilylacetylene (848 μL, 5.96 mmol) were sequentially added dropwise to a solution of intermediate 17 (600 mg, 2.26 mmol), CuI (90.8 mg, 0.48 mmol), and Pd(dppf)Cl₂—CH₂Cl₂ (583 mg, 0.72 mmol) in DMF (25 mL) over 10 min. The resultant mixture was stirred for 1 h before dilution with water (15 mL) and extracted with DCM (3×15 mL). The combined organic layers were washed with brine (20 mL), dried over anhydrous Na₂SO₄, and concentrated in vacuo. The residue was purified by silica gel column chromatography to afford the title compound (625 mg, 84.5%) as a yellow solid. LCMS (ES+): 328.3 [MH]⁺.

Intermediates 27-33 and 58-63 were prepared similarly to intermediate 17, via Sonogashira coupling with trimethylsilylacetylene; see Table 5 below.

TABLE 5
Sonogashira Couplings affording TMS-alkynes

| | | |
|---|---|---|
| 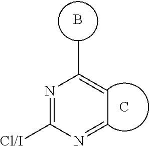 | 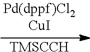 | 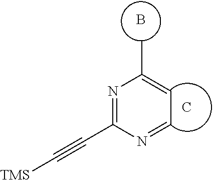 |

| | | | Intermediate, Form, |
|---|---|---|---|
| Int | Structure | Name | Yield, LCMS |
| 58 | 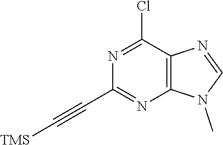 [SMILES: Cn1cnc2c(Cl)nc(C#C[Si](C)(C)C)nc21] | 6-Chloro-9-methyl-2- ((trimethylsilyl)ethynyl)- 9H-purine | From intermediate 16 Yellow solid Yield 3.00 g, 74.1% LCMS (ES+): 265.2 [MH]+. |
| 59 | 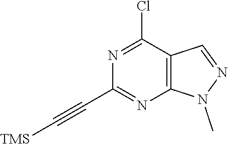 [SMILES: Cn1ncc2c(Cl)nc(C#C[Si](C)(C)C)nc21] | 4-Chloro-1-methyl-6- ((trimethylsilyl)ethynyl)- 1H-pyrazolo[3,4- d]pyrimidine | From intermediate 52 Brown solid Yield 1.1 g, 61.2% LCMS (ES+): 265.2 [MH]+. |
| 27 | 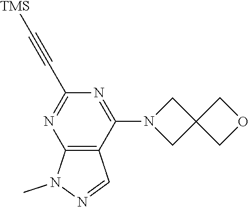 [SMILES: Cn1ncc2c(N3CC4(COC4)C3)nc(C#C[Si](C)(C)C)nc21] | Trimethyl-[2-[9-methyl-6- (2-oxa-6- azaspiro[3.3]heptan-6- yl)purin-2- yl]ethynyl]silane | From intermediate 18 Brown solid Yield 1.50 g, 74.4% LCMS (ES+): 328.1 [MH]+. |
| 28 | 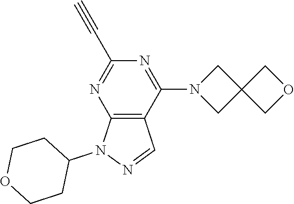 [SMILES: C#Cc1nc(N2CC3(COC3)C2)c2cnn(C3CCOCC3)c2n1] | 6-(6-Ethynyl-1- tetrahydropyran-4-yl- pyrazolo[3,4-d]pyrimidin- 4-yl)-2-oxa-6- azaspiro[3.3]heptane Note: The TMS group was cleaved in situ under these reaction conditions | From intermediate 24 White solid Yield 60 mg, 17.2% LCMS (ES+): 326.3 [MH]+. |
| 29 | 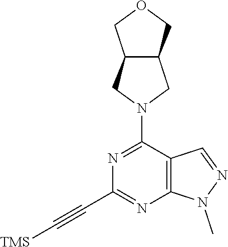 [SMILES: Cn1ncc2c(N3C[C@H]4COC[C@H]4C3)nc(C#C[Si](C)(C)C)nc21] | 2-[4-[(3aS,6aR)- 1,3,3a,4,6,6a- hexahydrofuro[3,4- c]pyrrol-5-yl]-1-methyl- pyrazolo[3,4-d]pyrimidin- 6-yl]ethynyl-trimethyl- silane | From intermediate 20 Yellow solid Yield 1.50 g, 81.9% LCMS (ES+): 342.2 [MH]+. |
| 60 | 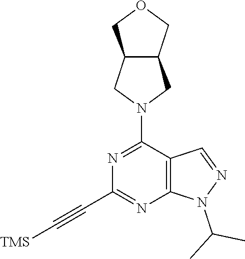 [SMILES: CC(C)n1ncc2c(N3C[C@H]4COC[C@H]4C3)nc(C#C[Si](C)(C)C)nc21] | (3aR,6aS)-5-(1-isoPropyl- 6-((trimethylsilyl)ethynyl)- 1H-pyrazolo[3,4- d]pyrimidin-4- yl)hexahydro-1H-furo[3,4- c]pyrrole | From intermediate 55 Yellow solid Yield 1.06 g, 80.2% LCMS (ES+): 370.2 [MH]+. |
| 61 | 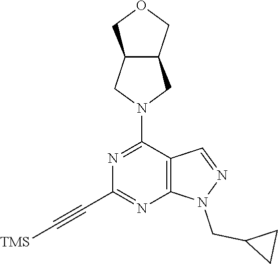 [SMILES: C[Si](C)(C)C#Cc1nc(N2C[C@H]3COC[C@H]3C2)c2cnn(CC3CC3)c2n1] | (3aR,6aS)-5-(1- (Cyclopropylmethyl)-6- ((trimethylsilyl)ethynyl)- 1H-pyrazolo[3,4- d]pyrimidin-4- yl)hexahydro-1H-furo[3,4- c]pyrrole | From intermediate 56 Yellow solid Yield 300 mg, 78.5% LCMS (ES+): 382.1 [MH]+. |
| 62 | 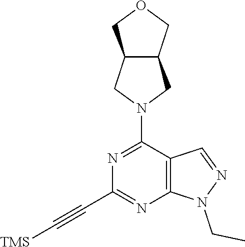 [SMILES: CCn1ncc2c(N3C[C@H]4COC[C@H]4C3)nc(C#C[Si](C)(C)C)nc21] | (3aR,6aS)-5-(1-ethyl-6- ((Trimethylsilyl)ethynyl)- 1H-pyrazolo[3,4- d]pyrimidin-4- yl)hexahydro-1H-furo[3,4- c]pyrrole | From intermediate 54 White solid Yield 1.10 g, 82.6% LCMS (ES+): 356.4 [MH]+. |
| 63 | 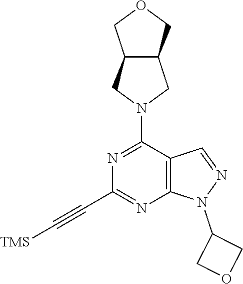 [SMILES: C[Si](C)(C)C#Cc1nc(N2C[C@H]3COC[C@H]3C2)c2cnn(C3COC3)c2n1] | (3aR,6aS)-5-(1-(Oxetan-3- yl)-6- ((Trimethylsilyl)ethynyl)- 1H-pyrazolo[3,4- d]pyrimidin-4- yl)hexahydro-1H-furo[3,4- c]pyrrole | From intermediate 57 Yellow solid Yield 1.20 g, 50.3% LCMS (ES+): 384.2 [MH]+. |
| 30 | 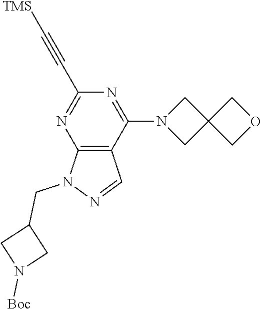 [SMILES: CC(C)(C)OC(=O)N1CC(Cn2ncc3c(N4CC5(COC5)C4)nc(C#C[Si](C)(C)C)nc32)C1] | tert-Butyl 3-[[4-(2-oxa-6- azaspiro[3.3]heptan-6-yl)- 6-(2- trimethylsilylethynyl) pyrazolo[3,4-d]pyrimidin-1- yl]methyl]azetidine-1- carboxylate | From intermediate 25 Brown solid Yield 345 mg, 46.3% LCMS (ES+): 483.2 [MH]+. |
| 31 | 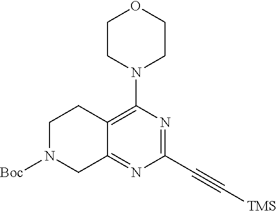 [SMILES: CC(C)(C)OC(=O)N1CCc2c(nc(C#C[Si](C)(C)C)nc2N2CCOCC2)C1] | tert-Butyl 4-morpholino-2- (2-trimethylsilylethynyl)- 6,8-dihydro-5H-pyrido[3,4- d]pyrimidine-7- carboxylate | From intermediate 21 Brown solid Yield 2.00 g, 85.1% LCMS (ES+): 417.3 [MH]+. |
| 32 | 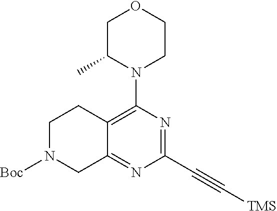 [SMILES: C[C@@H]1COCCN1c1nc(C#C[Si](C)(C)C)nc2c1CCN(C(=O)OC(C)(C)C)C2] | tert-Butyl 4-[(3R)-3- methylmorpholin-4-yl]-2- (2-trimethylsilylethynyl)- 6,8-dihydro-5H-pyrido[3,4- d]pyrimidine-7- carboxylate | From intermediate 22 Yellow solid Yield 0.90 g, 77.1% LCMS (ES+): 431.2 [MH]+. |
| 33 | 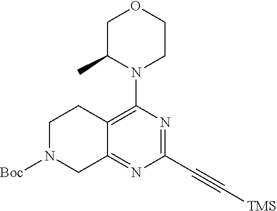 [SMILES: C[C@H]1COCCN1c1nc(C#C[Si](C)(C)C)nc2c1CCN(C(=O)OC(C)(C)C)C2] | tert-Butyl 4-[(3S)-3- methylmorpholin-4-yl]-2- (2-trimethylsilylethynyl)- 6,8-dihydro-5H-pyrido[3,4- d]pyrimidine-7- carboxylate | From intermediate 23 Brown solid Yield 500 mg, 65.9% LCMS (ES+): 431.2 [MH]+. |

### Intermediate 34

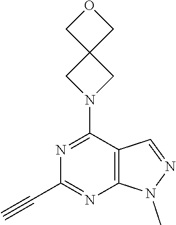 [SMILES: C#Cc1nc(N2CC3(COC3)C2)c2cnn(C)c2n1]

### 6-(6-Ethynyl-1-methyl-pyrazolo[3,4-d]pyrimidin-4-yl)-2-oxa-6-azaspiro[3.3]heptane

A suspension of intermediate 26 (625 mg, 1.91 mmol) and K₂CO₃ (551 mg, 3.99 mmol) in MeOH (5.0 mL) and DCM (5.0 mL) was stirred for 1 h before being dilution with water (15 mL) and extraction with DCM (3×15 mL). The combined organic layers were washed with brine (20 mL), dried over anhydrous Na₂SO₄, and concentrated in vacuo. The residue was purified by silica gel column chromatography to afford the title compound (380 mg, 78.0%) as a yellow solid. LCMS (ES+): 256.1 [MH]⁺.

Intermediates 35-40 and 64-69 were prepared similarly to intermediate 34, via removal of an alkynyl TMS group; see Table 6 below.

TABLE 6
alkynyl TMS deprotection

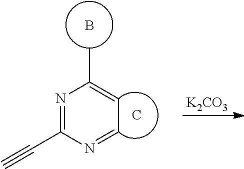

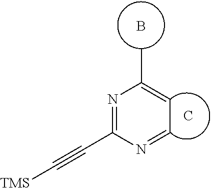

| | | | Intermediate, Form, |
|---|---|---|---|
| Int | Structure | Name | Yield, LCMS |
| 64 | 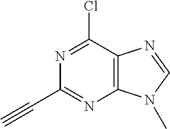 | 6-Chloro-2-ethynyl-9- methyl-9H-purine | From intermediate 58 Yellow solid Yield 1.80 g, 57.6% LCMS (ES+): 193.2 [MH]+. |
| 65 | 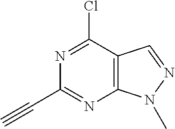 | 4-Chloro-6-ethynyl-1- methyl-1H-pyrazolo[3,4- d]pyrimidine | From intermediate 59 Yellow solid Yield 530 mg, 65.2% LCMS (ES+): 193.1 [MH]+. |
| 35 | 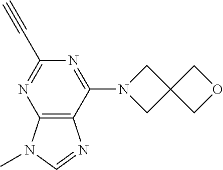 [SMILES: C#Cc1nc(N2CC3(COC3)C2)c2ncn(C)c2n1] | 6-(2-Ethynyl-9-methyl- purin-6-yl)-2-oxa-6- azaspiro[3.3]heptane | From intermediate 27 Brown solid Yield 700 mg, 51.6% LCMS (ES+): 256.1 [MH]+. |
| 36 | 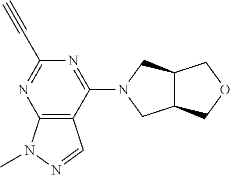 [SMILES: C#Cc1nc(N2C[C@H]3COC[C@H]3C2)c2cnn(C)c2n1] | (3aS,6aR)-5-(6-ethynyl-1- methyl-pyrazolo[3,4- d]pyrimidin-4-yl)- 1,3,3a,4,6,6a- hexahydrofuro[3,4- c]pyrrole | From intermediate 29 Yellow solid Yield 590 mg, 49.9% LCMS (ES+): 270.1 [MH]+. |
| 66 | 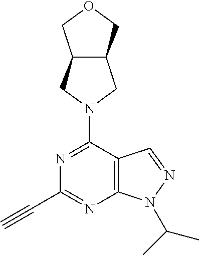 [SMILES: C#Cc1nc(N2C[C@H]3COC[C@H]3C2)c2cnn(C(C)C)c2n1] | (3aR,6aS)-5-(6-Ethynyl-1- isopropyl-1H-pyrazolo[3,4- d]pyrimidin-4- yl)hexahydro-1H-furo[3,4- c]pyrrole | From intermediate 60 White solid Yield 700 mg, 82.1% LCMS (ES+): 298.2 [MH]+. |
| 67 | 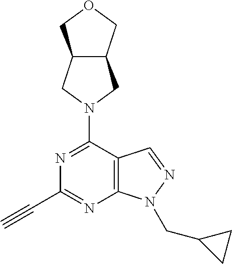 [SMILES: C#Cc1nc(N2C[C@H]3COC[C@H]3C2)c2cnn(CC3CC3)c2n1] | (3aR,6aS)-5-(1- (Cyclopropylmethyl)-6- ethynyl-1H-pyrazolo[3,4- d]pyrimidin-4- yl)hexahydro-1H-furo[3,4- c]pyrrole | From intermediate 61 Yellow solid Yield 230 mg, 94.6% LCMS (ES+): 310.3 [MH]+. |
| 68 | 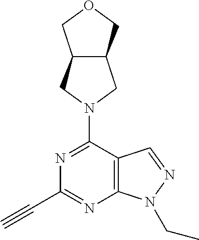 [SMILES: C#Cc1nc(N2C[C@H]3COC[C@H]3C2)c2cnn(CC)c2n1] | (3aR,6aS)-5-(1-Ethyl-6- ethynyl-1H-pyrazolo[3,4- d]pyrimidin-4- yl)hexahydro-1H-furo[3,4- c]pyrrole | From intermediate 62 White solid Yield 700 mg, 79.8% LCMS (ES+): 284.2 [MH]+. |
| 69 | 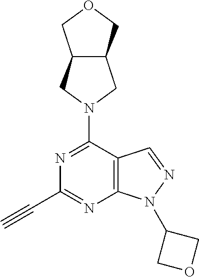 [SMILES: C#Cc1nc(N2C[C@H]3COC[C@H]3C2)c2cnn(C3COC3)c2n1] | (3aR,6aS)-5-(6-Ethynyl-1- (oxetan-3-yl)-1H- pyrazolo[3,4-d]pyrimidin-4- yl)hexahydro-1H-furo[3,4- c]pyrrole | From intermediate 63 White solid Yield 650 mg, 66.7% LCMS (ES+): 312.2 [MH]+. |
| 37 | 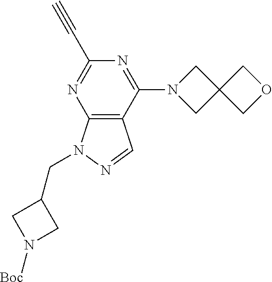 [SMILES: C#Cc1nc(N2CC3(COC3)C2)c2cnn(CC3CN(C(=O)OC(C)(C)C)C3)c2n1] | tert-Butyl 3-[[6-ethynyl-4-(2- oxa-6-azaspiro[3.3]heptan- 6-yl)pyrazolo[3,4- d]pyrimidin-1- yl]methyl]azetidine-1- carboxylate | From intermediate 30 Used without purification |
| 38 | 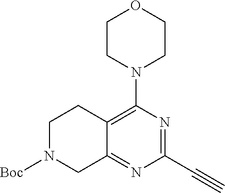 [SMILES: C#Cc1nc2c(c(N3CCOCC3)n1)CCN(C(=O)OC(C)(C)C)C2] | tert-Butyl 2-ethynyl-4- morpholino-6,8-dihydro-5H- pyrido[3,4-d]pyrimidine-7- carboxylate | From intermediate 31 Yellow Solid Yield 700 mg, 42.4% LCMS (ES+): 345.2 [MH]+. |
| 39 | 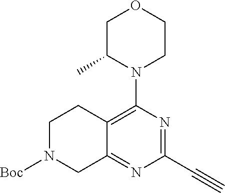 [SMILES: C#Cc1nc2c(c(N3CCOC[C@H]3C)n1)CCN(C(=O)OC(C)(C)C)C2] | tert-Butyl 2-ethynyl-4-[(3R)- 3-methylmorpholin-4-yl]- 6,8-dihydro-5H-pyrido[3,4- d]pyrimidine-7-carboxylate | From intermediate 32 Yellow Solid Yield 500 mg, 54.6% LCMS (ES+): 359.2 [MH]+. |
| 40 | 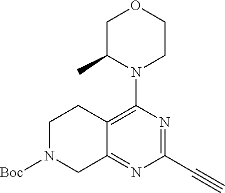 [SMILES: C#Cc1nc2c(c(N3CCOC[C@@H]3C)n1)CCN(C(=O)OC(C)(C)C)C2] | tert-Butyl 2-ethynyl-4-[(3S)- 3-methylmorpholin-4-yl]- 6,8-dihydro-5H-pyrido[3,4- d]pyrimidine-7-carboxylate | From intermediate 33 Yellow Solid Yield 350 mg, 70.1% LCMS (ES+): 359.2 [MH]+. |

### Intermediate 41

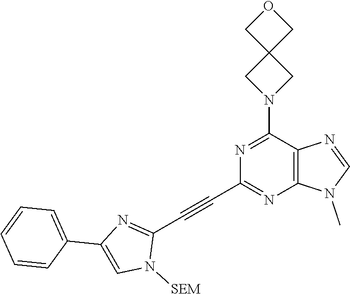 [SMILES: Cn1cnc2c(N3CC4(COC4)C3)nc(C#Cc3nc(-c4ccccc4)cn3COCC[Si](C)(C)C)nc21]

### Trimethyl-[2-[[2-[2-[9-methyl-6-(2-oxa-6-azaspiro[3.3]heptan-6-yl)purin-2-yl]ethynyl]-4-phenyl-imidazol-1-yl]methoxy]ethyl]silane

Under an atmosphere of N₂, a solution of Pd(PPh₃)₂Cl₂ (27.5 mg, 39.0 μmol) and intermediate 35 (78.4 mg, 196 μmol) in THF (5 mL) was added dropwise to a suspension of intermediate 10 (50.0 mg, 196 μmol), Cs₂CO₃ (128 mg, 392 μmol) and CuI (14.9 mg, 78 μmol) in THF (10 mL) over 10 min. The resultant mixture was stirred for 2 h before diluting with water (5 mL) and extracted with EtOAc (3×15 mL). The combined organic layers were washed with brine (10 mL), dried over anhydrous Na₂SO₄, and concentrated in vacuo. The residue was purified by silica gel column chromatography to afford the title compound (45.0 mg, 43.5%) as a yellow solid. LCMS (ES+): 527.6 [M]⁺.

Intermediate 42 and examples 1-4 were prepared similarly to intermediate 41, via Sonogashira coupling using Pd(PPh₃)₂Cl₂; see Table 7 below (which is split in two parts).

TABLE 7
Sonogashira Coupling with Pd(PPh₃)₂Cl₂

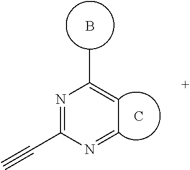

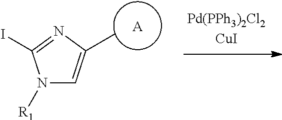

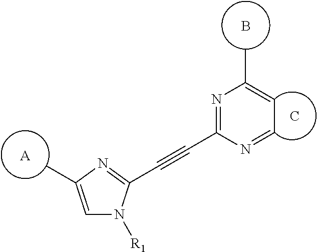

| | | | Intermediate, Form, |
|---|---|---|---|
| Int | Structure | Name | Yield, LCMS |
| 42 | 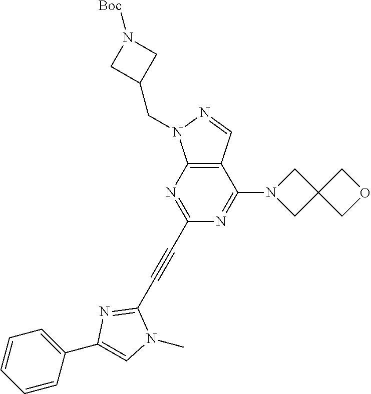 [SMILES: Cn1cc(-c2ccccc2)nc1C#Cc1nc(N2CC3(COC3)C2)c2cnn(CC3CN(C(=O)OC(C)(C)C)C3)c2n1] | tert-Butyl 3-[6-[2-(1- methyl-4-phenyl- imidazol-2-yl)ethynyl]- 4-(2-oxa-6- azaspiro [3.3]heptan-6- yl)pyrazolo[3,4- d]pyrimidin-1- yl]methyl]azetidine-1- carboxylate | From intermediates 9 and 37 Yellow solid Yield 80 mg, 36.5% LCMS (ES+): 567.3 [MH]+. |
| 1 | 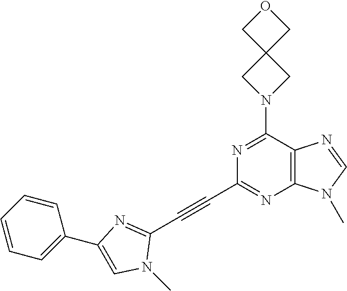 [SMILES: Cn1cc(-c2ccccc2)nc1C#Cc1nc(N2CC3(COC3)C2)c2ncn(C)c2n1] | 6-[9-Methyl-2-[2-(1- methyl-4-phenyl- imidazol-2- yl)ethynyl]purin-6-yl]- 2-oxa-6- azaspiro[3.3]heptane | From intermediates 9 and 35 Yellow solid Yield 8.0 mg, 24.0% UPLC (Method A): Rt 1.91 min. HRMS (ES+/QToF) m/z: [M + H]⁺ Calcd for C₂₃H₂₂N₇O 412.1886; Found 412.1886. |
| 2 | 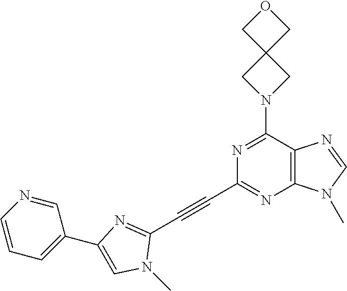 [SMILES: Cn1cc(-c2cccnc2)nc1C#Cc1nc(N2CC3(COC3)C2)c2ncn(C)c2n1] | 6-[9-Methyl-2-[2-[1- methyl-4-(3- pyridyl)imidazol-2- yl]ethynyl]purin-6-yl]- 2-oxa-6- azaspiro[3.3]heptane | From intermediates 11 and 35 Brown solid Yield 85.6 mg, 39.5% UPLC (Method A): Rt 1.45 min. HRMS (ES+/QToF) m/z: [M + H]⁺ Calcd for C₂₂H₂₁N₈O 413.1838; Found 413.1841. |
| 3 | 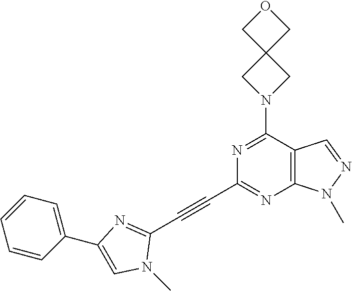 [SMILES: Cn1cc(-c2ccccc2)nc1C#Cc1nc(N2CC3(COC3)C2)c2cnn(C)c2n1] | 6-[1-Methyl-6-[2-(1- methyl-4-phenyl- imidazol-2- yl)ethynyl]pyrazolo[3, 4-d]pyrimidin-4-yl]-2- oxa-6- azaspiro[3.3]heptane | From intermediates 9 and 34 White solid Yield 14.5 mg, 10.0% UPLC (Method A): Rt 1.99 min. HRMS (ES+/QToF) m/z: [M + H]⁺ Calcd for C₂₃H₂₂N₇O 412.1886; Found 412.1885. |
| 4 | 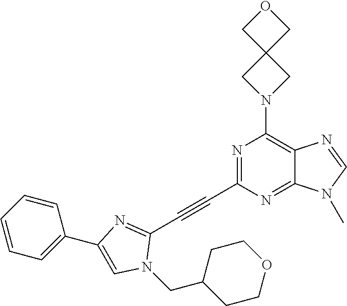 [SMILES: Cn1cnc2c(N3CC4(COC4)C3)nc(C#Cc3nc(-c4ccccc4)cn3CC3CCOCC3)nc21] | 6-[9-Methyl-2-[2-[4- phenyl-1- (tetrahydropyran-4- ylmethyl)imidazol-2- yl]ethynyl]purin-6-yl]- 2-oxa-6- azaspiro[3.3]heptane | From intermediates 12 and 35 Yellow solid Yield 163 mg, 41.1% UPLC (Method A): Rt 2.07 min. HRMS (ES+/QToF) m/z: [M + H]⁺ Calcd for C₂₈H₃₀N₇O₂ 496.2461; Found 496.2462. |
| 5 | 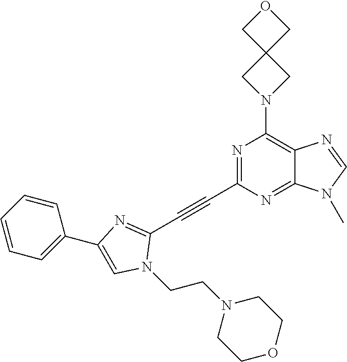 [SMILES: Cn1cnc2c(N3CC4(COC4)C3)nc(C#Cc3nc(-c4ccccc4)cn3CCN3CCOCC3)nc21] | 6-[9-Methyl-2-[2-[1-(2- morpholinoethyl)-4- phenyl-imidazol-2- yl]ethynyl]purin-6-yl]- 2-oxa-6- azaspiro[3.3]heptane | From intermediates 13 and 35 Yellow Solid Yield 58.9 mg, 31.3% UPLC (Method A): Rt 1.58 min. HRMS (ES+/QToF) m/z: [M + H]⁺ Calcd for C₂₈H₃₁N₈O₂ 511.2570; Found 511.2567. |

### Intermediate 43

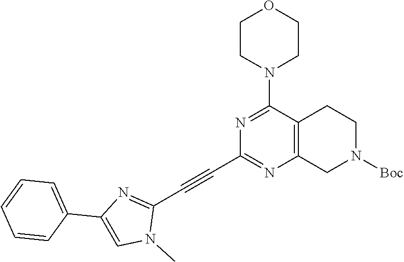 [SMILES: Cn1cc(-c2ccccc2)nc1C#Cc1nc2c(c(N3CCOCC3)n1)CCN(C(=O)OC(C)(C)C)C2]

### tert-Butyl 2-[2-(1-methyl-4-phenyl-imidazol-2-yl)ethynyl]-4-morpholino-6,8-dihydro-5H-pyrido[3,4-d]pyrimidine-7-carboxylate

Under an atmosphere of N₂, a solution of intermediate 9 (455 mg, 1.32 mmol) in DMF (2.5 mL) was added dropwise to a solution of intermediate 38 (150 mg, 0.53 mmol), CuI (5.0 mg, 26 μmol), XantPhos-Pd-G2 (74.0 mg, 79 μmol), and Xanthphos (45.8 mg, 79 μmol) in TEA (2.0 mL) and DMF (7.5 mL). The resultant mixture was stirred for 2 h at 80° C. before diluting with water (5 mL) and extracted with EtOAc (3×10 mL). The combined organic layers were washed with brine (20 mL), dried over anhydrous Na₂SO₄, and concentrated in vacuo. The residue was purified by silica gel column chromatography to afford the title compound (202 mg, 76.6%) as a brown solid. LCMS (ES+): 501.25 [MH]⁺.

Intermediates 44-47 and 70-76 and examples 6 and 16-19 were prepared similarly to intermediate 43, via Sonogashira coupling using XantPhos-Pd-G2; see Table 8 below (which is split in two parts).

TABLE 8
Sonogashira Coupling with XantPhos-Pd-G2

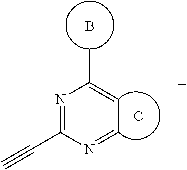

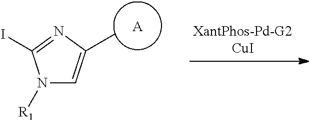

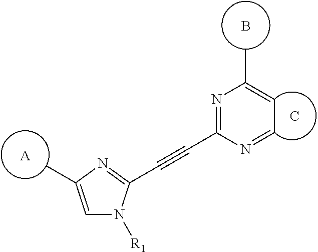

| | | | Intermediate, Form, |
|---|---|---|---|
| Int | Structure | Name | Yield, LCMS |
| 70 | 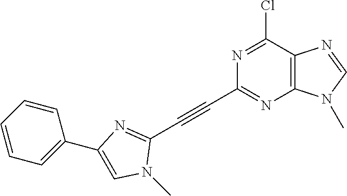 [SMILES: Cn1cc(-c2ccccc2)nc1C#Cc1nc(Cl)c2ncn(C)c2n1] | 6-Chloro-9-methyl-2-((1- methyl-4-phenyl-1H- imidazol-2-yl)ethynyl)-9H- purine | From intermediates 9 and 64 Yellow solid Yield 60 mg, 33.1% LCMS (ES+): 349.1 [MH]+. |
| 71 | 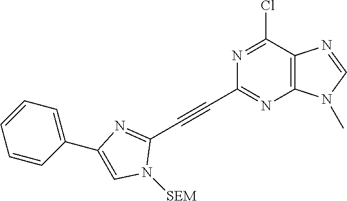 [SMILES: Cn1cnc2c(Cl)nc(C#Cc3nc(-c4ccccc4)cn3COCC[Si](C)(C)C)nc21] | 6-Chloro-9-methyl-2-((4- phenyl-1-((2- (trimethylsilyl) ethoxy)methyl)- 1H-imidazol-2- yl)ethynyl)-9H-purine | From intermediates 10 and 64 Brown solid Yield 240 mg, 49.7% LCMS (ES+): 465.3 [MH]+. |
| 72 | 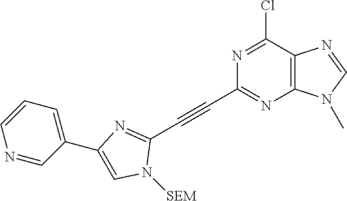 | 6-Chloro-9-methyl-2-((4- (pyridin-3-yl)-1-((2- (trimethylsilyl)ethoxy)meth yl)-1H-imidazol-2- yl)ethynyl)-9H-purine | From intermediates 49 and 64 Brown solid Yield 240 mg, 49.6% LCMS (ES+): 350.2 [MH]+. |
| 73 | 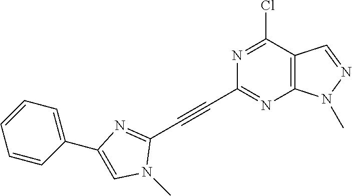 [SMILES: Cn1cc(-c2ccccc2)nc1C#Cc1nc(Cl)c2cnn(C)c2n1] | 4-Chloro-1-methyl-6-((1- methyl-4-phenyl-1H- imidazol-2-yl)ethynyl)-1H- pyrazolo[3,4-d]pyrimidine | From intermediates 9 and 65 Brown solid Yield 240 mg, 26.5% LCMS (ES+): 349.1 [MH]+. |
| 44 | 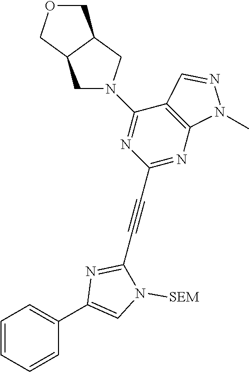 [SMILES: Cn1ncc2c(N3C[C@H]4COC[C@H]4C3)nc(C#Cc3nc(-c4ccccc4)cn3COCC[Si](C)(C)C)nc21] | 2-[2-[2-[4-[(3aS,6aR)- 1,3,3a,4,6,6a- hexahydrofuro[3,4-c]pyrrol- 5-yl]-1-methyl-pyrazolo[3,4- d]pyrimidin-6-yl]ethynyl]-4- phenyl-imidazol-1- yl]methoxy]ethyl-trimethyl- silane | From intermediates 10 and 36 Used without further purification |
| 74 | 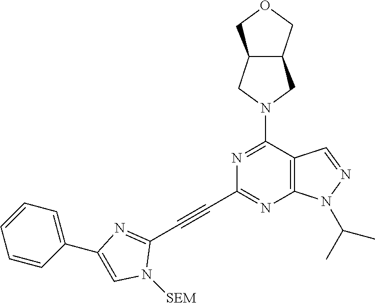 [SMILES: CC(C)n1ncc2c(N3C[C@H]4COC[C@H]4C3)nc(C#Cc3nc(-c4ccccc4)cn3COCC[Si](C)(C)C)nc21] | (3aR,6aS)-5-(1-isoPropyl-6- ((4-phenyl-1-((2- (trimethylsilyl)ethoxy)methyl)- 1H-imidazol-2- yl)ethynyl)-1H-pyrazolo[3,4- d]pyrimidin-4- yl)hexahydro-1H-furo[3,4- c]pyrrole | From intermediates 10 and 66 Yellow solid Yield 189 mg, 65.8% LCMS (ES+): 570.3 [MH]+. |
| 75 | 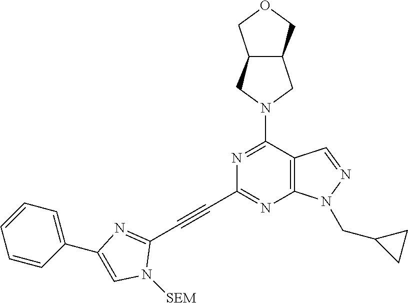 [SMILES: C[Si](C)(C)CCOCn1cc(-c2ccccc2)nc1C#Cc1nc(N2C[C@H]3COC[C@H]3C2)c2cnn(CC3CC3)c2n1] | (3aR,6aS)-5-(1- (Cyclopropylmethyl)-6-((4- phenyl-1-((2- (trimethylsilyl)ethoxy)methyl)- 1H-imidazol-2- yl)ethynyl)-1H-pyrazolo[3,4- d]pyrimidin-4- yl)hexahydro-1H-furo[3,4- c]pyrrole | From intermediates 10 and 67 Yellow solid Yield 120 mg, 63.8% LCMS (ES+): 582.2 [MH]+. |
| 76 | 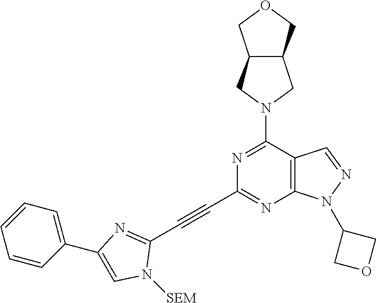 [SMILES: C[Si](C)(C)CCOCn1cc(-c2ccccc2)nc1C#Cc1nc(N2C[C@H]3COC[C@H]3C2)c2cnn(C3COC3)c2n1] | (3aR,6aS)-5-(1-(Oxetan-3- yl)-6-((4-phenyl-1-((2- (trimethylsilyl)ethoxy)methyl)- 1H-imidazol-2- yl)ethynyl)-1H-pyrazolo[3,4- d]pyrimidin-4- yl)hexahydro-1H-furo[3,4- c]pyrrole | From intermediates 10 and 69 Yellow solid Yield 100 mg, 35.6% LCMS (ES+): 584.3 [MH]+. |
| 45 | 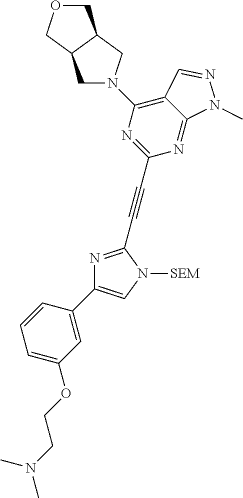 | 2-[3-[2-[2-[4-[(3aS,6aR)- 1,3,3a,4,6,6a- Hexahydrofuro[3,4- c]pyrrol-5-yl]-1-methyl- pyrazolo[3,4-d]pyrimidin-6- yllethynyl]-1-(2- trimethylsilylethoxymethyl) imidazol-4-yl]phenoxy]- N,N-dimethyl-ethanamine | From intermediates 14 and 36 Yellow solid Yield 130 mg, 50.0% LCMS (ES+): 629.4 [MH]+. |
| 46 | 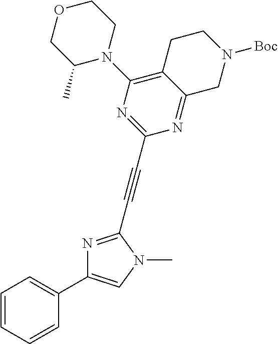 [SMILES: C[C@@H]1COCCN1c1nc(C#Cc2nc(-c3ccccc3)cn2C)nc2c1CCN(C(=O)OC(C)(C)C)C2] | tert-Butyl 4-[(3R)-3- methylmorpholin-4-yl]-2-[2- (1-methyl-4-phenyl- imidazol-2-yl)ethynyl]-6,8- dihydro-5H-pyrido[3,4- d]pyrimidine-7-carboxylate | From intermediates 9 and 39 Beige solid Yield 150 mg, 58.5% LCMS (ES+): 515.3 [MH]+. |
| 47 | 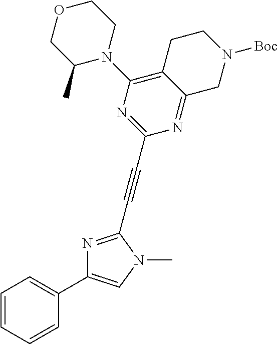 [SMILES: C[C@H]1COCCN1c1nc(C#Cc2nc(-c3ccccc3)cn2C)nc2c1CCN(C(=O)OC(C)(C)C)C2] | tert-Butyl 4-[(3S)-3- methylmorpholin-4-yl]-2-[2- (1-methyl-4-phenyl- imidazol-2-yl)ethynyl]-6,8- dihydro-5H-pyrido[3,4- d]pyrimidine-7-carboxylate | From intermediates 9 and 40 Beige solid Yield 200 mg, 49.8% LCMS (ES+): 515.3 [MH]+. |
| 16 | 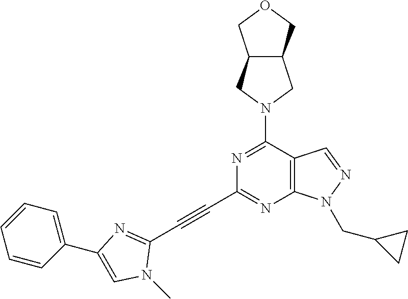 [SMILES: Cn1cc(-c2ccccc2)nc1C#Cc1nc(N2C[C@H]3COC[C@H]3C2)c2cnn(CC3CC3)c2n1] | (3aR,6aS)-5-(1- (Cyclopropylmethyl)-6-((1- methyl-4-phenyl-1H- imidazol-2-yl)ethynyl)-1H- pyrazolo[3,4-d]pyrimidin-4- yl)hexahydro-1H-furo[3,4- c]pyrrole | From intermediates 9 and 67 Yellow solid Yield 70.4 mg, 46.6% UPLC (Method C): Rt 1.61 min. HRMS (ES+/QToF) m/z: [M + H]⁺ Calcd for C₂₇H₂₈N₇O 466.2355; Found 466.2357. |
| 17 | 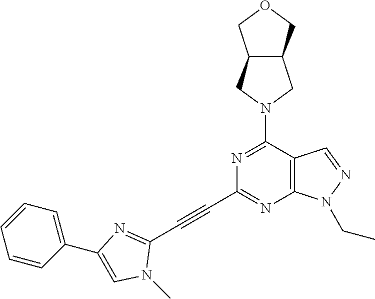 [SMILES: CCn1ncc2c(N3C[C@H]4COC[C@H]4C3)nc(C#Cc3nc(-c4ccccc4)cn3C)nc21] | (3aR,6aS)-5-(1-Ethyl-6-((1- methyl-4-phenyl-1H- imidazol-2-yl)ethynyl)-1H- pyrazolo[3,4-d]pyrimidin-4- yl)hexahydro-1H-furo[3,4- c]pyrrole | From intermediates 9 and 68 White solid Yield 84.6 mg, 54.4% UPLC (Method G): Rt 1.11 min. HRMS (ES+/QToF) m/z: [M + H]⁺ Calcd for C₂₅H₂₆N₇O 440.2199; Found 440.2198. |
| 18 | 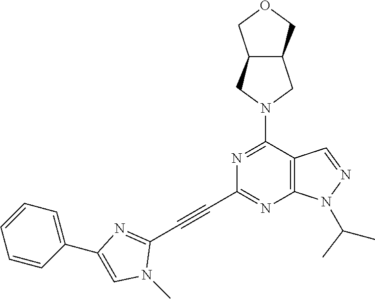 [SMILES: CC(C)n1ncc2c(N3C[C@H]4COC[C@H]4C3)nc(C#Cc3nc(-c4ccccc4)cn3C)nc21] | (3aR,6aS)-5-(1-isoPropyl-6- ((1-methyl-4-phenyl-1H- imidazol-2-yl)ethynyl)-1H- pyrazolo[3,4-d]pyrimidin-4- yl)hexahydro-1H-furo[3,4- c]pyrrole | From intermediates 9 and 66 White solid Yield 57.3 mg, 37.4% UPLC (Method C): Rt 1.58 min. HRMS (ES+/QToF) m/z: [M + H]⁺ Calcd for C₂₆H₂₈N₇O 454.2355; Found 454.2352. |
| 19 | 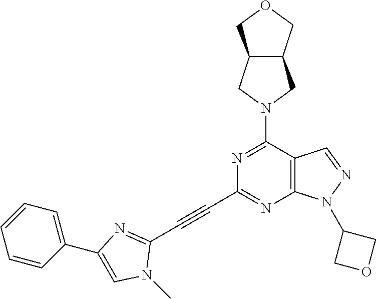 [SMILES: Cn1cc(-c2ccccc2)nc1C#Cc1nc(N2C[C@H]3COC[C@H]3C2)c2cnn(C3COC3)c2n1] | (3aR,6aS)-5-(6-((1-Methyl-4- phenyl-1H-imidazol-2- yl)ethynyl)-1-(oxetan-3-yl)- 1H-pyrazolo[3,4- d]pyrimidin-4- yl)hexahydro-1H-furo[3,4- c]pyrrole | From intermediates 9 and 69 White solid Yield 10.0 mg, 6.57% UPLC (Method C): Rt 1.61 min. LCMS (ES+): 468.2 [MH]+. HRMS (ES+/QToF) m/z: [M + H]⁺ Calcd for C₂₆H₂₆N₇O₂ 468.2148; Found 468.2154. |
| 6 | 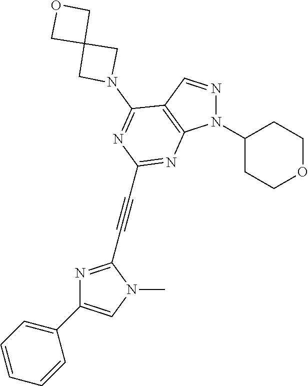 [SMILES: Cn1cc(-c2ccccc2)nc1C#Cc1nc(N2CC3(COC3)C2)c2cnn(C3CCOCC3)c2n1] | 6-[6-[2-(1-Methyl-4-phenyl- imidazol-2-yl)ethynyl]-1- tetrahydropyran-4-yl- pyrazolo[3,4-d]pyrimidin-4- yl]-2-oxa-6- azaspiro[3.3]heptane | From intermediates 9 and 28 White solid Yield 21.8 mg, 36.8% UPLC (Method A): Rt 2.12 min. HRMS (ES+/QToF) m/z: [M + H]⁺ Calcd for C₂₇H₂₈N₇O₂ 482.2304; Found 482.2303. |

### Example 20

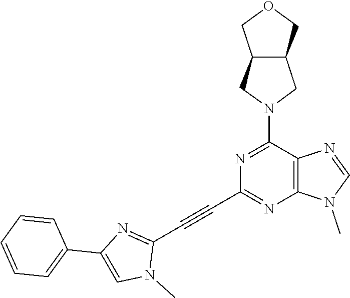 [SMILES: Cn1cc(-c2ccccc2)nc1C#Cc1nc(N2C[C@H]3COC[C@H]3C2)c2ncn(C)c2n1]

### (3aR,6aS)-5-(9-Methyl-2-((1-methyl-4-phenyl-1H-imidazol-2-yl)ethynyl)-9H-purin-6-yl)hexahydro-1H-furo[3,4-c]pyrrole

A solution of intermediate 70 (60 mg, 0.17 mmol), (3aR,6aS)-hexahydro-1H-furo[3,4-c]pyrrole (39 mg, 0.34 mmol), and TEA (76 μL, 0.51 mmol) in DMF (1.0 mL) was stirred for 1 h before quenching with water (5 mL) and extracted with EtOAc (2×10 mL). The combined organic layers were washed with sat aq NH₄Cl (3×5 mL), dried over anhydrous Na₂SO₄, and concentrated in vacuo. The residue was purified by reverse phase HPLC (NH₄HCO₃ buffered) to afford the title compound (19.9 mg, 26.8%) as a yellow solid. UPLC (Method C): Rt 1.39 min. HRMS (ES+/QToF) m/z: [M+H]⁺ Calcd for C₂₄H₂₄N₇O 426.2042; Found 426.2048.

Intermediates 77-82 and examples 21-24 were prepared similarly to example 20, by late stage SnAr of a halogenated pyrimidines; see Table 15 below.

TABLE 15
Late Stage SnAr of halogenated pyrimidines
SCHEME

| | | | Intermediate, Form, |
|---|---|---|---|
| Int | Structure | Name | Yield, LCMS |
| 77 | 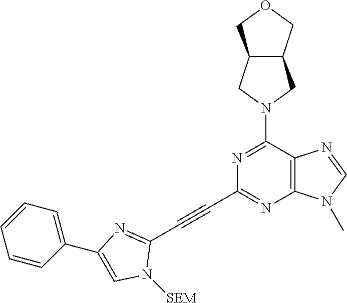 [SMILES: Cn1cnc2c(N3C[C@H]4COC[C@H]4C3)nc(C#Cc3nc(-c4ccccc4)cn3COCC[Si](C)(C)C)nc21] | (3aR,6aS)-5-(9-Methyl-2-((4- phenyl-1-((2- (trimethylsilyl)ethoxy)methyl)- 1H-imidazol-2- yl)ethynyl)-9H-purin-6- yl)hexahydro-1H-furo[3,4- c]pyrrole | From intermediate 71 and (3aR,6aS)- hexahydro-1H-furo[3,4- c]pyrrole Used without purification |
| 78 | 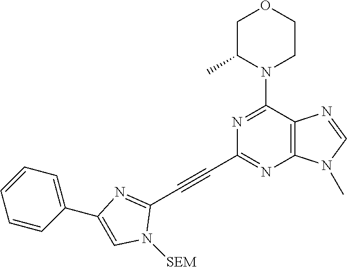 [SMILES: C[C@@H]1COCCN1c1nc(C#Cc2nc(-c3ccccc3)cn2COCC[Si](C)(C)C)nc2c1ncn2C] | (R)-3-Methyl-4-(9-methyl-2- ((4-phenyl-1-((2- (trimethylsilyl)ethoxy)methyl)- 1H-imidazol-2- yl)ethynyl)-9H-purin-6- yl)morpholine | From intermediate 71 and (3R)-3- methylmorpholine Used without purification |
| 79 | 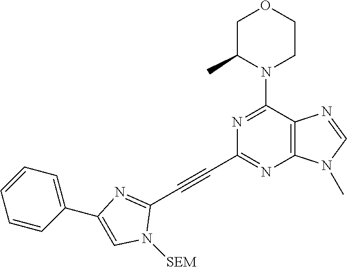 [SMILES: C[C@H]1COCCN1c1nc(C#Cc2nc(-c3ccccc3)cn2COCC[Si](C)(C)C)nc2c1ncn2C] | (S)-3-Methyl-4-(9-methyl-2- ((4-phenyl-1-((2- (trimethylsilyl)ethoxy)methyl)- 1H-imidazol-2- yl)ethynyl)-9H-purin-6- yl)morpholine | From intermediate 71 and (3S)-3- methylmorpholine Used without purification |
| 80 | 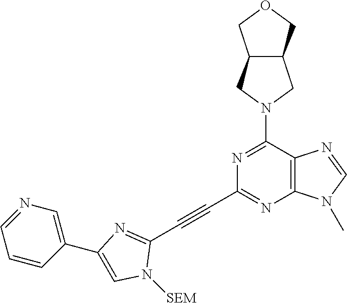 [SMILES: Cn1cnc2c(N3C[C@H]4COC[C@H]4C3)nc(C#Cc3nc(-c4cccnc4)cn3COCC[Si](C)(C)C)nc21] | (3aR,6aS)-5-(9-Methyl-2-((4- (pyridin-3-yl)-1-((2- (trimethylsilyl)ethoxy)meth yl)-1H-imidazol-2- yl)ethynyl)-9H-purin-6- yl)hexahydro-1H-furo[3,4- c]pyrrole | From intermediate 72 and (3aR,6aS)- hexahydro-1H-furo[3,4- c]pyrrole Used without purification |
| 81 | 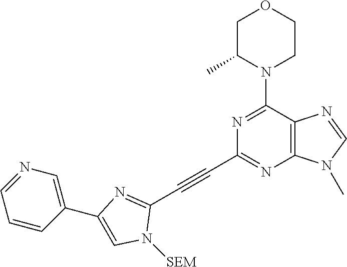 [SMILES: C[C@@H]1COCCN1c1nc(C#Cc2nc(-c3cccnc3)cn2COCC[Si](C)(C)C)nc2c1ncn2C] | (R)-3-Methyl-4-(9-methyl-2- ((4-(pyridin-3-yl)-1-((2- (trimethylsilyl)ethoxy)meth yl)-1H-imidazol-2- yl)ethynyl)-9H-purin-6- yl)morpholine | From intermediate 72 and (3R)-3- methylmorpholine Used without purification |
| 82 | 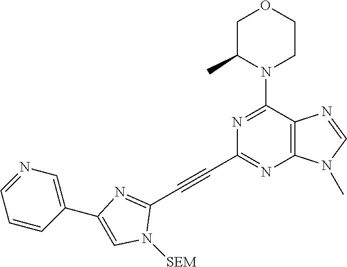 [SMILES: C[C@H]1COCCN1c1nc(C#Cc2nc(-c3cccnc3)cn2COCC[Si](C)(C)C)nc2c1ncn2C] | (S)-3-Methyl-4-(9-methyl-2- ((4-(pyridin-3-yl)-1-((2- (trimethylsilyl)ethoxy)methyl)- 1H-imidazol-2- yl)ethynyl)-9H-purin-6- yl)morpholine | From intermediate 72 and (3S)-3- methylmorpholine Used without purification |
| 21 | 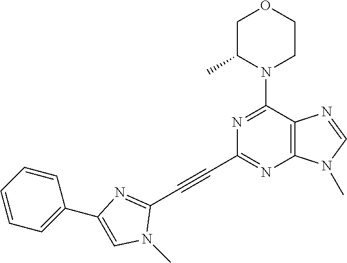 [SMILES: C[C@@H]1COCCN1c1nc(C#Cc2nc(-c3ccccc3)cn2C)nc2c1ncn2C] | (R)-3-Methyl-4-(9-methyl-2- ((1-methyl-4-phenyl-1H- imidazol-2-yl)ethynyl)-9H- purin-6-yl)morpholine | From intermediate 71 and (3R)-3- methylmorpholine Yellow solid Yield 13.1 mg, 16.9% UPLC (Method G): Rt 1.39 min. HRMS (ES+/QToF) m/z: [M + H]⁺ Calcd for C₂₃H₂₄N₇O 414.2042; Found 414.2042. |
| 22 | 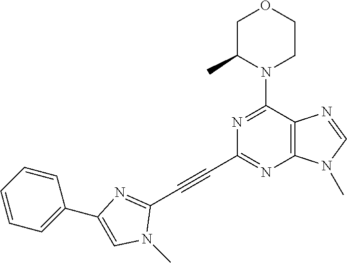 [SMILES: C[C@H]1COCCN1c1nc(C#Cc2nc(-c3ccccc3)cn2C)nc2c1ncn2C] | (S)-3-Methyl-4-(9-methyl-2- ((1-methyl-4-phenyl-1H- imidazol-2-yl)ethynyl)-9H- purin-6-yl)morpholine | From intermediate 71 and (3S)-3- methylmorpholine Yellow solid Yield 15.1 mg, 20.9% UPLC (Method G): Rt 1.40 min. HRMS (ES+/QToF) m/z: [M + H]⁺ Calcd for C₂₃H₂₄N₇O 414.2042; Found 414.2042. |
| 23 | 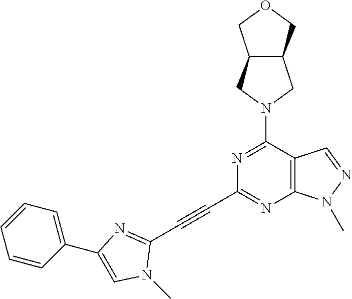 [SMILES: Cn1cc(-c2ccccc2)nc1C#Cc1nc(N2C[C@H]3COC[C@H]3C2)c2cnn(C)c2n1] | (3aR,6aS)-5-(1-Methyl-6-((1- methyl-4-phenyl-1H- imidazol-2-yl)ethynyl)-1H- pyrazolo[3,4-d]pyrimidin-4- yl)hexahydro-1H-furo[3,4- c]pyrrole | From intermediate 73 and (3aR,6aS)- hexahydro-1H-furo[3,4- c]pyrrole Yellow solid Yield 17.1 mg, 23.1% UPLC (Method C): Rt 0.83 min. HRMS (ES+/QToF) m/z: [M + H]⁺ Calcd for C₂₄H₂₄N₇O 426.2042; Found 426.2044. |
| 24 | 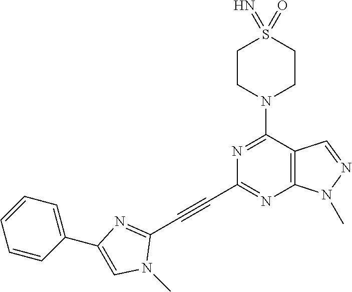 [SMILES: Cn1cc(-c2ccccc2)nc1C#Cc1nc(N2CCS(=N)(=O)CC2)c2cnn(C)c2n1] | 1-Imino-4-(1-methyl-6-((1- methyl-4-phenyl-1H- imidazol-2-yl)ethynyl)-1H- pyrazolo[3,4-d]pyrimidin-4- yl)-1l6-thiomorpholine 1- oxide | From intermediate 73 and 1-imino-1,4- thiazinane 1-oxide Yellow solid Yield 30.4 mg, 39.3% UPLC (Method C): Rt 1.143 min. HRMS (ES+/QToF) m/z: [M + H]⁺ Calcd for C₂₂H₂₃N₉OS 447.1715; Found 447.1714. |

### Example 7

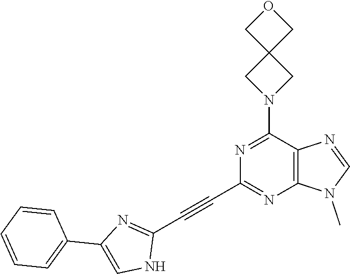 [SMILES: Cn1cnc2c(N3CC4(COC4)C3)nc(C#Cc3nc(-c4ccccc4)c[nH]3)nc21]

### 6-[9-Methyl-2-[2-(4-phenyl-1H-imidazol-2-yl)ethynyl]purin-6-yl]-2-oxa-6-azaspiro[3.3]heptane

TBAF (5.00 mL, 19.1 mmol) was added to a solution of intermediate 41 (45 mg, 85 μmol) in THF (5.0 mL), and the reaction was stirred for 16 h. The reaction was quenched with the addition of water (5 mL) and extracted with DCM (5×5 mL). The combined organic layers were washed with sat aq NH₄Cl (3×5 mL), dried over anhydrous Na₂SO₄, and concentrated in vacuo. The residue was purified by reverse phase HPLC (NH₄HCO₃ buffered) to afford the title compound (8.3 mg, 24.5%) as a white solid. UPLC (Method A): Rt 1.81 min. HRMS (ES+/QToF) m/z: [M+H]⁺ Calcd for C₂₂H₂₀N₇O 398.1729; Found 398.1727.

### Example 8

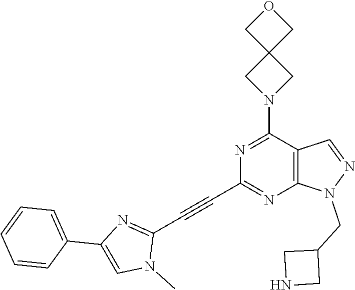 [SMILES: Cn1cc(-c2ccccc2)nc1C#Cc1nc(N2CC3(COC3)C2)c2cnn(CC3CNC3)c2n1]

### 6-[1-(Azetidin-3-ylmethyl)-6-[2-(1-methyl-4-phenyl-imidazol-2-yl)ethynyl]pyrazolo[3,4-d]pyrimidin-4-yl]-2-oxa-6-azaspiro[3.3]heptane

TFA (2.0 mc was added to a solution of intermediate 42 (80.0 mg, 141 μmol) in DCM (4.0 mL). The reaction was stirred for 1 h before concentration in vacuo and purification by reverse phase HPLC (NH—HCO₃ buffered) to afford the title compound (40.6 mg, 59.9%) as a yellow solid. UPLC (Method C): Rt 0.99 mD. LCMS (ES+): 467.3 [MH]⁺.

Examples 9-13 and 25-33 were prepared similarly to example 8, via TFA mediated deprotection; see Table 9 below.

TABLE 9
SEM or Boc Deprotection with TFA

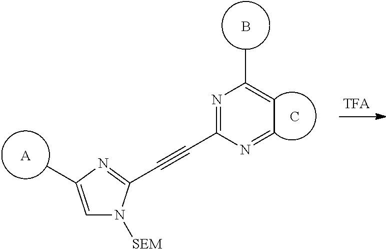

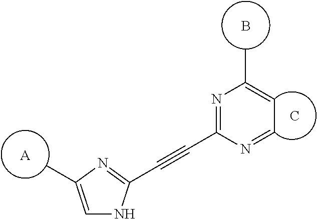

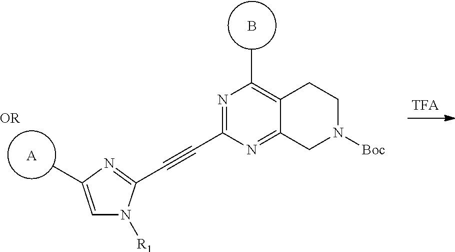

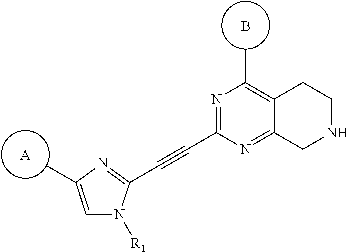

| | | | Intermediate, Form, |
|---|---|---|---|
| Ex | Structure | Name | Yield, LCMS |
| 9 | 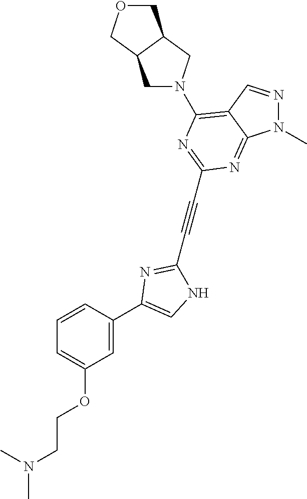 | (3aS,6aR)-5-[1-methyl-6-[2- (4-phenyl-1H-imidazol-2- yl)ethynyl]pyrazolo[3,4- d]pyrimidin-4-yl]- 1,3,3a,4,6,6a- hexahydrofuro[3,4- c]pyrrole | From intermediate 44 Yellow solid Yield 29.7 mg, 38.3% UPLC (Method D): Rt 1.68 min. HRMS (ES+/QToF) m/z: [M + H]⁺ Calcd for C₂₃H₂₂N₇O 412.1886; Found 412.1885. |
| 10 | 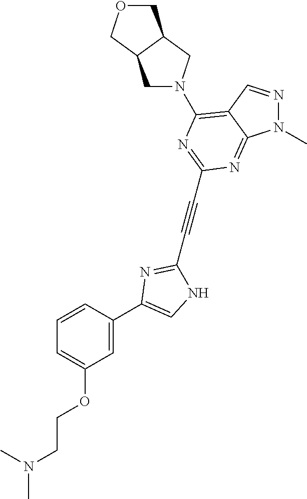 | 2-[3-[2-[2-[4-[(3aS,6aR)- 1,3,3a,4,6,6a- Hexahydrofuro[3,4- c]pyrrol-5-yl]-1-methyl- pyrazolo[3,4-d]pyrimidin-6- yl]ethynyl]-1H-imidazol-4- yl]phenoxy]-N,N-dimethyl- ethanamine | From intermediate 45 White solid Yield 22.0 mg, 24.1% UPLC (Method E): Rt 1.20 min. HRMS (ES+/QToF) m/z: [M+H]+ Calcd for C27H31N8O2 499.2570; Found 499.2567. |
| 25 | 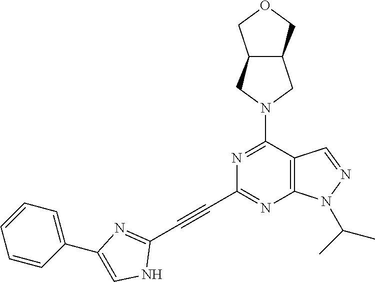 [SMILES: CC(C)n1ncc2c(N3C[C@H]4COC[C@H]4C3)nc(C#Cc3nc(-c4ccccc4)c[nH]3)nc21] | (3aR,6aS)-5-(1-isopPropyl- 6-((4-phenyl-1H-imidazol-2- yl)ethynyl)-1H-pyrazolo[3,4- d]pyrimidin-4- yl)hexahydro-1H-furo[3,4- c]pyrrole | From intermediate 74 White solid Yield 69.9 mg, 47.9% UPLC (Method E): Rt 1.08 min. HRMS (ES+/QToF) m/z: [M + H]+ Calcd for C₂₅H₂₅N₇O 440.2199; Found 440.2201. |
| 26 | 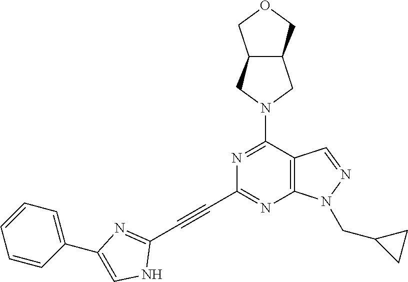 [SMILES: C(#Cc1nc(-c2ccccc2)c[nH]1)c1nc(N2C[C@H]3COC[C@H]3C2)c2cnn(CC3CC3)c2n1] | (3aR,6aS)-5-(1- (Cyclopropylmethyl)-6-((4- phenyl-1H-imidazol-2- yl)ethynyl)-1H-pyrazolo[3,4- d]pyrimidin-4- yl)hexahydro-1H-furo[3,4- c]pyrrole | From intermediate 75 Yellow solid Yield 33.5 mg, 43.0% UPLC (Method G): Rt 1.08 min. HRMS (ES+/QToF) m/z: [M + H]⁺ Calcd for C₂₆H₂₆N₇O 452.2199; Found 452.2200. |
| 27 | 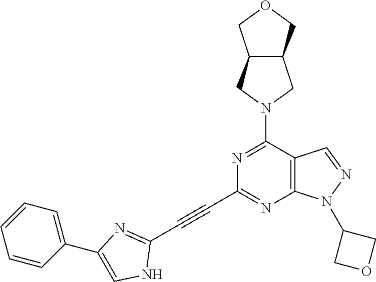 [SMILES: C(#Cc1nc(-c2ccccc2)c[nH]1)c1nc(N2C[C@H]3COC[C@H]3C2)c2cnn(C3COC3)c2n1] | (3aR,6aS)-5-(1-(Oxetan-3- yl)-6-((4-phenyl-1H- imidazol-2-yl)ethynyl)-1H- pyrazolo[3,4-d]pyrimidin-4- yl)hexahydro-1H-furo[3,4- c]pyrrole | From intermediate 76 Yellow solid Yield 17.6 mg, 20.5% UPLC (Method C): Rt 1.37 min. HRMS (ES+/QToF) m/z: [M + H]⁺ Calcd for C₂₅H₂₄N₇O₂ 454.1991; Found 454.1989. |
| 28 | 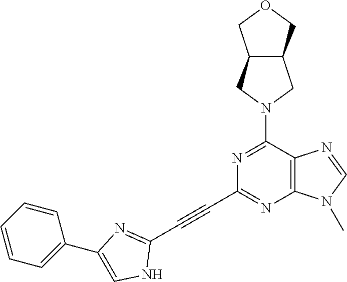 [SMILES: Cn1cnc2c(N3C[C@H]4COC[C@H]4C3)nc(C#Cc3nc(-c4ccccc4)c[nH]3)nc21] | (3aR,6aS)-5-(9-Methyl-2-((4- phenyl-1H-imidazol-2- yl)ethynyl)-9H-purin-6- yl)hexahydro-1H-furo[3,4- c]pyrrole | From intermediate 77 Yellow solid Yield 25.9 mg, 68.1% UPLC (Method E): Rt 1.44 min. HRMS (ES+/QToF) m/z: [M + H]⁺ Calcd for C₂₃H₂₂N₇O 412.1886; Found 412.1882. |
| 29 | 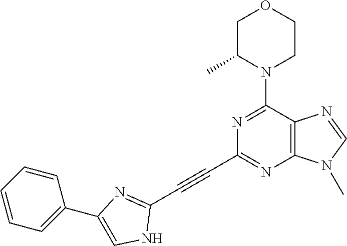 [SMILES: C[C@@H]1COCCN1c1nc(C#Cc2nc(-c3ccccc3)c[nH]2)nc2c1ncn2C] | (R)-3-Methyl-4-(9-methyl-2- ((4-phenyl-1H-imidazol-2- yl)ethynyl)-9H-purin-6- yl)morpholine | From intermediate 78 Yellow solid Yield 10.6 mg, 27.4% UPLC (Method E): Rt 0.98 min. HRMS (ES+/QToF) m/z: [M + H]⁺ Calcd for C₂₂H₂₂N₇O 400.1886; Found 400.1889. |
| 30 | 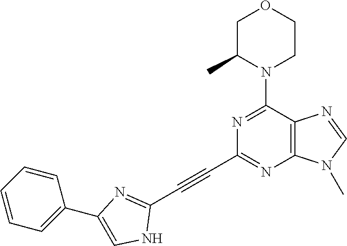 [SMILES: C[C@H]1COCCN1c1nc(C#Cc2nc(-c3ccccc3)c[nH]2)nc2c1ncn2C] | (S)-3-Methyl-4-(9-methyl-2- ((4-phenyl-1H-imidazol-2- yl)ethynyl)-9H-purin-6- yl)morpholine | From intermediate 79 Yellow solid Yield 19.9 mg, 43.8% UPLC (Method E): Rt 0.96 min. HRMS (ES+/QToF) m/z: [M + H]⁺ Calcd for C₂₂H₂₂N₇O 400.1886; Found 400.1888. |
| 31 | 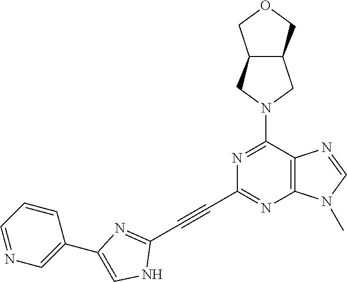 [SMILES: Cn1cnc2c(N3C[C@H]4COC[C@H]4C3)nc(C#Cc3nc(-c4cccnc4)c[nH]3)nc21] | (3aR,6aS)-5-(9-Methyl-2-((4- (pyridin-3-yl)-1H-imidazol- 2-yl)ethynyl)-9H-purin-6- yl)hexahydro-1H-furo[3,4- c]pyrrole | From intermediate 80 Yellow solid Yield 48.3 mg, 70.2% LCMS (ES+): 413.45 [MH]+. UPLC (Method E): Rt 0.67 min. HRMS (ES+/QToF) m/z: [M + H]⁺ Calcd for C₂₂H₂₁N₈O 413.1838; Found 413.1836. |
| 32 | 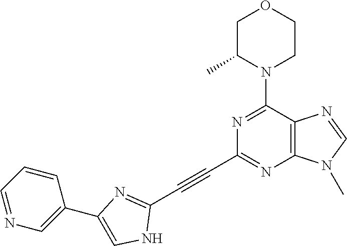 [SMILES: C[C@@H]1COCCN1c1nc(C#Cc2nc(-c3cccnc3)c[nH]2)nc2c1ncn2C] | (R)-3-Methyl-4-(9-methyl-2- ((4-(pyridin-3-yl)-1H- imidazol-2-yl)ethynyl)-9H- purin-6-yl)morpholine | From intermediate 81 Yellow solid Yield 20.6 mg, 30.9% UPLC (Method E): Rt 0.73 min. HRMS (ES+/QToF) m/z: [M + H]⁺ Calcd for C₂₁H₂₁N₈O 401.1838; Found 401.1839. |
| 33 | 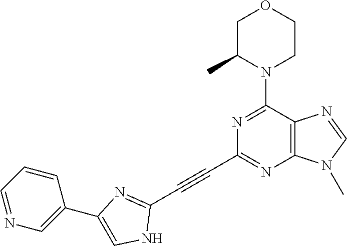 [SMILES: C[C@H]1COCCN1c1nc(C#Cc2nc(-c3cccnc3)c[nH]2)nc2c1ncn2C] | (S)-3-Methyl-4-(9-methyl-2- ((4-(pyridin-3-yl)-1H- imidazol-2-yl)ethynyl)-9H- purin-6-yl)morpholine | From intermediate 82 Yellow solid Yield 3.8 mg, 7.60% UPLC (Method E): Rt 0.74 min. HRMS (ES+/QToF) m/z: [M + H]⁺ Calcd for C₂₁H₂₁N₈O 401.1838; Found 401.1837. |
| 11 | 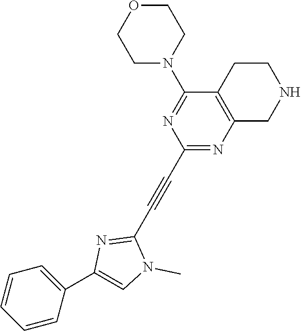 [SMILES: Cn1cc(-c2ccccc2)nc1C#Cc1nc2c(c(N3CCOCC3)n1)CCNC2] | 4-[2-[2-(1-Methyl-4-phenyl- imidazol-2-yl)ethynyl]- 5,6,7,8- tetrahydropyrido[3,4- d]pyrimidin-4- yl]morpholine | From intermediate 43 White solid Yield 41.5 mg, 51.5% UPLC (Method A): Rt 1.61 min. HRMS (ES+/QToF) m/z: [M + H]⁺ Calcd for C₂₃H₂₅N₆O 401.2090; Found 401.2087. |
| 12 | 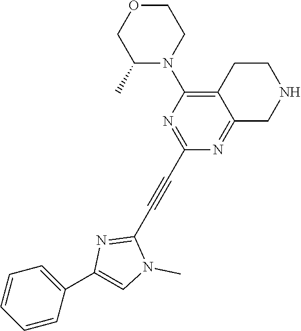 [SMILES: C[C@@H]1COCCN1c1nc(C#Cc2nc(-c3ccccc3)cn2C)nc2c1CCNC2] | (3R)-3-Methyl-4-[2-[2-(1- methyl-4-phenyl-imidazol- 2-yl)ethynyl]-5,6,7,8- tetrahydropyrido[3,4- d]pyrimidin-4- yl]morpholine | From intermediate 46 Yellow solid Yield 60.6 mg, 74.6% UPLC (Method C): Rt 1.04 min. HRMS (ES+/QToF) m/z: [M + H]⁺ Calcd for C₂₄H₂₇N₆O 415.2246; Found 415.2243. |
| 13 | 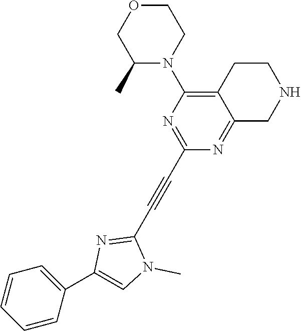 [SMILES: C[C@H]1COCCN1c1nc(C#Cc2nc(-c3ccccc3)cn2C)nc2c1CCNC2] | (3S)-3-Methyl-4-[2-[2-(1- methyl-4-phenyl-imidazol- 2-yl)ethynyl]-5,6,7,8- tetrahydropyrido[3,4- d]pyrimidin-4- yl]morpholine | From intermediate 47 Brown solid Yield 69.4 mg, 42.5% UPLC (Method C): Rt 1.10 min. HRMS (ES+/QToF) m/z: [M + H]⁺ Calcd for C₂₄H₂₇N₆O 415.2246; Found 415.2242. |

### Example 14

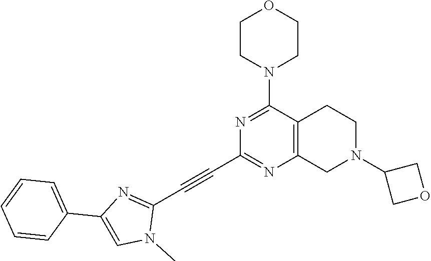 [SMILES: Cn1cc(-c2ccccc2)nc1C#Cc1nc2c(c(N3CCOCC3)n1)CCN(C1COC1)C2]

### 4-[2-[2-(1-Methyl-4-phenyl-imidazol-2-yl)ethynyl]-7-(oxetan-3-yl)-6,8-dihydro-5H-pyrido[3,4-d]pyrimidin-4-yl]morpholine

A solution of example 11 (120 mg, 300 μmol) and 3-oxetanone (176 μL, 3.00 mmol) in THF (5.0 mL) was stirred at 70° C. for 30 h before cooling to room temperature. Once cooled, NaBH₃CN (94.2 mg, 1.50 mmol) was added in several portions and the reaction was stirred for 16 h. The reaction was quenched with water (5 mL) and extracted with EtOAc (3×15 mL). The combined organic layers were concentrated in vacuo and the residue was purified by reverse phase HPLC (HCOOH buffered) to afford the title compound (26.4 mg, 19.0%) as a brown solid. UPLC (Method E): Rt 1.57 min. HRMS (ES+/QToF) m/z: [M+H]⁺ Calcd for C₂₆H₂₉N₆O₂ 457.2352; Found 457.2348.

### Example 15

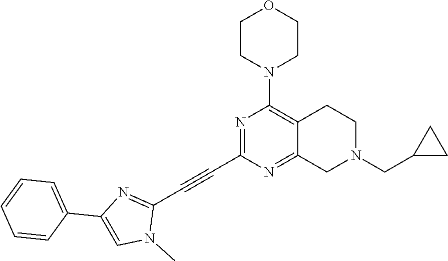 [SMILES: Cn1cc(-c2ccccc2)nc1C#Cc1nc2c(c(N3CCOCC3)n1)CCN(CC1CC1)C2]

### 4-[7-(Cyclopropylmethyl)-2-[2-(1-methyl-4-phenyl-imidazol-2-yl)ethynyl]-6,8-dihydro-5H-pyrido[3,4-d]pyrimidin-4-yl]morpholine

Under an atmosphere of N₂, a suspension of example 11 (140 mg, 350 μmol), K₂CO₃ (144 mg, 1.05 mmol), and (iodomethyl)cyclopropane (191 mg, 1.05 mmol) in DMF (10 mL) was stirred at 80° C. for 1 h. The reaction was then concentrated in vacuo and the residue purified by reverse phase HPLC (NH₄HCO₃ buffered) to afford the title compound (28.7 mg, 17.8%) as a yellow solid. UPLC (Method E): Rt 1.68 min. LCMS (ES+): 455.3 [MH]+.

### Biochemical Human PDE10A Activity Assay—PDE10A2 Phosphate Sensor Assay

Semi Log compound dilutions starting at a final concentration of 50 μM were dispensed into a black 384 well plate alongside, DMSO and an Inhibited control using the Echo Acoustic dispenser. Both Human PDE10A2 and CD73 in a tris-based assay buffer at the final assay concentrations of 0.25 nM and 1 nM respectively, were pre incubated with the compounds for 15 minutes at room temperature, prior to the addition of the substrates, cGMP, and Phosphate Sensor, also diluted in a tris-based assay buffer at the final assay concentrations of 3 μM and 0.9 μM, respectively. The plate was incubated for a further 35 minutes at room temperature before the fluorescence intensity was measured using an optical filter of Ex 430 nm/Em 450 nm on the BMG CLARIOStar Plate Reader. Data was analysed using a 4-parameter fit.

Cell Based Human PDE10A Activity Assay—cAMP HTRF Assay in HEK 293 rhPDE10A2 Cell Line

Semi log compound dilutions starting at a final concentration of 10 μM were dispensed into a white 384 well plate, alongside DMSO and an Inhibited control using the Tecan D300e digital dispenser. HEK 293 cells overexpressing recombinant human PDE10A2 were seeded on top of the compounds at 2500 cells/well in a volume 5 μL/well. The plate was incubated at room temperature for 60 minutes. To induce endogenous cAMP, 5 μL/well of Forskolin at a final assay concentration of 10 μM was added to the plate. The plate was incubated at room temperature for a further 45 minutes. cAMP HTRF detection reagents were added, and the plate was incubated for 60 minutes at room temperature. The FRET signal was measured using an HTRF optical filter (337/620/665) on the BMG PHERAstar FS Plate Reader. Data was analysed using a 4-parameter fit.

TABLE 10
PDE10 inhibition data for compounds of the invention

| | | cAMP HTRF Assay |
|---|---|---|
| | PDE10A2 Phosphate | in HEK 293 |
| | Sensor Enzyme Assay | rhPDE10A2 cell line |
| Example | (pIC₅₀) | (pIC₅₀) |
| 1 | 9.26 | 8.93 |
| 2 | 8.62 | 8.81 |
| 3 | 9.99 | 9.56 |
| 4 | 9.00 | 8.20 |
| 5 | 9.37 | 9.37 |
| 6 | 9.20 | 7.83 |
| 7 | 9.22 | 8.53 |
| 8 | 8.45 | 8.24 |
| 9 | 10.6 | 9.22 |
| 10 | 10.4 | 9.18 |
| 11 | 9.80 | 8.73 |
| 12 | 10.40 | 9.36 |
| 13 | 8.92 | <6 |
| 14 | 9.35 | 8.75 |
| 15 | 8.72 | 6.25 |
| 16 | 10.10 | 8.38 |
| 17 | 10.50 | 9.48 |
| 18 | 9.99 | 9.21 |
| 19 | 9.41 | 7.95 |
| 20 | 9.53 | 8.06 |
| 21 | 10.30 | 9.38 |
| 22 | 9.22 | ND |
| 23 | 10.40 | 9.61 |
| 24 | 10.10 | 8.92 |
| 25 | 9.95 | ND |
| 26 | 9.99 | ND |
| 27 | 9.49 | 7.49 |
| 28 | 9.34 | 7.47 |
| 29 | 9.92 | 8.46 |
| 30 | 8.97 | <6 |
| 31 | 8.95 | 8.40 |
| 32 | 9.90 | 8.63 |
| 33 | 8.68 | 7.76 |

The data in the table above show that the compounds of the invention, are potent PDE10A inhibitors, and may therefore be suitable for use in the treatment of inflammatory bowel diseases, such as ulcerative colitis and/or Crohn's disease.

### Determination of CNS Penetration In Vivo

Male Sprague Dawley Rats 300-350 g (Charles River, UK) were group housed, n=3, under a 12 hour light/dark cycle with food and water available ad libitum. Two days prior to dosing, animals were anaesthetised with inhaled isoflurane, and the right jugular vein was exposed and surgically cannulated. Animals were then housed singly for recovery, and throughout the remaining procedure. On the day of dosing animals were weighed, tail marked and dosed intravenously via the indwelling cannula with compound at 1 mg/kg in a volume of 3-5 mL/kg. Animals were culled at 10-30 min post dose via intravenous administration of pentobarbital. Post mortem blood was withdrawn via cardiac puncture, and briefly stored in K2 EDTA blood tubes on ice before being spun at 14,000 g for 4 min at 4° C. Plasma was withdrawn into a 96 well plate, placed on dry ice and stored at −80° C. Brains were quickly dissected and placed on dry ice before storage at −80° C.

Following dosing of test compound (intravenous) to Male Sprague-Dawley Rats, animals are sacrificed at one timepoint. Plasma is isolated from whole blood following cardiac exsanguination by centrifugal blood fractionation and whole brains isolated. Samples are stored on-ice and transferred to the Bioanalytical lab storage at −80° C. Bioanalysis of plasma and brain samples is performed as detailed below.

### Plasma Bioanalysis

Typically, a 1.00 mg/mL DMSO stock was used to prepare calibration standards of test compound in the range 1.00-6,000 ng/mL. Calibration lines were prepared by printing known masses of analyte into a 96-well plate in the range 25 to 150,000 pg. A volume of 25 μL of control male Sprague-Dawley Rat plasma was added to each well to prepare calibration standards at the appropriate concentration across the calibration range. Experimental samples were thawed to room temperature and 25 μL aliquots were added to the 96-well precipitation plate alongside the calibration lines. Samples were extracted using protein precipitation (agitation for at least 5 min at RT with 400 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex AP16500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value were included in the calibration line (±20% at the LLoQ) and any samples that fell outside of the limits of the calibration line were deemed to be less than or above the limit of quantification (LLoQ/ALoQ).

### Brain Bioanalysis

Typically, a 1.00 mg/mL DMSO stock was used to prepare calibration standards of test compound in the range 3.00-18,000 ng/mL. Calibration lines were prepared by printing known masses of analyte into a 96-well plate in the range 25 to 150,000 pg. A volume of 25 μL of control male Sprague-Dawley Rat brain homogenate (containing 8.33 mg of brain tissue) was added to each well to prepare calibration standards at the appropriate concentration across the calibration range.

To prepare control and experimental brain homogenates, brains were thawed at room temperature, weighed and a volume of diluent added (50:50 MeCN/H₂O) in the ratio of 2 mL per gram of brain. Homogenisation of brains was performed by bead-beater homogenisation using Precellys Evolution and CKMix50 7 mL mixed ceramic bead homogenisation tubes.

Aliquots of 25 μL experimental sample were extracted alongside the calibration lines using protein precipitation (agitation for at least 5 min at room temperature with 400 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex AP16500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value (±20% at the LLoQ) were included in the calibration line and any samples that fell outside of the limits of the calibration line were deemed to be less than or above the limit of quantification (LLoQ/ALoQ).

### Determination of Brain to Plasma Ratio and Free Brain Concentrations.

Total CNS penetrance was calculated by dividing the concentration in the brain by the concentration in plasma for each timepoint. The mean brain to plasma ratio (Br:PI) was calculated by averaging these ratios (defining which timepoints were used).

The free drug hypothesis states that only unbound compound is able to interact with and elicit a pharmacological effect. Therefore, it is desirable for compounds to have a high free brain concentration. To calculate the free concentrations in each matrix, the determined concentrations are multiplied by the % free value as determined by plasma protein binding and brain tissue binding studies using rapid equilibrium dialysis. These values are then converted to molar concentrations to give a nanomolar free result at each timepoint.

The Kpuu is calculated as the ratio of free drug fraction unbound in brain to free drug unbound in plasma.

TABLE 11
Brain to plasma partitioning (Kpuu)
for compounds of the invention

| | | | Dose | |
|---|---|---|---|---|
| Example | Formulation | Route | [mg/kg] | Kpuu |
| 7 | 10% NMP + 90% − 30% HPBC | IV | 1 | 0.02 |
| | in saline solution. | | | |
| 24 | 10% NMP + 90% − 30% HPBC | IV | 1 | 0.02 |
| | in saline solution. | | | |
| 32 | 10% NMP + 90% − 30% HPBC | IV | 0.25 | 0.10 |
| | in saline solution. | | | |
| H | 10% NMP + 90% − 30% HPBC | IV | 1 | 0.98 |
| | in saline solution. | | | |
| J | 10% NMP + 90% − 30% HPBC | IV | 1 | 1.36 |
| | in saline solution. | | | |

### Assessing PDE10A Inhibitors for Use in the Treatment of Ulcerative Colitis

To explore the role of PDE10A in ulcerative colitis (UC) the Genotype-Tissue Expression (GTEx) database was used to look at PDE10A RNA expression in normal and diseased tissues. Alongside this, expression levels of guanylate cyclase 2C (GUCY2C) were also assessed. GUCY2C is an enzyme which synthesises cGMP in response to the endogenous peptides guanylin and uroguanylin as well as E. coli heat-stable enterotoxin.

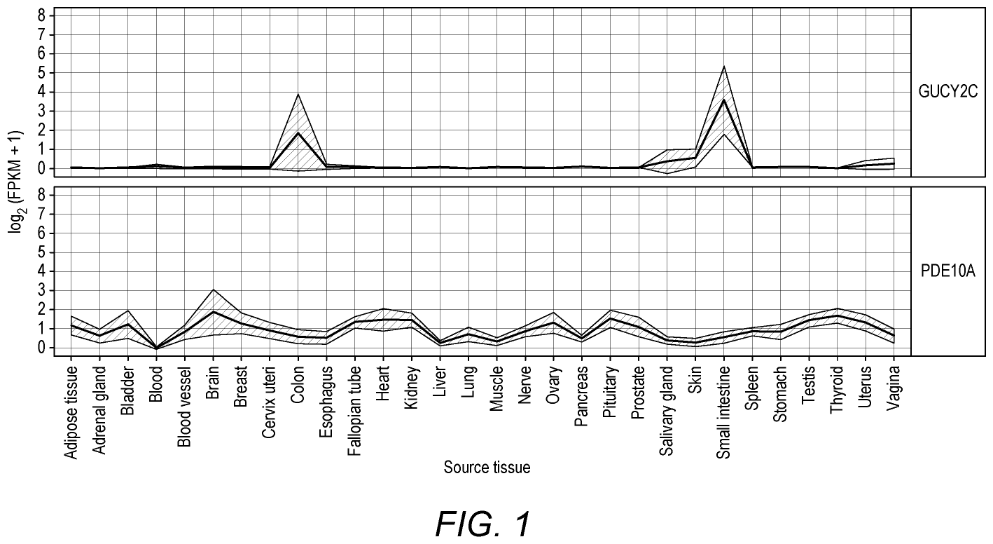

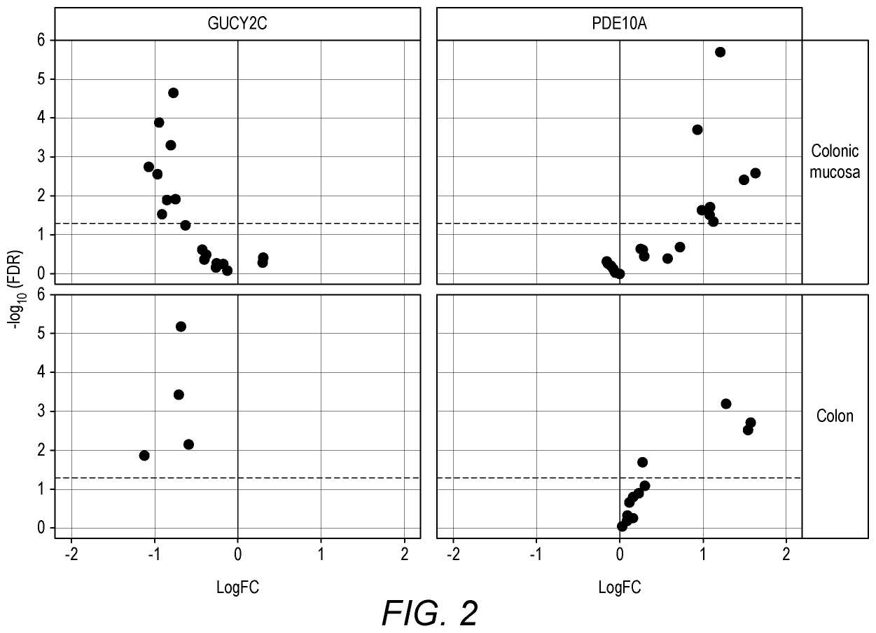

As previously described in the literature, in normal tissue PDE10A is expressed at low levels except in brain (as shown in FIG. 1). However in colonic mucosa and colon tissue from ulcerative colitis patients, PDE10A expression levels were significantly upregulated compared to normal controls (as shown in FIG. 2). This is a finding that has not been previously described in the literature and highlighting a potential undiscovered role for PDE10A in IBD pathology.

GUCY2C was seen to be specifically expressed at high levels in normal colon and small intestine (as shown in FIG. 1) suggesting a role for this enzyme in normal gut homeostasis. In UC colonic mucosa and colon, GUCY2C was significantly downregulated (as shown in FIG. 2), a finding that has previously been described in the literature.

Guanylate cyclase-C and cGMP signalling is downregulated in ulcerative colitis (Brenna et al. The guanylate cyclase-C signaling pathway is down-regulated in inflammatory bowel disease Scand J Gastroenterol. 50(10), 1241-52 (2015)) and decreases in expression of guanylate cyclase 2C, guanylin, and uroguanylin correlate with severity of disease. (Lan et al. Expression of guanylate cyclase-C, guanylin, and uroguanylin is downregulated proportionally to the ulcerative colitis disease activity index Sci Rep. 6, 25034, (2016) published online 29 Apr. 2016 doi: 10.1038/srep25034). This suggests that reduced cGMP signalling plays a role in UC pathology. cGMP in the GI tract has also been shown to play a role in fluid and electrolyte secretion, barrier function, inflammation and proliferation (Waldman et al. Guanylate cyclase-C as a therapeutic target in gastrointestinal disorders., Gut. 67(8), 1543-1552 (2018)).

While less studied in inflammation than cAMP, reduced cGMP signalling has also been shown to increase inflammation in other systems (Ahluwalia et al. Antiinflammatory activity of soluble guanylate cyclase: cGMP-dependent down-regulation of P-selectin expression and leukocyte recruitment. Proc Natl Acad Sci USA. 101(5), 1386-91 (2004); Raposo et al. Role of iNOS-NO-cGMP signaling in modulation of inflammatory and myelination processes. Brain Res Bull. 104, 60-73 (2014)).

Taken together, in UC colon and colonic mucosa, cGMP hydrolysing activity by PDE10A would be increased and cGMP synthesizing activity by guanylate cyclase 2C would be decreased resulting in a net decrease in cGMP levels and signalling. The therapeutic potential of inhibitors of PDE10A to treat inflammatory bowel diseases was assessed using tissue samples from inflamed colonic mucosa from ulcerative colitis patients.

The effect of selective PDE10A inhibition was tested on inflamed colonic mucosa from ulcerative colitis patients taken during routine endoscopy (Protocol 1 detailed below). These samples retain a disease phenotype in ex-vivo culture, secrete high basal levels of inflammatory cytokines, and represent a highly relevant and translational disease model. The effect of the PDE10A inhibitors on levels of the inflammatory cytokines IL-6 and IL-8 released from these tissue samples were measured. Both IL-6 and IL-8 are key regulators in ulcerative colitis pathology and their levels correlate with disease severity (Waldner M J et al. Master regulator of intestinal disease: IL-6 in chronic inflammation and cancer development. Semin Immunol. 26(1), 75-9 (2014); Bernardo D et al. IL-6 promotes immune responses in human ulcerative colitis and induces a skin-homing phenotype in the dendritic cells and T-cells they stimulate. Eur J Immunol. 42(5), 1337-53 (2012); Pearl D S, Cytokine mucosal expression in ulcerative colitis, the relationship between cytokine release and disease activity. J Crohns Colitis. 7(6), 481-9 (2013)).

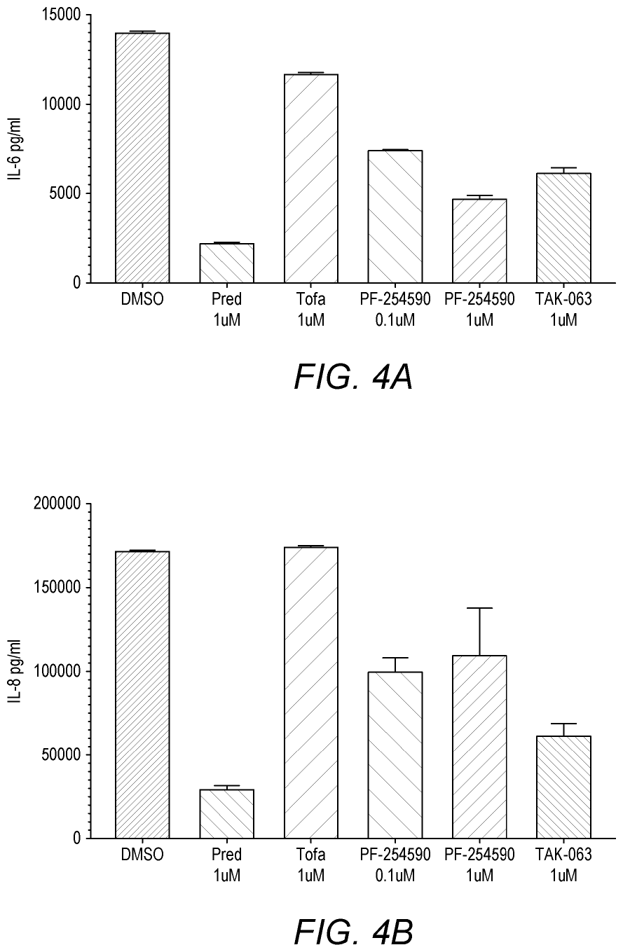

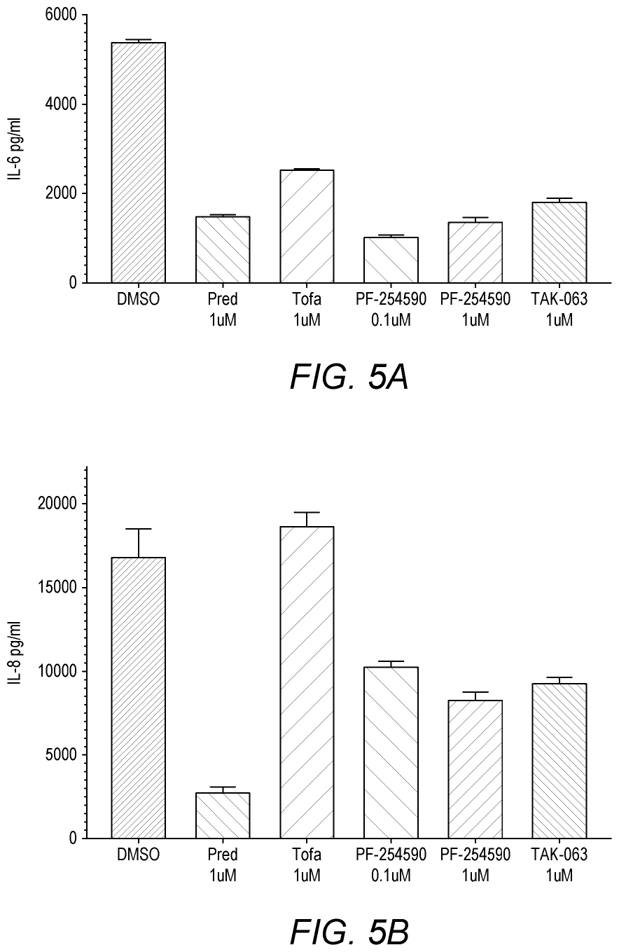

The structurally distinct PDE10A inhibitors PF-02545920 and TAK-063 were tested alongside two positive control compounds, the steroid prednisolone and the Janus kinase inhibitor tofacitinib, in colon biopsy samples from two ulcerative colitis patients. These colon biopsies retain an inflammatory phenotype and secrete high levels of inflammatory cytokines in ex-vivo culture. Selective PDE10A inhibition significantly reduced the secreted levels of IL-6 and IL-8 when compared to the DMSO vehicle (FIGS. 4 and 5). This reduction was comparable to that seen with the positive controls. PF-02545920 was tested at concentrations of 0.1 μM and 1 μM. TAK-063 was tested at a concentration of 1 uM. The doses tested of each inhibitor will result in selective PDE10A inhibition over the other PDE family members.

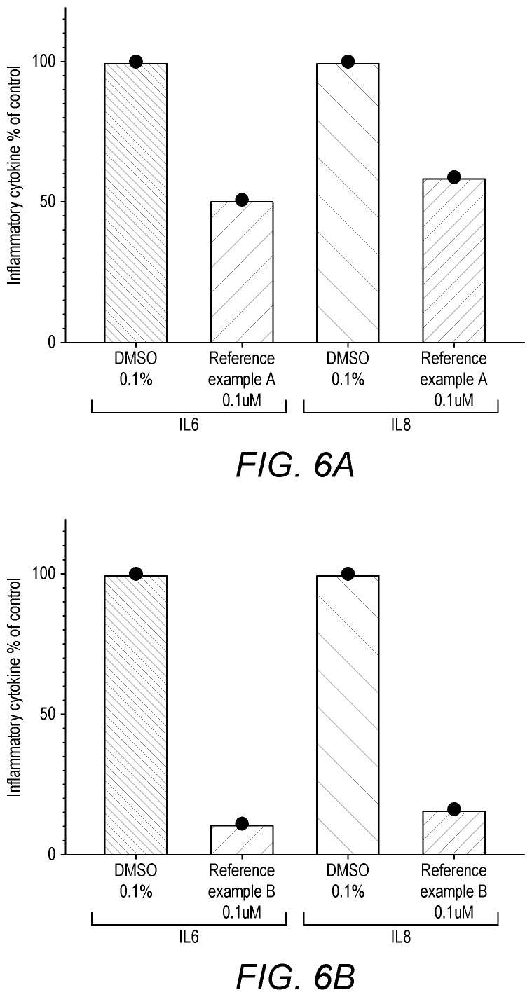

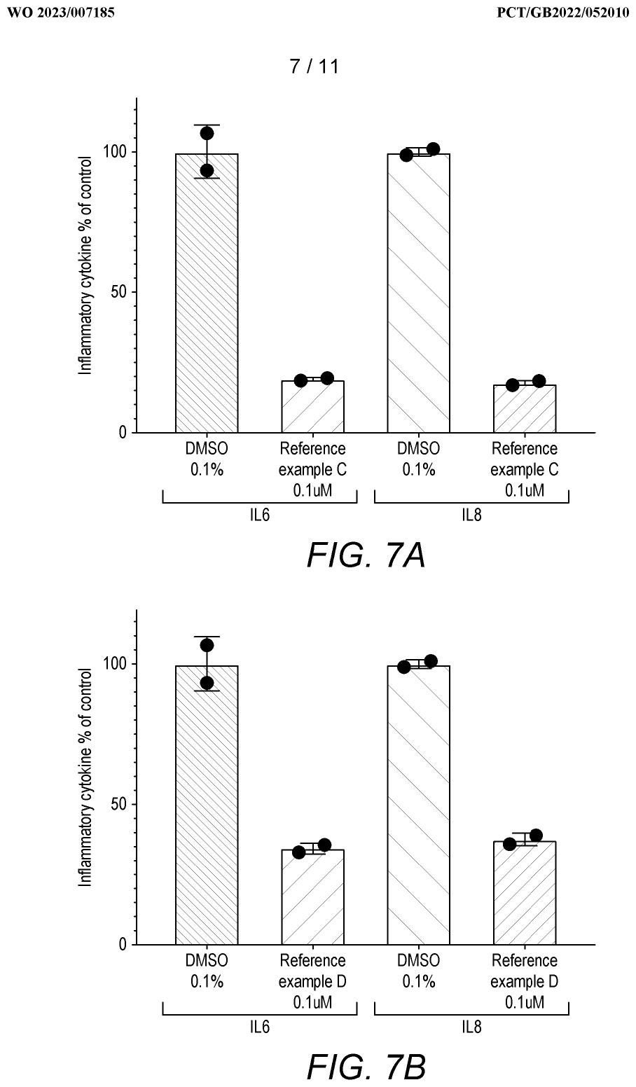

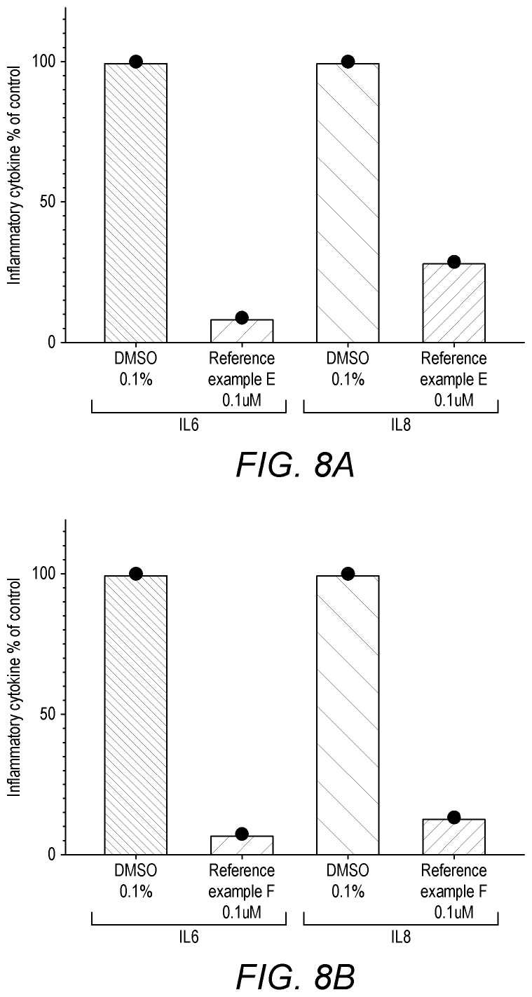

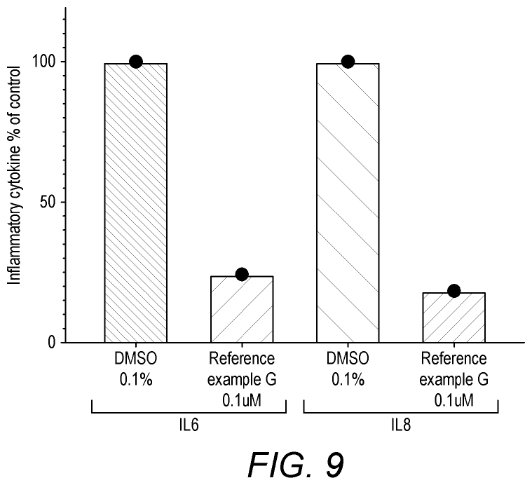

PDE10A inhibitors, Reference Examples A to G (the synthesis of which is outlined below together with their PDE10A inhibitory activity) were tested at a concentration of 100 nM (concentration selective for PDE10A inhibition), and were found to significantly reduce the secreted levels of IL-6 and IL-8 when compared to the vehicle control. The ability of selective PDE10A inhibition to significantly reduce levels of pathologic inflammatory cytokines in ex-vivo UC patient-derived colon tissue demonstrates the therapeutic utility of PDE10A inhibitors for the treatment of UC. The results are shown in FIGS. 6 to 9.

The structure of PF-02545920 is below. PF-02545920 is a potent and selective cyclic nucleotide PDE10A competitive inhibitor with a reported IC₅₀ value of 1.26 nM. PF-02545920 has been investigated in clinical trials for the treatment of Huntington's Disease. Patients were given 5 or 20 mg of PF-02545920 twice daily.

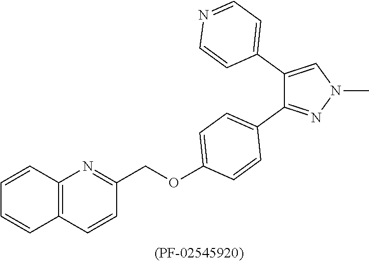 [SMILES: Cn1cc(-c2ccncc2)c(-c2ccc(OCc3ccc4ccccc4n3)cc2)n1]

In isolated enzyme biochemical assays, PF-02545920 has been shown to be a highly selective PDE10A inhibitor with an IC₅₀ for PDE10A<5 nM and IC₅₀s for other PDE family members >1 μM (Grauer S M et. al. Phosphodiesterase 10A inhibitor activity in preclinical models of the positive, cognitive, and negative symptoms of schizophrenia. J Pharmacol Exp Ther. 2009 331(2), 574-90). Therefore, at the test concentration of 0.1 μM and 1 μM in an ex-vivo tissue assay, PF-02545920 will selectively inhibit PDE10A.

The structure of TAK-063 below. TAK-063 was studied in a phase 2 clinical trial for the treatment of people with schizophrenia. TAK-063 was given at 20 mg once per day but may be reduced to 10 mg once per day if the higher dose was intolerable.

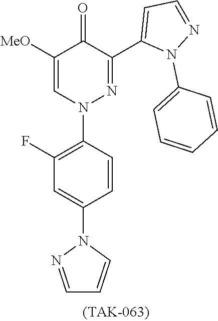 [SMILES: COc1cn(-c2ccc(-n3cccn3)cc2F)nc(-c2ccnn2-c2ccccc2)c1=O]

In isolated enzyme biochemical assays, TAK-063 has been shown to be a highly selective PDE10A inhibitor with an IC₅₀ for PDE10A of 0.3 nM and IC₅₀s for other PDE family members >5 μM (Kunitomo J et. al. Discovery of 1-[2-fluoro-4-(1H-pyrazol-1-yl)phenyl]-5-methoxy-3-(1-phenyl-1H-pyrazol-5-yl)pyridazin-4(1H)-one (TAK-063), a highly potent, selective, and orally active phosphodiesterase 10A (PDE10A) inhibitor. J Med. Chem. 57(22), 9627-43 (2014)) Therefore, at the test concentration of 1 uM in an ex-vivo tissue assay, TAK-063 will selectively inhibit PDE10A.

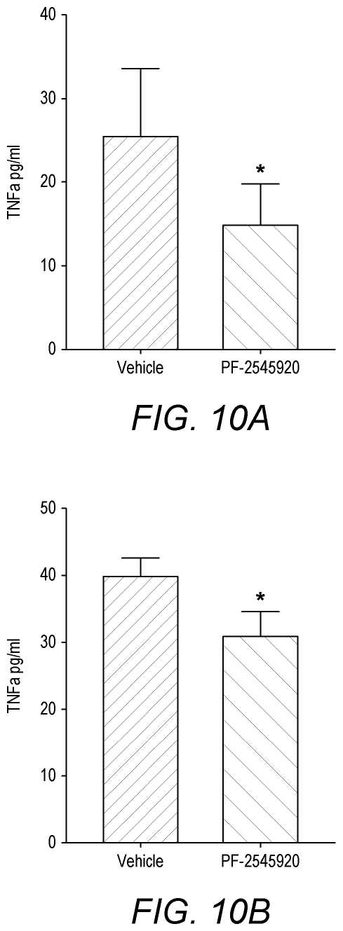

The effect of selective PDE10A inhibition was also tested on inflamed colonic mucosa from pharmacotherapy treatment-refractory ulcerative colitis patients taken during colon resection surgery (Protocol 2 detailed below). The PDE10A inhibitor PF-02545920 (1 μM) was tested in colon samples from two ulcerative colitis patients. The effect of selective PDE10A inhibition on levels of the inflammatory cytokine TNFα released from these tissue samples were measured. TNFα is a pro-inflammatory mediator that is expressed at high levels in the colonic mucosa of patients with UC and is the target of anti-TNFα biologics which have demonstrated efficacy in the treatment of UC (Pugliese D. et al. Anti TNF-α therapy for ulcerative colitis: current status and prospects for the future., Expert Rev Clin Immunol. 13(3), 223-233 (2017)). Selective PDE10A inhibition significantly reduced the secreted levels of TNFα compared to the DMSO vehicle (FIG. 10).

The ability of selective PDE10A inhibition to significantly reduce levels of inflammatory cytokines in UC patient-derived colonic mucosa demonstrates the therapeutic utility of PDE10A inhibitors for the treatment of UC.

### Assessing PDE10A Inhibitors for Use in the Treatment of Crohn's Disease

As mentioned above, treatments for UC should also be viable treatments for CD. In particular, it has been shown that cGMP signalling is reduced in both UC and CD (Brenna, et al. The guanylate cyclase-C signaling pathway is down-regulated in inflammatory bowel disease. Scand J Gastroentero 50, 1241-1252 (2015)) a mechanism that is highly relevant to PDE10A.

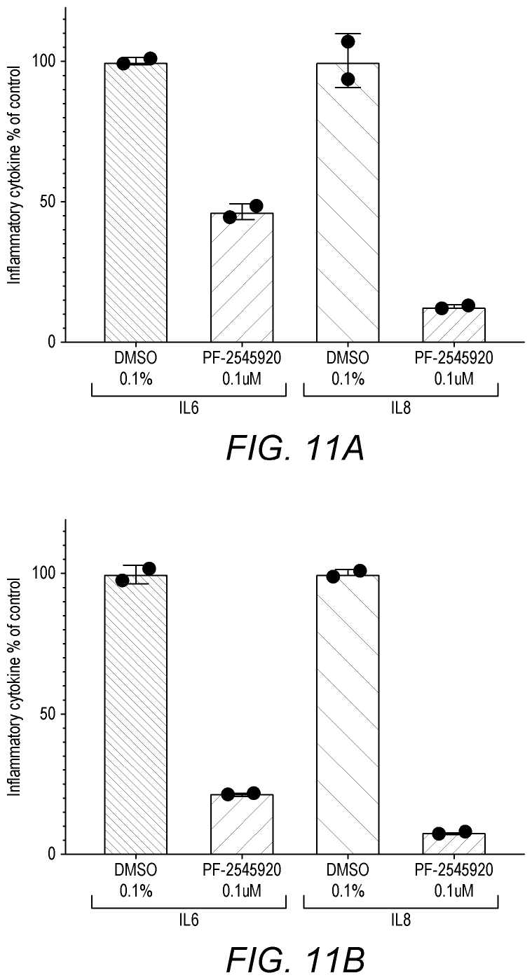

In addition, we tested the effect of selective PDE10A inhibition using 0.1 uM PF-2545920 on inflamed colonic mucosa from Crohn's Disease patients taken during routine endoscopy (Protocol 1). Selective PDE10A inhibition significantly reduced the secreted levels of IL-6 and IL-8 from 2 independent CD patient biopsies when compared to the DMSO vehicle (FIG. 11).

The ability of selective PDE10A inhibition to significantly reduce levels of inflammatory cytokines in CD patient-derived colonic mucosa demonstrates the therapeutic utility of PDE10A inhibitors for the treatment of CD in addition to UC.

As PF-2545920 may treat CD by inhibition of PDE10A, the compounds of the invention may also treat CD.

### Protocol 1

Biopsy tissue was obtained from inflamed colonic mucosa from ulcerative colitis or Crohn's disease patients during routine endoscopy. Ex-vivo biopsy cultures for the analysis of inflammatory cytokine biomarkers were run as previously described (Vossenksmper A. et al. A CD3-specific antibody reduces cytokine production and alters phosphoprotein profiles in intestinal tissues from patients with inflammatory bowel disease. Gastroenterology, 147, 172-183 (2014)). Biopsies were incubated in organ culture for 24 h with the addition of positive control compounds, or specific PDE10A inhibitors. Supernatants collected at the end of the experiment were snap-frozen and stored at −70° C. For the measurement of cytokines, the frozen culture supernatants were thawed and analysed for levels of the inflammatory cytokines using Luminex cytokine assay kits (R&D Systems) and an R&D Systems MAGPIX® analyser. Mean values±SDs were calculated for the levels of spontaneous cytokine production measured in biopsy culture supernatants from each treatment group.

Protocol 2 Ulcerative colitis donor samples were obtained with full ethical consent from patients undergoing therapeutic resection for ulcerative colitis. Tissues were placed apical (mucosal) side facing upwards on a Netwell filter. The biopsies were then cultured in either control media or media containing the test compound in an incubator at 37° C. and high O₂ atmospheric conditions. To try to minimize variation, the biopsies were also cultured in the presence of the inflammatory stimulant Staphylococcal Enterotoxin B (SEB) to help normalise cytokine levels. At approximately 18 hours post-culture start, media samples were collected, protease inhibitor added and samples stored at −80° C. Supernatants were subsequently subjected to ELISA analysis for cytokine measurement.

### Assessing PDE10A Inhibitors in IL-8 Neutrophil Activation

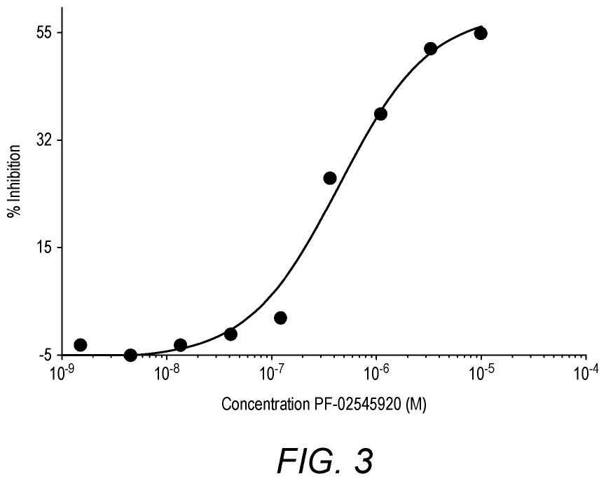

The PDE10A compound PF-02545920 was assessed in an in vitro assay of IL-8 neutrophil activation. PF-02545920 dose dependently inhibited IL-8 induced neutrophil activation (as shown in FIG. 3). This was of interest as a role for PDE10A in neutrophil function had not been previously described and further suggested a role for PDE10A in modulating inflammation and that a PDE10A inhibitor would be suitable as a therapeutic for inflammatory bowel diseases.

### Synthesis of PDE10A Inhibitors (Reference Examples) a to G

### Reference Intermediate 1

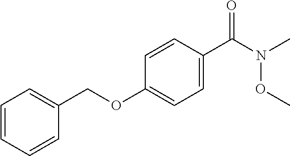 [SMILES: CON(C)C(=O)c1ccc(OCc2ccccc2)cc1]

### 4-Benzyloxy-N-methoxy-N-methyl-benzamide

Oxalyl chloride (3.76 mL, 43.8 mmol) was added dropwise to a suspension of 4-benzyloxybenzoic acid (5.00 g, 21.9 mmol) in DCM (75 mL) and DMF (400 μL) at 0° C. The reaction was warmed to room temperature, stirred for 2 h then concentrated in vacuo. The residue was dissolved in DCM (100 mL) and N,O-dimethylhydroxylamine hydrochloride (2.14 g, 21.9 mmol) was added. The reaction was cooled to 0° C. and TEA (7.63 mL, 54.8 mmol) was added dropwise then warmed to room temperature and stirred for 18 h. The reaction mixture was partitioned between DCM (250 mL) and sat aq NaHCO₃ (250 mL). The aq layer was extracted with DCM (250 mL) and the organic layers combined, washed with brine (250 mL), dried (MgSO₄) and concentrated in vacuo. The residue was purified by normal phase column chromatography to give the title compound (4.53 g, 76.3%) as a white solid. UPLC (Method F) Rt 5.53 min, 100%. LCMS (ES+): 272.1 [MH]⁺.

### Reference Intermediate 2

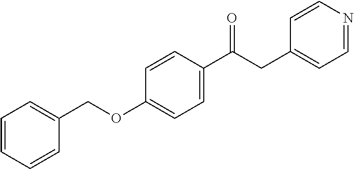 [SMILES: O=C(Cc1ccncc1)c1ccc(OCc2ccccc2)cc1]

### 1-(4-Benzyloxyphenyl)-2-(4-pyridyl)ethanone

Under N₂ n-BuLi (2.5M in hexanes, 13.4 mL, 33.4 mmol) was added dropwise to a solution of diisopropylamine (4.71 mL, 33.4 mmol) in THF (40 mL) at −78° C. The reaction was stirred for 30 min, warmed to 0° C. and stirred for 30 min. 4-Methylpyridine (3.28 mL, 33.4 mmol) was added dropwise and the reaction was stirred for 30 min. In a separate flask under N₂, reference intermediate 1 (4.53 g, 16.7 mmol) was dissolved in THF (100 mL) and cooled to −78° C. The solution of 4-methylpyrine anion was added dropwise over 1 h. The reaction was stirred for 1 h. AcOH (20 mL) was added, and the reaction was allowed to warm to room temperature overnight. The reaction mixture was concentrated in vacuo then partitioned between DCM (250 mL) and water (250 mL). The aq portion was extracted with DCM (250 mL). The combined organic portions were washed with sat NaHCO₃ (250 mL), dried (MgSO₄) and concentrated in vacuo to give the title compound (4.85 g, 93.0%) as a light yellow solid. UPLC (Method F) Rt 4.67 min, 97.2%. LCMS (ES+): 304.2 [MH]⁺.

### Reference Intermediate 3 (and by-Product)

### 4-[3-(4-Benzyloxyphenyl)-1-methyl-pyrazol-4-yl]pyridine and 4-[5-(4-benzyloxyphenyl)-1-methyl-pyrazol-4-yl]pyridine (by-product)

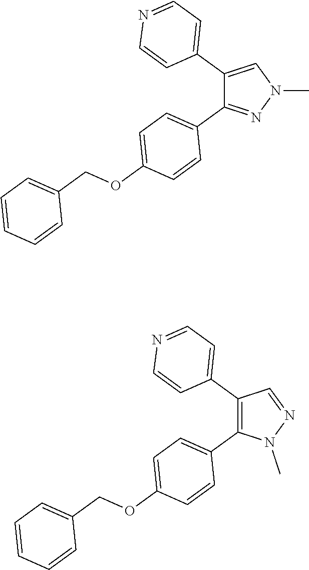 [SMILES: Cn1cc(-c2ccncc2)c(-c2ccc(OCc3ccccc3)cc2)n1 ; Cn1ncc(-c2ccncc2)c1-c1ccc(OCc2ccccc2)cc1]

Reference intermediate 2 (3.85 g, 97.2% pure, 12.3 mmol) in DMFDMA (25 mL) was heated at reflux for 2 h then concentrated in vacuo. The residue was dissolved in EtOH (60 mL), methylhydrazine (1.95 mL, 37.0 mmol) and conc sulfuric acid (138.3 μL, 2.46 mmol) were added and the reaction was heated at 70° C. for 3 h. The reaction mixture was concentrated in vacuo then partitioned between DCM (250 mL) and sat aq NaHCO₃ (250 mL). The aq layer was extracted with DCM (250 mL) and the organic layers combined, dried (MgSO₄) and concentrated in vacuo. The residue was purified by normal phase column chromatography (1% TEA buffered) to give the title compounds (2.84 g, 65.9%) as a yellow solid and (625 mg, 10.7%) as a yellow solid respectively. UPLC Rt 4.67 min, 97.6%. LCMS (ES+): 342.2 [MH]+. UPLC (Method F) 4.74 min, 72.2%. LCMS (ES+): 342.3 [MH]+.

### Reference Intermediate 4

### 4-[1-Methyl-4-(4-pyridyl)pyrazol-3-yl]phenol

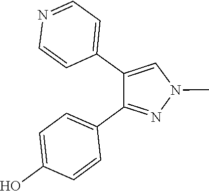 [SMILES: Cn1cc(-c2ccncc2)c(-c2ccc(O)cc2)n1]

Reference intermediate 3 (3.48 g, 97.6% pure, 9.95 mmol) was dissolved in EtOH (100 mL) and EtOAc (100 mL) and the solution was passed through an H-cube (70×4 mm 10% Pd/C CatCart, 1.0 mL/min, 60° C., 50 bar) twice. The reaction mixture was concentrated in vacuo to give the title compound (2.57 g, 98.9%) as a white solid. UPLC (Method F) Rt 2.73 min, 96.1%. LCMS (ES+): 252.1 [MH]⁺.

### Reference Intermediate 5

### tert-Butyl 2-methylquinoline-4-carboxylate

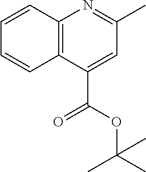 [SMILES: Cc1cc(C(=O)OC(C)(C)C)c2ccccc2n1]

N,N′-Dicyclohexylcarbodiimide (1.65 g, 8.01 mmol) was added portion wise to a suspension of 2-methylquinoline-4-carboxylic acid (1.00 g, 5.34 mmol), DMAP (65.3 mg, 534 μmol) and tert-butanol (1.02 mL, 10.7 mmol) in DCM (60 mL) at the mixture stirred for 16 h. The reaction mixture was filtered and concentrated in vacuo. The residue was purified by normal phase column chromatography to give the title compound (539 mg, 41.2%) as a yellow oil. UPLC (Method F) Rt 4.31 min, 99.4%. LCMS (ES+): 244.2 [MH]⁺.

### Reference Intermediate 6

### Methyl 2-(bromomethyl)quinoline-3-carboxylate

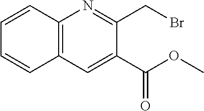 [SMILES: COC(=O)c1cc2ccccc2nc1CBr]

Azobisisobutyronitrile (44.1 mg, 269 μmol) was added to a solution of methyl 2-methylquinoline-3-carboxylate (548 mg, 98.8% pure, 2.69 mmol) and NBS (718 mg, 4.03 mmol) in CCl₄ (13 mL) and the reaction heated at reflux for 4 h. The reaction mixture was filtered and concentrated in vacuo then purified by normal phase column chromatography to give the title compound (492 mg, 60.9%) as a yellow solid. UPLC (Method F) Rt 5.55 min, 93.2%. LCMS (ES+): 280.0 [MH]⁺.

### Reference Intermediates 7A and 7B

Reference intermediates 7A and 7B were prepared similarly to reference intermediate 6, by bromination of the appropriate intermediate with NBS.

| Ref | | | Intermediate, Form, |
|---|---|---|---|
| Int | Structure | Name | Yield, LCMS, UPLC |
| 7A | 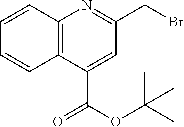 [SMILES: CC(C)(C)OC(=O)c1cc(CBr)nc2ccccc12] | tert-Butyl 2- (bromomethyl) quinoline-4-carboxylate | From reference intermediate 5 Light-yellow oil Yield 328 mg, 37.5% LCMS (ES+): 322.0 [MH]⁺. UPLC (Method F) Rt 6.85 min, 99.1%. |
| 7B | 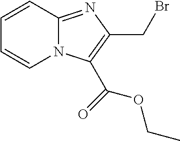 [SMILES: CCOC(=O)c1c(CBr)nc2ccccn12] | Ethyl 2- (bromomethyl)imidazo [1,2-a]pyridine-3- carboxylate | From ethyl 2- methylimidazo[1,2- a]pyridine-3-carboxylate Beige solid Yield 323 mg, 37.4% LCMS (ES+): 282.9 [MH]⁺. UPLC (Method B) Rt 2.26 min, 80.2%. |

### Reference Intermediate 8

### Ethyl 2-(chloromethyl)-1,5-naphthyridine-3-carboxylate

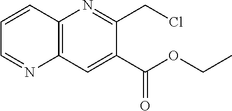 [SMILES: CCOC(=O)c1cc2ncccc2nc1CCl]

3-Aminopicolinaldehyde (500 mg, 4.09 mmol) and ethyl 4-chloro-3-oxobutanoate (0.66 mL, 4.91 mmol) were dissolved in EtOH (27 mL) and heated under reflux for 18 h. The mixture was concentrated in vacuo. The residue was separated between EtOAc (100 mL) and water (100 mL), the aq portion was extracted with EtOAc (100 mL) and the combined organics were dried (MgSO₄) and concentrated in vacuo. The residue purified by trituration in iso-hexane to give the title compound (724 mg, 69.5%) as a brown solid. UPLC (Method B) Rt 2.46 min, 98.5%. LCMS (ES+): 251.0 [MH]⁺.

### Reference Intermediate 9

### Methyl 2-[[4-[1-methyl-4-(4-pyridyl)pyrazol-3-yl]phenoxy]methyl]quinoline-3-carboxylate

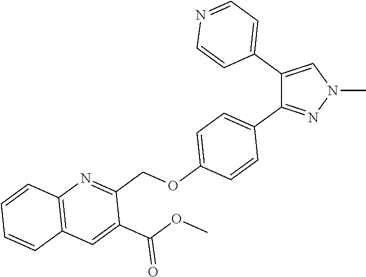 [SMILES: COC(=O)c1cc2ccccc2nc1COc1ccc(-c2nn(C)cc2-c2ccncc2)cc1]

Under N₂ a solution of reference intermediate 4 (300 mg, 96.1% pure, 1.15 mmol) in DMF (4.0 mL) was added dropwise to a suspension of NaH (60% in mineral oil, 50.5 mg, 1.26 mmol) in DMF (8.0 mL) at 0° C. and stirred for 30 min. reference intermediate 6 (345 mg, 93.2% pure, 1.15 mmol) was added and the mixture was allowed to warm to room temperature over 16 h. The reaction mixture was partitioned between DCM (100 mL), H₂O (100 mL) and brine (50 mL), the aq layer extracted with DCM (100 mL) and the organic layers combined, washed with brine (100 mL), dried (MgSO₄) and concentrated in vacuo. The residue was purified by normal phase column chromatography to give the title compound (439 mg, 82.5%) as a yellow solid. UPLC (Method F), Rt 4.52 min, 97.1%. LCMS (ES+): 451.2 [MH]⁺.

### Reference Intermediate 10

### tert-Butyl 2-[[4-[1-methyl-4-(4-pyridyl)pyrazol-3-yl]phenoxy]methyl]quinoline-4-carboxylate

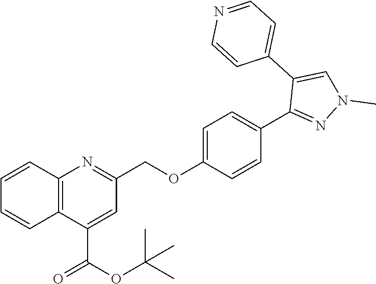 [SMILES: Cn1cc(-c2ccncc2)c(-c2ccc(OCc3cc(C(=O)OC(C)(C)C)c4ccccc4n3)cc2)n1]

Reference intermediate 10 was prepared similarly to reference intermediate 9, by alkylation of reference intermediate 4 with reference intermediate 7A using NaH; Yellow gum. Yield 500 mg, 90.1%; LCMS (ES+): 493.3 [MH]⁺; UPLC (Method F), Rt 5.23 min, 89.5%.

### Reference Intermediate 11

### 2-[[4-[1-Methyl-4-(4-pyridyl)pyrazol-3-yl]phenoxy]methyl]quinoline-3-carboxylic acid

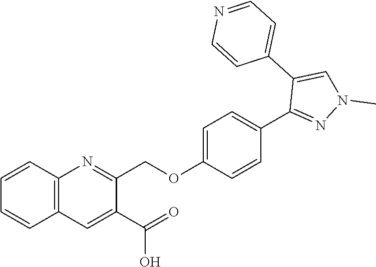 [SMILES: Cn1cc(-c2ccncc2)c(-c2ccc(OCc3nc4ccccc4cc3C(=O)O)cc2)n1]

LiOH·H₂O (119 mg, 2.84 mmol) was added to a solution of reference intermediate 9 (439 mg, 97.1% pure, 946 μmol) in THF (5.0 mL) and water (5.0 mL) and stirred for 2 h. The volatiles were removed in vacuo. To the remaining aq portion was added 1 M aq HCl (2.84 mL). The resulting solid was collected by filtration and washed with water (2×5 mL) to give 2-[[4-[1-methyl-4-(4-pyridyl)pyrazol-3-yl]phenoxy]methyl] quinoline-3-carboxylic acid (370 mg, 87.8%) as a white solid. UPLC (Method F) Rt 3.78 min, 98.0%. LCMS (ES+): 437.1 [MH]⁺.

### Reference Intermediate 12

### Ammonium 2-[[4-[1-methyl-4-(4-pyridyl)pyrazol-3-yl]phenoxy]methyl]quinoline-4-carboxylate

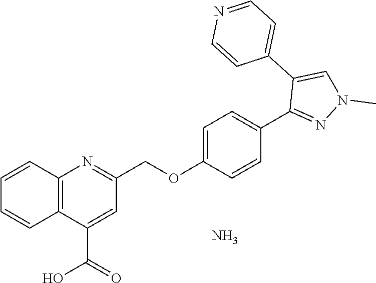

1,4-Dioxane (5.0 mL) and hydrochloric acid (4M in 1,4-dioxane, 5.0 mL, 20 mmol) was added to reference intermediate 10 (500 mg, 909 μmol) in water (5.0 mL) and the reaction heated at 60° C. for 3 h. The reaction mixture was concentrated in vacuo to give 2-[[4-[1-methyl-4-(4-pyridyl)pyrazol-3-yl]phenoxy]methyl]quinoline-4-carboxylic acid dihydrochloride (510 mg, 99.8%) as a brown solid. 50 mg was dissolved in THF (2.0 mL) and water (2.0 mL) and neutralised to pH 7 then purified by reverse phase HPLC (ammonia buffered) to give the title compound (14.8 mg, 3.58%) as a white solid. UPLC (Method F), Rt 3.77 min, 99.6%. LCMS (ES+): 437.1 [MH]⁺.

### Reference Intermediate 13

### Methyl 2-(bromomethyl)quinazoline-4-carboxylate

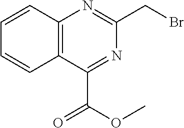 [SMILES: COC(=O)c1nc(CBr)nc2ccccc12]

Oxalyl chloride (0.19 mL, 2.23 mmol) was added to a solution of 2-methylquinazoline-4-carboxylic acid hydrochloride (250 mg, 1.11 mmol) in DCM (11 mL) and DMF (10 μL) at 0° C. The reaction was stirred for 30 min. MeOH (1.0 mL) was added and the reaction was warmed to room temperature and stirred for 30 min. The mixture was concentrated in vacuo, diluted with EtOAc (30 mL), washed with sat aq NaHCO₃ (30 mL) dried (MgSO₄) and concentrated in vacuo. The residue was dissolved in CCl₄ (5.0 mL) and the mixture was sparged with N₂ for 5 min. Azobisisobutyronitrile (15.5 mg, 9.45 μmol) and NBS (210 mg, 1.18 mmol) were added and the reaction was heated under reflux for 20 h. The mixture filtered and concentrated in vacuo. The residue was purified by normal phase column chromatography to give the title compound (58.0 mg, 20.0%) as a white solid. UPLC (Method B) Rt 2.46 min, 91.5%. LCMS (ES+): 281.0 [MH]⁺.

### Reference Intermediate 14

### Methyl 2-[[4-[1-methyl-4-(4-pyridyl)pyrazol-3-yl]phenoxy]methyl]quinazoline-4-carboxylate

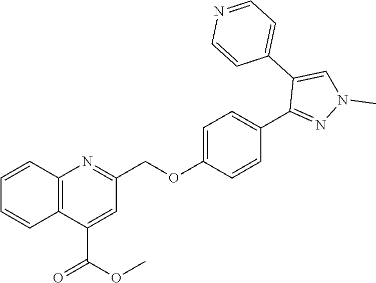 [SMILES: COC(=O)c1cc(COc2ccc(-c3nn(C)cc3-c3ccncc3)cc2)nc2ccccc12]

A mixture of reference intermediate 4 (56.1 mg, 99.2% pure, 0.22 mmol), intermediate 13 (68.0 mg, 91.5% pure, 0.22 mmol) and Cs₂CO₃ (79.3 mg, 0.24 mmol) in DMF (3.0 mL) was stirred for 16 h. The mixture was diluted with DCM (20 mL), washed with sat aq NaHCO₃ (20 mL), dried (MgSO₄) and concentrated in vacuo. The residue was purified by normal phase column chromatography to give the title compound (35.0 mg, 34.6%) as a yellow solid. UPLC (Method B) Rt 2.24 min, 98.7%. LCMS (ES+): 452.1 [MH]⁺.

### REFERENCE INTERMEDIATES 15 and 16

Reference intermediates 15 and 16 were prepared similarly to reference intermediate 14, by alkylation of the appropriate phenol intermediates with the appropriate bromide/chloride intermediates using 05₂00₃.

| | | | Intermediate(s), |
|---|---|---|---|
| Ref | | | Form, |
| Int | Structure | Name | Yield, LCMS, UPLC |
| 15 | 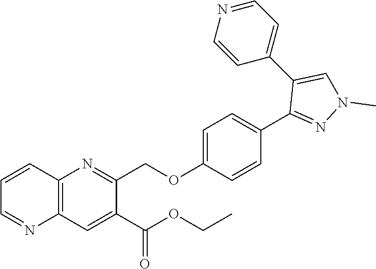 [SMILES: CCOC(=O)c1cc2ncccc2nc1COc1ccc(-c2nn(C)cc2-c2ccncc2)cc1] | Ethyl 2-[[4-[1- methyl-4-(4- pyridyl)pyrazol-3- yl]phenoxy]methyl]- 1,5- naphthyridine-3- carboxylate | From reference intermediates 4 and 8 Brown solid Yield 87.0 mg, 76.4% LCMS (ES+): 466.2 [MH]⁺ UPLC (Method B): Rt 2.24 min, 96.8% |
| 16 | 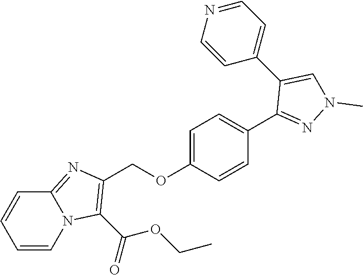 [SMILES: CCOC(=O)c1c(COc2ccc(-c3nn(C)cc3-c3ccncc3)cc2)nc2ccccn12] | Ethyl 2-[[4-[1- methyl-4-(4- pyridyl)pyrazol-3- yl]phenoxy]methyl] imidazo[1,2- a]pyridine-3- carboxylate | From reference intermediates 4 and 7B Yellow solid Yield 86 mg, 43.0% LCMS (ES+): 454.1 [MH]⁺. UPLC (Method B) Rt 2.10 min, 89.6% |

### Reference Intermediate 17

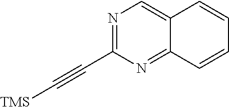 [SMILES: C[Si](C)(C)C#Cc1ncc2ccccc2n1]

### Trimethyl(2-quinazolin-2-ylethynyl)silane

Under an atmosphere of N₂, trimethylsilylacetylene (2.6 mL, 18.4 mmol) and DIPEA (3.0 mL, 17.2 mmol) were added sequentially to a suspension of 2-chloroquinoxaline (1.50 g, 9.11 mmol), CuI (174 mg, 0.91 mmol), and Pd(dppf)Cl₂—CH₂Cl₂ (1.48 g, 1.82 mmol) in DMF (15 mL). The mixture was stirred at 80° C. for 1 h before quenching with water (30 mL) and extraction with EtOAc (5×30 mL). The combined organic layers were washed with brine (50 mL), dried over anhydrous Na₂SO₄, and concentrated in vacuo. The residue was purified by silica gel column chromatography to afford the title compound (1.40 g, 65.8%) as a yellow solid. LCMS (ES+): 227.2 [MH]⁺.

### Reference Intermediate 18

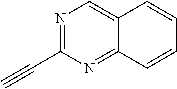

### 2-Ethynylquinazoline

A suspension of reference intermediate 17 (600 mg, 2.65 mmol) and K2C₀₃ (366 mg, 2.65 mmol) in MeOH (5.0 mL) and DCM (5.0 mL) was stirred for 1 h before the addition of water (40 mL) and extraction with EtOAc (5×40 mL). The combined organic layers were washed with brine (50 mL), dried over anhydrous Na₂SO₄, and concentrated in vacuo. The residue was purified by silica gel column chromatography to afford the title compound (360 mg, 88.1%) as a white solid. LCMS (ES+): 155.3 [MH]⁺.

### Reference Intermediate 19

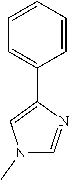

### 1-Methyl-4-phenyl-imidazole

Under an atmosphere of N₂, NaH (60% in mineral oil, 3.33 g, 83.2 mmol) was added to a solution of 4-phenyl-3H-imidazole (10 g, 69.4 mmol) in DMF (70 mL) at 0° C. The solution was stirred for 30 min before the dropwise addition of CH₃I (5.2 mL, 83.5 mmol) over 10 min while maintaining a temperature of 0° C. The resultant mixture was stirred for a further 1 h without cooling. The reaction was then quenched by the addition of water (100 mL) and extracted with EtOAc (5×100 mL). The combined organic layers were washed with brine (30 mL), dried over anhydrous Na₂SO₄, and concentrated in vacuo. The residue was purified by silica gel column chromatography to afford the title compound (7.80 g, 71.1%) as a yellow solid. LCMS (ES+): 159.0 [MH]⁺.

### Reference Intermediate 20

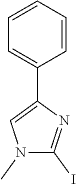

### 2-Iodo-1-methyl-4-phenyl-imidazole

Under an atmosphere of N₂, n-BuLi (2.5 M in hexanes, 29.6 mL, 74.0 mmol) was added dropwise to a solution of reference intermediate 19 (7.8 g, 49.3 mmol) in THF (80 mL) over 10 min at −78° C. The solution was stirred for 1 h at −78° C. before the dropwise addition of I₂ (13.8 g, 54.2 mmol) in THF (20 mL) over 10 min. The resultant mixture was warmed to 0° C. and stirred for an additional 1 h before quenching with water (100 mL) and extracted with EtOAc (5×100 mL). The combined organic layers were washed with brine (30 mL), dried over anhydrous Na₂SO₄, and concentrated in vacuo. The residue was purified by silica gel column chromatography to afford the title compound (7.70 g, 55.0%) as a yellow solid. LCMS (ES+): 285.0 [MH]⁺.

### Reference Intermediate 21

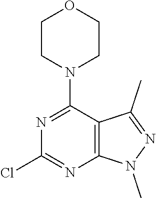 [SMILES: Cc1nn(C)c2nc(Cl)nc(N3CCOCC3)c12]

### 4-(6-Chloro-1,3-dimethyl-pyrazolo[3,4-d]pyrimidin-4-yl)morpholine

4,6-dichloro-1,3-dimethylpyrazolo[3,4-d]pyrimidine (300 mg, 1.38 mmol), TEA (578 L, 4.15 mmol) and morpholine (143 μL, 1.66 mmol) in DMF (10 mL) was stirred for 2 h at room temperature before concentration in vacuo. The residue was purified by silica gel column chromatography to afford the title compound (200 mg, 54.1%) as a yellow solid. LCMS (ES+): 268.2 [MH]⁺.

### Reference Intermediate 22

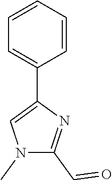

### 1-Methyl-4-phenyl-imidazole-2-carbaldehyde

Under an atmosphere of N₂, n-BuLi (2.5 M in hexanes, 1.5 mL, 3.8 mmol) was added dropwise to a solution of reference intermediate 19 (500 mg, 3.16 mmol) in THF (10 mL) over 10 min at −40° C. and stirred for 30 min before cooling to −78° C. DMF (300 L, 4.11 mmol) was added dropwise over 10 min before stirring for a further 1 h before allowing the solution to warm to RT over 2 h. The reaction was quenched with the addition of water (30 mL) and extracted with EtOAc (5×50 mL). The combined organic layers were washed with brine (3×10 mL), dried over anhydrous Na₂SO₄, and concentrated in vacuo. The residue was purified by silica gel column chromatography to afford the title compound (450 mg, 75.5%) as a white solid. LCMS (ES+): 187.3 [MH]⁺.

### Reference Intermediate 23

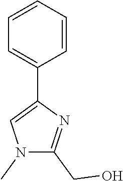

### (1-Methyl-4-phenyl-imidazol-2-yl)methanol

NaBH₄ (183 mg, 4.84 mmol) was added to a solution of reference intermediate 22 (450 mg, 2.42 mmol) in MeOH (5.0 mL) and stirred for 1 h before the addition of water (20 mL) and extraction with EtOAc (5×30 mL). The combined organic layers were washed with brine (3×10 mL), dried over anhydrous Na₂SO₄, and concentrated in vacuo. The residue was purified by silica gel column chromatography to afford the title compound (440 mg, 96.7%) as a white solid. LCMS (ES+): 189.2 [MH]⁺.

The following compounds are reference examples.

### Reference Example A

### Ammonium 2-[[4-[1-methyl-4-(4-pyridyl)pyrazol-3-yl]phenoxy]methyl]quinoline-4-carboxylate

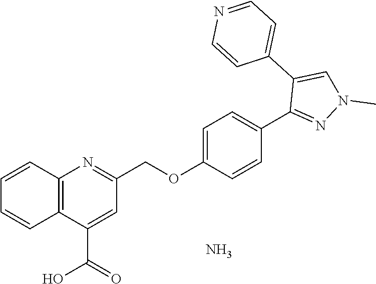

1,4-Dioxane (5.0 mL) and hydrochloric acid (4M in 1,4-dioxane, 5.0 mL, 20 mmol) was added to reference intermediate 10 (500 mg, 909 μmol) in water (5.0 mL) and the reaction heated at 60° C. for 3 h. The reaction mixture was concentrated in vacuo to give 2-[[4-[1-methyl-4-(4-pyridyl)pyrazol-3-yl]phenoxy]methyl]quinoline-4-carboxylic acid dihydrochloride (510 mg, 99.8%) as a brown solid. 50 mg was dissolved in THF (2.0 mL) and water (2.0 mL) and neutralised to pH 7 then purified by reverse phase HPLC (ammonia buffered) to give the title compound (14.8 mg, 3.58%) as a white solid. UPLC (Method F), Rt 3.77 min, 99.6%. LCMS (ES+): 437.1 [MH]⁺.

### Reference Example B

### 2-[[4-[1-Methyl-4-(4-pyridyl)pyrazol-3-yl]phenoxy]methyl]-N-methylsulfonyl-quinoline-3-carboxamide

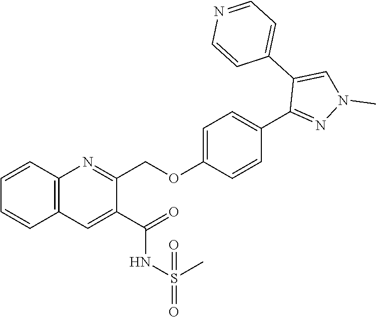 [SMILES: Cn1cc(-c2ccncc2)c(-c2ccc(OCc3nc4ccccc4cc3C(=O)NS(C)(=O)=O)cc2)n1]

DIPEA (58.7 μL, 337 μmol) was added to a suspension of reference intermediate 11 (50.0 mg, 98.0% pure, 112 μmol), methanesulfonamide (21.4 mg, 224 μmol) and HATU (85.4 mg, 224 μmol) in DCM (1.2 mL) and stirred for 6.5 h. The reaction mixture was partitioned between DCM (3 mL) and H₂O (3 mL). The aq layer was extracted with DCM (30 mL) and the organic layers combined, dried (MgSO₄) and concentrated in vacuo. The residue was purified by reverse phase HPLC to give the title compound (18.8 mg, 32.1%) as a white solid. UPLC (Method F), Rt 3.96 min, 98.4%. LCMS (ES+): 514.1 [MH]⁺.

### Reference Examples C and D

Reference examples C and D were prepared similarly to reference example B, by amide coupling of the appropriate intermediates with the appropriate amines using HATU.

| Ref | | | Intermediate, Form, |
|---|---|---|---|
| Ex | Structure | Name | Yield, LCMS, UPLC |
| C | 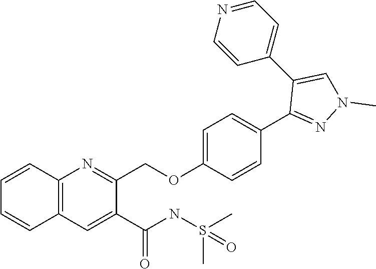 | N-[Dimethyl(oxo)- λ6-sulfanylidene]-2- [[4-[1-methyl-4-(4- pyridyl)pyrazol-3- yl]phenoxy]methyl] quinoline-3- carboxamide | From reference intermediate 11 White solid Yield 32.6 mg, 56.1% LCMS (ES+): 512.1 [MH]⁺ UPLC (Method F): Rt 3.71 min, 98.9% |
| D | 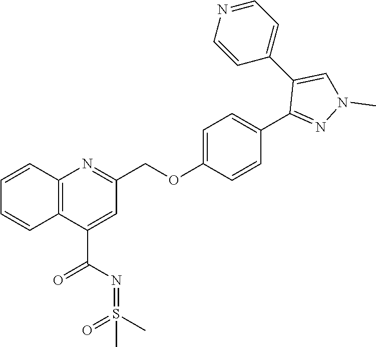 [SMILES: Cn1cc(-c2ccncc2)c(-c2ccc(OCc3cc(C(=O)N=S(C)(C)=O)c4ccccc4n3)cc2)n1] | N-[Dimethyl(oxo)- λ6-sulfanylidene]-2- [4-[1-methyl-4-(4- pyridyl)pyrazol-3- yl]phenoxy]methyl] quinoline-4- carboxamide | From reference intermediate 12 White solid Yield 165 mg, 40.6% LCMS (ES+): 512.1 [MH]⁺ UPLC (Method F): Rt 3.82 min, 100% |

### Reference Example E

### N-[Dimethyl(oxo)-λ6-sulfanylidene]-2-[[4-[1-methyl-4-(4-pyridyl)pyrazol-3-yl]phenoxy]methyl]quinazoline-4-carboxamide

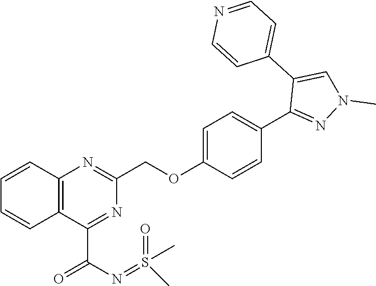 [SMILES: Cn1cc(-c2ccncc2)c(-c2ccc(OCc3nc(C(=O)N=S(C)(C)=O)c4ccccc4n3)cc2)n1]

Reference intermediate 14 (35.0 mg, 98.7% pure, 76.5 μmol) was dissolved in THF (2.0 mL) and water (2.0 mL). LiOH·H₂O (3.85 mg, 91.8 μmol) was added and the reaction was stirred for 1 h. The mixture was concentrated in vacuo. The residue was dissolved in DMF (2.0 mL), HATU (58.2 mg, 0.15 mmol), S,S-dimethyl sulfoximine (14.3 mg, 0.15 mmol) and DIPEA (26.7 μL, 0.15 mmol) were added and the reaction was stirred for 1.5 h. The mixture was diluted with DCM (30 mL), washed with sat aq NaHCO₃ (30 mL), dried (MgSO₄) and concentrated in vacuo. The residue was purified by reverse phase HPLC (NH₃ buffered) to give the title compound (24.4 mg, 61.8%) as a yellow solid. UPLC (Method F) Rt 3.68 min, 99.4%. LCMS (ES+): 513.1 [MH]⁺.

### Reference Examples F and G

Reference examples F and G were prepared similarly to reference example E, by saponification of the appropriate reference intermediates followed by amide coupling with the appropriate amines using HATU.

| Ref | | | Intermediate, Form, |
|---|---|---|---|
| Ex | Structure | Name | Yield, LCMS, UPLC |
| F | 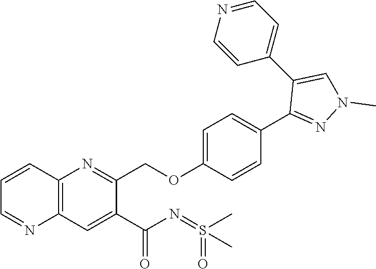 [SMILES: Cn1cc(-c2ccncc2)c(-c2ccc(OCc3nc4cccnc4cc3C(=O)N=S(C)(C)=O)cc2)n1] | N-[Dimethyl(oxo)- λ6-sulfanylidene]-2- [4-[1-methyl-4-(4- pyridyl)pyrazol-3- yl]phenoxy]methyl]- 1,5-naphthyridine- 3-carboxamide | From reference intermediate 15 Yellow solid Yield 49.9 mg, 53.8% LCMS (ES+): 513.1 [MH]⁺ UPLC (Method F): Rt 3.44 min, 100% |
| G | 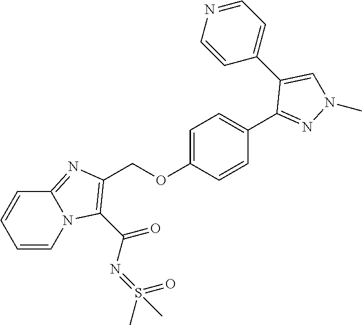 [SMILES: Cn1cc(-c2ccncc2)c(-c2ccc(OCc3nc4ccccn4c3C(=O)N=S(C)(C)=O)cc2)n1] | N-[Dimethyl(oxo)- λ6-sulfanylidene]-2- [[4-[1-methyl-4-(4- pyridyl)pyrazol-3- yl]phenoxy]methyl] imidazo[1,2- alpyridine-3- carboxamide | From reference intermediate 16 Pink solid Yield 19.4 mg, 22.5% LCMS (ES+): 501.3 [MH]⁺. UPLC (Method F) Rt 3.09 min, 98.7%. |

### Reference Example H

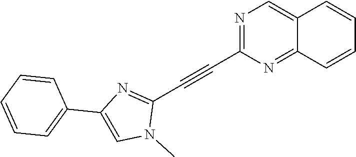 [SMILES: Cn1cc(-c2ccccc2)nc1C#Cc1ncc2ccccc2n1]

### 2-[2-(1-Methyl-4-phenyl-imidazol-2-yl)ethynyl]quinazoline

Under an atmosphere of N₂, a mixture of reference intermediate 18 (150 mg, 0.97 mmol), reference intermediate 20 (332 mg, 1.17 mmol), 2nd Generation XantPhos precatalyst (86 mg, 0.10 mmol), Xanthphos (56 mg, 0.10 mmol), CuI (9.00 mg, 0.05 mmol), and TEA (500 μL, 3.63 mmol) in DMF (5.0 mL) was stirred for 1 h before the addition of water (20 mL) and extraction with EtOAc (5×30 mL). The combined organic layers were concentrated in vacuo and the residue was purified by reverse phase HPLC (HCOOH buffered) to afford the title compound (27.9 mg, 9.21%) as a brown solid. UPLC (Method E): Rt 1.39 min. LCMS (ES+): 311.10 [MH]⁺.

### Reference Example J

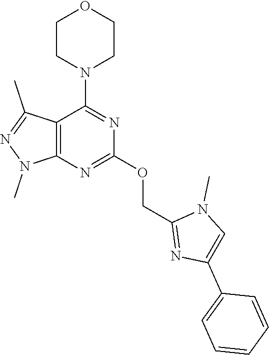 [SMILES: Cc1nn(C)c2nc(OCc3nc(-c4ccccc4)cn3C)nc(N3CCOCC3)c12]

### 4-[1,3-Dimethyl-6-[(1-methyl-4-phenyl-imidazol-2-yl)methoxy]pyrazolo[3,4-d]pyrimidin-4-yl]morpholine

A suspension of reference intermediate 23 (150 mg, 0.80 mmol), reference intermediate 21 (200 mg, 0.80 mmol), and Cs₂CO₃ (520 mg, 1.57 mmol) in DMF (3.0 mL) was stirred for 16 h at 100° C. before the addition of water (20 mL) and extraction with EtOAc (5×30 mL). The combined organic layers were washed with brine (3×10 mL), dried over anhydrous Na₂SO₄, and concentrated in vacuo. The residue was purified by reverse phase HPLC (NH₄HCO₃ buffered) to afford the title compound (104 mg, 32.3%) as awhite solid. UPLC (Method E): Rt 1.50 min. LCMS (ES+): 402.1 [MH]⁺.

The Reference Examples have the following activity in the human PDE10A activity assay and the cell based human PDE10A activity assay mentioned above.

| | | cAMP HTRF Assay |
|---|---|---|
| | PDE10A2 Phosphate | in HEK 293 |
| | Sensor Enzyme Assay | rhPDE10A2 cell line |
| Reference Example | (pIC₅₀) | (pIC₅₀) |
| A | 10.1 | 9.09 |
| B | 10.1 | 8.78 |
| C | 10 | 8.96 |
| D | 10.1 | 9.00 |
| E | 9.92 | 8.52 |
| F | 9.43 | 8.27 |
| G | 9.76 | 8.17 |

The data in the table above show that the reference examples are potent PDE10A inhibitors, and may therefore be suitable for use in the treatment of inflammatory bowel diseases, such as ulcerative colitis and/or Crohn's disease.

The forgoing embodiments are not intended to limit the scope of the protection afforded by the claims, but rather to describe examples of how the invention may be put into practice.

1. A compound of Formula (I) 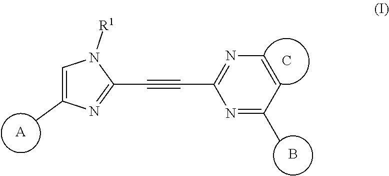
or a pharmaceutically acceptable salt, solvate, hydrate, tautomer, optical isomer, N-oxide, and/or prodrug thereof, wherein
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
Ring C is a 5- or 6-membered carbocycle, 5- or 6-membered heterocycle or 5- or 6-membered heteroaryl group, optionally substituted with one or more R²;
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle;
each R² is independently selected from the group consisting of halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
R³ is a selected from H and (C₁-C₄)alkyl, wherein the (C₁-C₄)alkyl is optionally substituted with one or more substituents selected from halo, —OH, and —NR¹⁵R¹⁶; and
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl.

2. The compound as claimed in claim 1, wherein the compound is of Formula (IA) 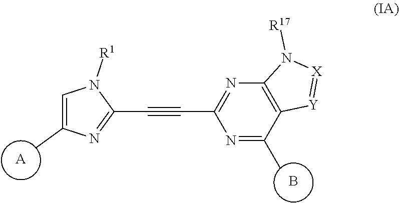
or a pharmaceutically acceptable salt, solvate, hydrate, tautomer, optical isomer, N-oxide, and/or prodrug thereof, wherein
X is N or CR¹⁸;
Y is N or CR¹⁸, preferably wherein one of X and Y is N and the other is CR¹⁸;
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo;
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle;
R³ is a selected from H and (C₁-C₄)alkyl, wherein the (C₁-C₄)alkyl is optionally substituted with one or more substituents selected from halo, —OH, and —NR¹⁵R¹⁶;
R¹⁷ is selected from the group consisting of H, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
each R¹⁸ is independently selected from the group consisting of H, halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle, preferably each R¹⁸ is H or halo, more preferably each R¹⁸ is H; and
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl.

3. The compound as claimed in claim 2, wherein Ring B is
(i) a 6- to 10-membered bicyclic heterocycle, preferably a 7- to 10-membered bicyclic heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo; or
(ii) a 4- to 7-membered monocyclic heterocycle substituted with at least one of the substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo.

4. The compound as claimed in claim 2 or claim 3, wherein Ring B is
(i) a 7- to 10-membered bicyclic heterocycle, optionally substituted with one or more substituents selected from halo, (C₁-C₄)alkyl, and —S(O)(NR⁷)R⁷, wherein the (C₁-C₄)alkyl is optionally substituted with one or more halo; or
(ii) a 4- to 7-membered monocyclic heterocycle substituted with at least one of the substituents selected from halo, (C₁-C₄)alkyl, and —S(O)(NR⁷)R⁷, wherein the (C₁-C₄)alkyl is optionally substituted with one or more halo.

5. The compound as claimed in any one of claims 1 to 4, wherein
Ring A is selected from the group consisting of aryl and 5- or 6-membered heteroaryl, each of which is optionally substituted with one or more substituents selected from halo, (C₁-C₄)alkyl, and —OR³, wherein the (C₁-C₄)alkyl is optionally substituted with one or more halo;
R³ is a selected from H and (C₁-C₄)alkyl, wherein the (C₁-C₄)alkyl is optionally substituted with one or more substituents selected from halo, and —NR¹⁵R¹⁶;
R¹⁷ is selected from the group consisting of H, (C₁-C₄)alkyl, and 4- to 6-membered heterocycle, wherein the (C₁-C₄)alkyl is optionally substituted with one or more substituents selected from halo and 4- to 6-membered heterocycle;
each R¹⁸ is independently selected from the group consisting of H, halo, and (C₁-C₄)alkyl, wherein the (C₁-C₄)alkyl is optionally substituted with one or more substituents selected from halo, preferably each R¹⁸ is H or halo, more preferably each R¹⁸ is H; and
each R⁷, R¹⁵, and R¹⁶ is independently selected from H and (C₁-C₄)alkyl, wherein the (C₁-C₄)alkyl is optionally substituted with one or more substituents selected from halo.

6. The compound as claimed in any one of claims 2 to 5, wherein Ring A is selected from the group selected from aryl and pyridyl, optionally substituted with one or more halo and —OR³, wherein R³ is (C₁-C₄)alkyl-NMe₂, preferably Ring A is selected from

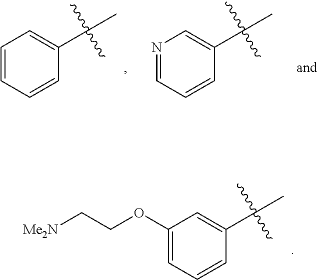 [SMILES: *c1ccccc1 ; *c1cccnc1 ; *c1cccc(OCCN(C)C)c1]

7. The compound as claimed in any one of claims 1 to 6, wherein Ring B is selected from the group consisting of 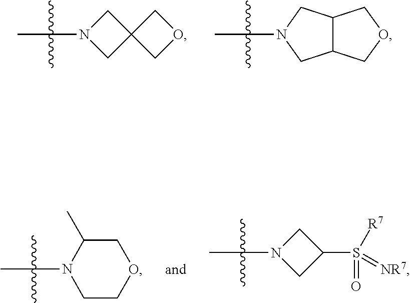
each of which is optionally substituted with one or more halo.

8. The compound as claimed in claim 1, wherein Ring C is a 5- or 6-membered heterocycle, optionally substituted with one or more R², preferably Ring C is a 6-membered heterocycle, optionally substituted with one or more R².

9. The compound as claimed in claim 8, wherein the compound is of Formula (IB) 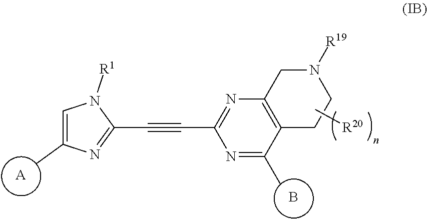
or a pharmaceutically acceptable salt, solvate, hydrate, tautomer, optical isomer, N-oxide, and/or prodrug thereof, wherein
Ring A is selected from the group consisting of aryl and 5- or 6-membered heteroaryl, each of which is optionally substituted with one or more substituents selected from halo, (C₁-C₄)alkyl, —OR³, —NR⁴R⁵, —C(O)R⁶, —C(O)OR⁶, and —C(O)NR⁴R⁵, wherein the (C₁-C₄)alkyl is optionally substituted with one or more halo,
Ring B is a 4- to 7-membered monocyclic carbocycle or heterocycle, or a 6- to 10-membered bicyclic carbocycle or heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo;
R¹ is selected from the group consisting of H and (C₁-C₄)alkyl, wherein the (C₁-C₄)alkyl is optionally substituted with one or more substituents selected from halo, —NR¹⁰R¹¹, and 4- to 6-membered heterocycle
R³ is a selected from H and (C₁-C₄)alkyl, wherein the (C₁-C₄)alkyl is optionally substituted with one or more substituents selected from halo, —OH, and —NR¹⁵R¹⁶; and
R¹⁹ is selected from the group consisting of H, (C₁-C₄)alkyl, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4-to 6-membered heterocycle;
each R²⁰ is independently selected from the group consisting of halo, (C₁-C₄)alkyl, —OR¹², —NR¹³R¹⁴, —C(O)R¹², —C(O)OR¹², —C(O)NR¹³R¹⁴, aryl, and 4- to 6-membered heterocycle, wherein the (C₁-C₄)alkyl is optionally substituted with one or more substituents selected from halo and 4- to 6-membered heterocycle;
each R⁴ to R¹⁶ is independently selected from H and (C₁-C₄)alkyl, wherein the (C₁-C₄)alkyl is optionally substituted with one or more substituents selected from halo, or groups R⁴ and R⁵, R⁸ and R⁹, R¹⁰ and R¹¹, R¹³ and R¹⁴, R¹⁵ and R¹⁶ are taken together with the atom to which they are attached to form a 5- or 6-membered heterocycle that is optionally substituted with one or more substituents selected from halo and (C₁-C₄)alkyl; and
n is 0, 1, 2, 3, 4, 5, or 6.

10. the compound as claimed in claim 9, wherein
R¹⁹ is selected from the group consisting of H, (C₁-C₄)alkyl, and 4- to 6-membered heterocycle, wherein the (C₁-C₄)alkyl is optionally substituted with one or more substituents selected from halo;
each R²⁰ is independently selected from the group consisting of halo, and (C₁-C₄)alkyl, wherein the (C₁-C₄)alkyl is optionally substituted with one or more substituents selected from halo; and
n is 0, 1, 2, 3, or 4, preferably n is 0, 1, or 2, most preferably, n is 0.

11. the compound as claimed in claim 9 or claim 10, wherein R¹⁹ is selected from the group consisting of H, (C₁-C₄)alkyl, and

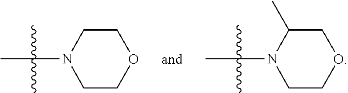

12. The compound as claimed in any one of claims 8 to 11, wherein Ring A is selected from the group consisting of aryl and 6-membered heteroaryl, each of which is optionally substituted with one or more substituents selected from halo, preferably wherein Ring A is aryl or pyridyl, more preferably aryl, or 2-pyridyl, and most preferably aryl.

13. The compound as claimed in claim any one of claims 8 to 12, wherein Ring B is a 4- to 7-membered monocyclic carbocycle or heterocycle, preferably a 4- to 7-membered monocyclic heterocycle, optionally substituted with one or more substituents selected from halo, oxo, (C₁-C₄)alkyl, —OR⁷, —NR⁸R⁹, —C(O)R⁷, —C(O)OR⁷, —C(O)NR⁸R⁹, and —S(O)(NR⁷)R⁷, wherein the (C₁-C₄)alkyl is optionally substituted with one or more halo.

14. The compound as claimed in claim 13, wherein
Ring B is a 6-membered heterocycle, optionally substituted with one of more substituents selected from halo, and (C₁-C₄)alkyl, wherein the (C₁-C₄)alkyl is optionally substituted with one or more halo.

15. The compound as claimed in claim 14, wherein
Ring B is morpholine, preferably 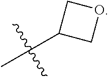 [SMILES: *C1COC1]
optionally substituted with one or more substituents selected from halo, and (C₁-C₄)alkyl, wherein the (C₁-C₄)alkyl is optionally substituted with one or more halo, most preferable, Ring B is selected from the group consisting of

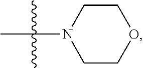 [SMILES: *N1CCOCC1]

16. The compound as claimed in claim any one of claims 8 to 15, wherein
R¹ is selected from the group consisting of H and (C₁-C₄)alkyl, wherein the (C₁-C₄)alkyl is optionally substituted with one or more substituents selected from halo, preferably wherein R¹ is H, Me or —CF₃.

17. The compound of claim 1, wherein the compound is selected from the group consisting of
6-[9-Methyl-2-[2-(1-methyl-4-phenyl-imidazol-2-yl)ethynyl]purin-6-yl]-2-oxa-6-azaspiro[3.3]heptane;
6-[9-Methyl-2-[2-[1-methyl-4-(3-pyridyl)imidazol-2-yl]ethynyl]purin-6-yl]-2-oxa-6-azaspiro[3.3]heptane;
6-[1-Methyl-6-[2-(1-methyl-4-phenyl-imidazol-2-yl)ethynyl]pyrazolo[3,4-d]pyrimidin-4-yl]-2-oxa-6-azaspiro[3.3]heptane;
6-[9-Methyl-2-[2-[4-phenyl-1-(tetrahydropyran-4-ylmethyl)imidazol-2-yl]ethynyl]purin-6-yl]-2-oxa-6-azaspiro[3.3]heptane;
6-[9-Methyl-2-[2-[1-(2-morpholinoethyl)-4-phenyl-imidazol-2-yl]ethynyl]purin-6-yl]-2-oxa-6-azaspiro[3.3]heptane;
6-[6-[2-(1-Methyl-4-phenyl-imidazol-2-yl)ethynyl]-1-tetrahydropyran-4-yl-pyrazolo[3,4-d]pyrimidin-4-yl]-2-oxa-6-azaspiro[3.3]heptane;
6-[9-Methyl-2-[2-(4-phenyl-1H-imidazol-2-yl)ethynyl]purin-6-yl]-2-oxa-6-azaspiro[3.3]heptane;
6-[1-(Azetidin-3-ylmethyl)-6-[2-(1-methyl-4-phenyl-imidazol-2-yl)ethynyl]pyrazolo[3,4-d]pyrimidin-4-yl]-2-oxa-6-azaspiro[3.3]heptane;
(3aS,6aR)-5-[1-methyl-6-[2-(4-phenyl-1H-imidazol-2-yl)ethynyl]pyrazolo[3,4-d]pyrimidin-4-yl]-1,3,3a,4,6,6a-hexahydrofuro[3,4-c]pyrrole;
2-[3-[2-[2-[4-[(3aS,6aR)-1,3,3a,4,6,6a-Hexahydrofuro[3,4-c]pyrrol-5-yl]-1-methyl-pyrazolo[3,4-d]pyrimidin-6-yl]ethynyl]-1H-imidazol-4-yl]phenoxy]-N,N-dimethyl-ethanamine;
4-[2-[2-(1-Methyl-4-phenyl-imidazol-2-yl)ethynyl]-5,6,7,8-tetrahydropyrido[3,4-d]pyrimidin-4-yl]morpholine;
(3R)-3-Methyl-4-[2-[2-(1-methyl-4-phenyl-imidazol-2-yl)ethynyl]-5,6,7,8-tetrahydropyrido[3,4-d]pyrimidin-4-yl]morpholine;
(3S)-3-Methyl-4-[2-[2-(1-methyl-4-phenyl-imidazol-2-yl)ethynyl]-5,6,7,8-tetrahydropyrido[3,4-d]pyrimidin-4-yl]morpholine;
4-[2-[2-(1-Methyl-4-phenyl-imidazol-2-yl)ethynyl]-7-(oxetan-3-yl)-6,8-dihydro-5H-pyrido[3,4-d]pyrimidin-4-yl]morpholine;
4-[7-(Cyclopropylmethyl)-2-[2-(1-methyl-4-phenyl-imidazol-2-yl)ethynyl]-6,8-dihydro-5H-pyrido[3,4-d]pyrimidin-4-yl]morpholine;
(3aR,6aS)-5-(1-(Cyclopropylmethyl)-6-((1-methyl-4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(1-Ethyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(1-isoPropyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(6-((1-Methyl-4-phenyl-1H-imidazol-2-yl)ethynyl)-1-(oxetan-3-yl)-1H-pyrazolo[3,4-d]pyrimidin-4-yl)hexahydro-1H-furo[3,4-c]pyrrole;
(3aR,6aS)-5-(9-Methyl-2-((1-methyl-4-phenyl-1H-imidazol-2-yl)ethynyl)-9H-purin-6-yl)hexahydro-1H-furo[3,4-c]pyrrole;
(R)-3-Methyl-4-(9-methyl-2-((1-methyl-4-phenyl-1H-imidazol-2-yl)ethynyl)-9H-purin-6-yl)morpholine;
(S)-3-Methyl-4-(9-methyl-2-((1-methyl-4-phenyl-1H-imidazol-2-yl)ethynyl)-9H-purin-6-yl)morpholine;
(3aR,6aS)-5-(1-Methyl-6-((1-methyl-4-phenyl-1H-imidazol-2-yl)ethynyl)-1H-pyrazolo[3,4-d]pyrimidin-4-yl)hexahydro-1H-furo[3,4-c]pyrrole;
1-Imino-4-(1-methyl-6-((1-methyl-4-phenyl-1H-imidazol-2-yl)ethynyl)-1H-pyrazolo[3,4-d]pyrimidin-4-yl)-116-thiomorpholine-1-oxide;
(3aR,6aS)-5-(1-isoPropyl-6-((4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(1-(Cyclopropylmethyl)-6-((4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(1-(Oxetan-3-yl)-6-((4-phenyl-1H-imidazol-2-yl)ethynyl)-1H-pyrazolo[3,4-d]pyrimidin-4-yl)hexahydro-1H-furo[3,4-c]pyrrole;
(3aR,6aS)-5-(9-Methyl-2-((4-phenyl-1H-imidazol-2-yl)ethynyl)-9H-purin-6-yl)hexahydro-1H-furo[3,4-c]pyrrole;
(R)-3-Methyl-4-(9-methyl-2-((4-phenyl-1H-imidazol-2-yl)ethynyl)-9H-purin-6-yl)morpholine;
(S)-3-Methyl-4-(9-methyl-2-((4-phenyl-1H-imidazol-2-yl)ethynyl)-9H-purin-6-yl)morpholine;
(3aR,6aS)-5-(9-Methyl-2-((4-(pyridin-3-yl)-1H-imidazol-2-yl)ethynyl)-9H-purin-6-yl)hexahydro-1H-furo[3,4-c]pyrrole;
(R)-3-Methyl-4-(9-methyl-2-((4-(pyridin-3-yl)-1H-imidazol-2-yl)ethynyl)-9H-purin-6-yl)morpholine; and
(S)-3-Methyl-4-(9-methyl-2-((4-(pyridin-3-yl)-1H-imidazol-2-yl)ethynyl)-9H-purin-6-yl)morpholine,
or a pharmaceutically acceptable salt, solvate, hydrate, tautomer, optical isomer, N-oxide, and/or prodrug thereof.

18. A pharmaceutical composition comprising a compound as defined in any one of claims 1 to 17 and one or more excipients.

19. A compound as defined in any one of claims 1 to 17, or a pharmaceutical composition as defined in claim 18, for use as a medicament.

20. A compound as defined in any one of claims 1 to 17, or the pharmaceutical composition as defined in claim 18, for use in the prevention and/or treatment of an inflammatory bowel disease, preferably wherein the inflammatory bowel disease is ulcerative colitis and/or Crohn's disease.

21. A method for the prevention and/or treatment of a disease or condition comprising administering to a subject a compound as defined in any one of claims 1 to 17, wherein the disease or condition is susceptible to PDE10A inhibition.

22. The method as claimed in claim 21, wherein the disease or condition is an inflammatory bowel disease, preferably wherein the inflammatory bowel disease is ulcerative colitis and/or Crohn's disease.

23. Use of a compound as defined in any one of claims 1 to 17 for the manufacture of a medicament.

24. The use as claimed in claim 23, wherein the medicament is for the prevention and/or treatment of an inflammatory bowel disease, preferably wherein the inflammatory bowel disease is ulcerative colitis and/or Crohn's disease.
